(19) United States
(12) Patent Application Publication
(10) Pub. No.: US 2025/0360195 A1
(43) Pub. Date: Nov. 27, 2025

(54) RESPIRATORY SYNCYTIAL VIRUS RNA VACCINATION

(71) Applicant: SANOFI PASTEUR INC., Swiftwater, PA (US)

(72) Inventors: Linong ZHANG, Cambridge, MA (US); Emilie DANVE-CHERY, Lyon Cedex (FR); William Scott GALLICHAN, Toronto, ON (CA); Diana Leticia CORONEL MARTINEZ, Ciudad de Mexico (MX); Olubukola TEMITOPE IDOKO, Lyon Cedex (FR)

(21) Appl. No.: 19/196,288

(22) Filed: May 1, 2025

Related U.S. Application Data
(63) Continuation of application No. PCT/EP2023/080733, filed on Nov. 3, 2023.
(60) Provisional application No. 63/422,621, filed on Nov. 4, 2022.
(60) Provisional application No. 63/523,543, filed on Jun. 27, 2023.

(51) Int. Cl. A61K 39/12; A61K 39/00; A61P 31/14; A61P 37/04; C07K 14/005

(52) U.S. Cl. CPC A61K 39/12; A61P 31/14; A61P 37/04; C07K 14/005; A61K 2039/53; A61K 2039/545; A61K 2039/55; A61K 2039/575; A61K 2039/6018; C12N 2760/18522; C12N 2760/18534

(57) ABSTRACT

The present disclosure provides methods for eliciting an immune response against respiratory syncytial virus (RSV) in a subject. The present disclosure also provides methods for preventing an RSV infection or reducing one or more symptoms of an RSV infection in a subject.

### RELATED APPLICATIONS

This application is a continuation of International Patent Application No. PCT/EP2023/080733, filed Nov. 3, 2023, which claims priority to U.S. Provisional Patent Application Serial No. 63/422,621, filed Nov. 4, 2022, and 63/523,543, filed Jun. 27, 2023, the entire disclosures of which are hereby incorporated by reference in their entireties.

### SEQUENCE LISTING

The instant application contains a Sequence Listing which has been submitted electronically in XML format and is hereby incorporated by reference in its entirety. Said XML file, created on Apr. 30, 2025, is named 763351_SA9-340PCCON_ST26.xml, and is 39,842 bytes in size.

### BACKGROUND OF THE DISCLOSURE

Respiratory syncytial virus (RSV) is a leading cause of severe respiratory disease in infants and a major cause of respiratory illness in the elderly. RSV remains an unmet vaccine need despite decades of research. Recent clinical programs using an RSV F antigen in its post-fusion conformation failed to elicit sufficient efficacy in adults. See, Faloon et al. (2017) JID 216: 1362-1370. However, RSV F antigens stabilized in the pre-fusion conformation elicited much higher neutralizing response superior to that of the post-fusion antigens and thereby potentially confer high level protection efficacy against RSV disease in older adults.

RNA-based vaccines (e.g., mRNA vaccines) have recently emerged as an effective vaccine type against severe acute respiratory syndrome coronavirus 2 (SARS-CoV-2). Coronavirus disease 2019 (COVID-19) mRNA vaccines have exhibited rapid, safe, and cost-effective production processes. Often combined with a delivery vehicle, such as a lipid nanoparticle (LNP), COVID-19 mRNA vaccines can achieve high efficacy. With the dearth of effective RSV vaccines available, there exists a need for RNA-based RSV vaccines that elicit strong immune responses against the RSV pre-fusion F protein for potent neutralization of an RSV infection.

### BRIEF SUMMARY OF THE DISCLOSURE

In certain aspects, a method of eliciting an immune response against respiratory syncytial virus (RSV) in a subject is provided, comprising administering to the subject a prophylactically effective amount of an RSV vaccine comprising a messenger RNA (mRNA) comprising an open reading frame (ORF) encoding an RSV F protein antigen, wherein the RSV F protein antigen comprises an amino acid sequence with at least 98% identity to SEQ ID NO: 3 or consists of an amino acid sequence of SEQ ID NO: 3.

In certain exemplary embodiments, the RSV F protein antigen is a pre-fusion protein.

In certain exemplary embodiments, the RSV vaccine is administered intramuscularly, intranasally, intravenously, subcutaneously, or intradermally. In certain exemplary embodiments, the RSV vaccine is administered intramuscularly. In certain exemplary embodiments, the RSV vaccine is administered in a deltoid muscle of an upper arm of the subject.

In certain exemplary embodiments, the subject is 18 to 50 years of age. In certain exemplary embodiments, the subject is at least 60 years of age.

In certain exemplary embodiments, the RSV vaccine does not comprise an adjuvant.

In certain exemplary embodiments, the mRNA is formulated in a lipid nanoparticle (LNP). In certain exemplary embodiments, the LNP comprises at least one cationic lipid. In certain exemplary embodiments, the at least one cationic lipid is biodegradable or is non-biodegradable. In certain exemplary embodiments, the at least one cationic lipid is cleavable or is non-cleavable. In certain exemplary embodiments, the at least one cationic lipid is selected from the group consisting of OF-02, cKK-E10, GL-HEPES-E3-E10-DS-3-E18-1, GL-HEPES-E3-E12-DS-4-E10, GL-HEPES-E3-E12-DS-3-E14, cKK-E10, or GL-HEPES-E3-E12-DS-4-E10, and IM-001.

In certain exemplary embodiments, the subject is administered an initial dose of the RSV vaccine and one or more booster doses of the RSV vaccine. In certain exemplary embodiments, each of the one or more booster doses is administered to the subject at least 11 months after a previous dose, at least 12 months after a previous dose, about 12 months after a previous dose, or about 10 months to about 14 months after a previous dose.

In certain exemplary embodiments, the subject is administered an initial dose of the RSV vaccine and a booster dose of the RSV vaccine.

In certain exemplary embodiments, the booster dose is administered to the subject at least 11 months after the initial dose, at least 12 months after the initial dose, about 12 months after the initial dose, or about 10 months to about 14 months after the initial dose.

In certain exemplary embodiments, the RSV vaccine is administered at a dose of about 5 micrograms to about 120 micrograms. In certain exemplary embodiments, the RSV vaccine is administered at a dose of about 5 micrograms to about 15 micrograms. In certain exemplary embodiments, the RSV vaccine is administered at a dose of about 10 micrograms.

In certain exemplary embodiments, the RSV vaccine is administered at a dose of about 20 micrograms to about 40 micrograms. In certain exemplary embodiments, the RSV vaccine is administered at a dose of about 30 micrograms.

In certain exemplary embodiments, the RSV vaccine is administered at a dose of about 65 micrograms to about 95 micrograms. In certain exemplary embodiments, the RSV vaccine is administered at a dose of about 75 micrograms.

In certain exemplary embodiments, the RSV vaccine is administered at a dose of about 100 micrograms to about 120 micrograms. In certain exemplary embodiments, the RSV vaccine is administered at a dose of about 110 micrograms.

In other aspects, a method of eliciting an immune response against respiratory syncytial virus (RSV) in a subject is provided, comprising administering to the subject a prophylactically effective amount of an RSV vaccine comprising a messenger RNA (mRNA), wherein the mRNA comprises a nucleic acid sequence with at least 98% identity to SEQ ID NO: 14 or consists of a nucleic acid sequence of SEQ ID NO: 14.

In certain exemplary embodiments, the RSV vaccine is administered intramuscularly, intranasally, intravenously, subcutaneously, or intradermally. In certain exemplary embodiments, the RSV vaccine is administered intramuscularly. In certain exemplary embodiments, the RSV vaccine is administered in a deltoid muscle of an upper arm of the subject.

In certain exemplary embodiments, the subject is 18 to 50 years of age. In certain exemplary embodiments, the subject at least 60 years of age.

In certain exemplary embodiments, the RSV vaccine does not comprise an adjuvant.

In certain exemplary embodiments, the mRNA is formulated in a lipid nanoparticle (LNP). In certain exemplary embodiments, the LNP comprises at least one cationic lipid. In certain exemplary embodiments, the at least one cationic lipid is biodegradable or is non-biodegradable. In certain exemplary embodiments, the at least one cationic lipid is cleavable or is non-cleavable. In certain exemplary embodiments, the at least one cationic lipid is selected from the group consisting of OF-02, cKK-E10, GL-HEPES-E3-E10-DS-3-E18-1, GL-HEPES-E3-E12-DS-4-E10, GL-HEPES-E3-E12-DS-3-E14, cKK-E10, GL-HEPES-E3-E12-DS-4-E10, and IM-001.

In certain exemplary embodiments, the subject is administered an initial dose of the RSV vaccine and one or more booster doses of the RSV vaccine. In certain exemplary embodiments, each of the one or more booster doses is administered to the subject at least 11 months after a previous dose, at least 12 months after a previous dose, about 12 months after a previous dose, or about 10 months to about 14 months after a previous dose.

In certain exemplary embodiments, the subject is administered an initial dose of the RSV vaccine and a booster dose of the RSV vaccine.

In certain exemplary embodiments, the booster dose is administered to the subject at least 11 months after the initial dose, at least 12 months after the initial dose, about 12 months after the initial dose, or about 10 months to about 14 months after the initial dose.

In certain exemplary embodiments, the RSV vaccine is administered at a dose of about 5 micrograms to about 120 micrograms. In certain exemplary embodiments, the RSV vaccine is administered at a dose of about 5 micrograms to about 15 micrograms. In certain exemplary embodiments, the RSV vaccine is administered at a dose of about 10 micrograms.

In certain exemplary embodiments, the RSV vaccine is administered at a dose of about 20 micrograms to about 40 micrograms. In certain exemplary embodiments, the RSV vaccine is administered at a dose of about 30 micrograms.

In certain exemplary embodiments, the RSV vaccine is administered at a dose of about 65 micrograms to about 95 micrograms. In certain exemplary embodiments, the RSV vaccine is administered at a dose of about 75 micrograms.

In other aspects, a method of preventing a respiratory syncytial virus (RSV) infection or reducing one or more symptoms of an RSV infection in a subject is provided, comprising administering to the subject a prophylactically effective amount of an RSV vaccine comprising a messenger RNA (mRNA) comprising an open reading frame (ORF) encoding an RSV F protein antigen, wherein the RSV F protein antigen comprises an amino acid sequence with at least 98% identity to SEQ ID NO: 3 or consists of an amino acid sequence of SEQ ID NO: 3.

In certain exemplary embodiments, the RSV F protein antigen is a pre-fusion protein.

In certain exemplary embodiments, the vaccine is administered intramuscularly, intranasally, intravenously, subcutaneously, or intradermally. In certain exemplary embodiments, the RSV vaccine is administered intramuscularly. In certain exemplary embodiments, the RSV vaccine is administered in a deltoid muscle of an upper arm of the subject.

In certain exemplary embodiments, the subject is 18 to 50 years of age. In certain exemplary embodiments, the subject is at least 60 years of age.

In certain exemplary embodiments, the RSV vaccine does not comprise an adjuvant.

In certain exemplary embodiments, the mRNA is formulated in a lipid nanoparticle (LNP). In certain exemplary embodiments, the LNP comprises at least one cationic lipid. In certain exemplary embodiments, the at least one cationic lipid is biodegradable or is not biodegradable. In certain exemplary embodiments, the at least one cationic lipid is cleavable or is not cleavable. In certain exemplary embodiments, the at least one cationic lipid is selected from the group consisting of OF-02, cKK-E10, GL-HEPES-E3-E10-DS-3-E18-1, GL-HEPES-E3-E12-DS-4-E10, GL-HEPES-E3-E12-DS-3-E14, cKK-E10, GL-HEPES-E3-E12-DS-4-E10, and IM-001.

In certain exemplary embodiments, the subject is administered an initial dose of the RSV vaccine and one or more booster doses of the RSV vaccine. In certain exemplary embodiments, each of the one or more booster doses is administered to the subject at least 11 months after a previous dose, at least 12 months after a previous dose, about 12 months after a previous dose, or about 10 months to about 14 months after a previous dose.

In certain exemplary embodiments, the subject is administered an initial dose of the RSV vaccine and a booster dose of the RSV vaccine. In certain exemplary embodiments, the booster dose is administered to the subject at least 11 months after the initial dose, at least 12 months after the initial dose, about 12 months after the initial dose, or about 10 months to about 14 months after the initial dose.

In certain exemplary embodiments, the RSV vaccine is administered at a dose of about 5 micrograms to about 120 micrograms. In certain exemplary embodiments, the RSV vaccine is administered at a dose of about 5 micrograms to about 15 micrograms. In certain exemplary embodiments, the RSV vaccine is administered at a dose of about 10 micrograms.

In certain exemplary embodiments, vaccine is administered at a dose of about 20 micrograms to about 40 micrograms. In certain exemplary embodiments, the RSV vaccine is administered at a dose of about 30 micrograms.

In certain exemplary embodiments, the RSV vaccine is administered at a dose of about 65 micrograms to about 95 micrograms. In certain exemplary embodiments, the RSV vaccine is administered at a dose of about 75 micrograms.

In certain exemplary embodiments, the RSV vaccine is administered at a dose of about 100 micrograms to about 120 micrograms. In certain exemplary embodiments, the RSV vaccine is administered at a dose of about 110 micrograms.

In certain exemplary embodiments, the one or more symptoms of an RSV infection are selected from the group consisting of acute respiratory disease (ARD), medically attended acute respiratory disease (MAARD), severe ARD, non-medically attended lower respiratory tract disease (LRTD), medically attended LRTD, congestion, runny nose, cough, fever, sore throat, headache, pneumonia, bronchiolitis, bronchopneumonia, and tracheobronchitis.

In other aspects, a method of preventing a respiratory syncytial virus (RSV) infection or reducing one or more symptoms of an RSV infection in a subject is provided, comprising administering to the subject a prophylactically effective amount of an RSV vaccine comprising a messenger RNA (mRNA), wherein the mRNA comprises a nucleic acid sequence with at least 98% identity to SEQ ID NO: 14 or consists of a nucleic acid sequence of SEQ ID NO: 14.

In certain exemplary embodiments, the vaccine is administered intramuscularly, intranasally, intravenously, subcutaneously, or intradermally. In certain exemplary embodiments, the RSV vaccine is administered intramuscularly. In certain exemplary embodiments, the RSV vaccine is administered in a deltoid muscle of an upper arm of the subject.

In certain exemplary embodiments, the subject is 18 to 50 years of age. In certain exemplary embodiments, the subject is at least 60 years of age.

In certain exemplary embodiments, the RSV vaccine does not comprise an adjuvant.

In certain exemplary embodiments, the mRNA is formulated in a lipid nanoparticle (LNP). In certain exemplary embodiments, the LNP comprises at least one cationic lipid. In certain exemplary embodiments, the at least one cationic lipid is biodegradable or is not biodegradable. In certain exemplary embodiments, the at least one cationic lipid is cleavable or is not cleavable. In certain exemplary embodiments, the at least one cationic lipid is selected from the group consisting of OF-02, cKK-E10, GL-HEPES-E3-E10-DS-3-E18-1, GL-HEPES-E3-E12-DS-4-E10, GL-HEPES-E3-E12-DS-3-E14, cKK-E10, GL-HEPES-E3-E12-DS-4-E10, and IM-001.

In certain exemplary embodiments, the subject is administered an initial dose of the RSV vaccine and one or more booster doses of the RSV vaccine. In certain exemplary embodiments, each of the one or more booster doses is administered to the subject at least 11 months after a previous dose, at least 12 months after a previous dose, about 12 months after a previous dose, or about 10 months to about 14 months after a previous dose.

In certain exemplary embodiments, the subject is administered an initial dose of the RSV vaccine and a booster dose of the RSV vaccine. In certain exemplary embodiments, the booster dose is administered to the subject at least 11 months after the initial dose, at least 12 months after the initial dose, about 12 months after the initial dose, or about 10 months to about 14 months after the initial dose.

In certain exemplary embodiments, the RSV vaccine is administered at a dose of about 5 micrograms to about 120 micrograms. In certain exemplary embodiments, the RSV vaccine is administered at a dose of about 5 micrograms to about 15 micrograms. In certain exemplary embodiments, the RSV vaccine is administered at a dose of about 10 micrograms.

In certain exemplary embodiments, vaccine is administered at a dose of about 20 micrograms to about 40 micrograms. In certain exemplary embodiments, the RSV vaccine is administered at a dose of about 30 micrograms.

In certain exemplary embodiments, the RSV vaccine is administered at a dose of about 65 micrograms to about 95 micrograms. In certain exemplary embodiments, the RSV vaccine is administered at a dose of about 75 micrograms.

In certain exemplary embodiments, the one or more symptoms of an RSV infection are selected from the group consisting of acute respiratory disease (ARD), medically attended acute respiratory disease (MAARD), severe ARD, non-medically attended lower respiratory tract disease (LRTD), medically attended LRTD, congestion, runny nose, cough, fever, sore throat, headache, pneumonia, bronchiolitis, bronchopneumonia, and tracheobronchitis.

In other aspects, a method of eliciting an immune response against respiratory syncytial virus (RSV) in a subject is provided, comprising selecting a subject that is 18 to 50 years of age or is at least 60 years of age, and administering to the subject a prophylactically effective amount of an RSV vaccine comprising a messenger RNA (mRNA) comprising an open reading frame (ORF) encoding an RSV F protein antigen, wherein the RSV F protein antigen comprises an amino acid sequence with at least 98% identity to SEQ ID NO: 3 or consists of an amino acid sequence of SEQ ID NO: 3.

In other aspects, a method of eliciting an immune response against respiratory syncytial virus (RSV) in a subject is provided, comprising selecting a subject that is 18 to 50 years of age or is at least 60 years of age, and administering to the subject a prophylactically effective amount of an RSV vaccine comprising a messenger RNA (mRNA), wherein the mRNA comprises a nucleic acid sequence with at least 98% identity to SEQ ID NO: 14 or consists of a nucleic acid sequence of SEQ ID NO: 14.

In other aspects, a method of preventing a respiratory syncytial virus (RSV) infection or reducing one or more symptoms of an RSV infection in a subject is provided, comprising selecting a subject that is 18 to 50 years of age or is at least 60 years of age, and administering to the subject a prophylactically effective amount of an RSV vaccine comprising a messenger RNA (mRNA) comprising an open reading frame (ORF) encoding an RSV F protein antigen, wherein the RSV F protein antigen comprises an amino acid sequence with at least 98% identity to SEQ ID NO: 3 or consists of an amino acid sequence of SEQ ID NO: 3.

In other aspects, a method of preventing a respiratory syncytial virus (RSV) infection or reducing one or more symptoms of an RSV infection in a subject is provided, comprising selecting a subject that is 18 to 50 years of age or is at least 60 years of age, and administering to the subject a prophylactically effective amount of an RSV vaccine comprising a messenger RNA (mRNA), wherein the mRNA comprises a nucleic acid sequence with at least 98% identity to SEQ ID NO: 14 or consists of a nucleic acid sequence of SEQ ID NO: 14.

In other aspects, a respiratory syncytial virus (RSV) vaccine for use in eliciting an immune response against RSV in a subject is provided, wherein the RSV vaccine comprises a messenger RNA (mRNA) comprising an open reading frame (ORF) encoding an RSV F protein antigen, wherein the RSV F protein antigen comprises an amino acid sequence with at least 98% identity to SEQ ID NO: 3 or consists of an amino acid sequence of SEQ ID NO: 3, and wherein the RSV F protein antigen is a pre-fusion protein.

In other aspects, a respiratory syncytial virus (RSV) vaccine for use in eliciting an immune response against RSV in a subject is provided, wherein the RSV vaccine comprises a messenger RNA (mRNA) comprising an open reading frame (ORF) encoding an RSV F protein antigen, wherein the RSV F protein antigen comprises an amino acid sequence with at least 98% identity to SEQ ID NO: 3 or consists of an amino acid sequence of SEQ ID NO: 3.

In other aspects, a respiratory syncytial virus (RSV) vaccine for use in eliciting an immune response against RSV in a subject is provided, wherein the RSV vaccine comprises a messenger RNA (mRNA), wherein the mRNA comprises a nucleic acid sequence with at least 98% identity to SEQ ID NO: 14 or consists of a nucleic acid sequence of SEQ ID NO: 14.

In other aspects, a respiratory syncytial virus (RSV) vaccine for use in preventing RSV infection or reducing one or more symptoms of an RSV infection in a subject is provided, wherein the RSV vaccine comprises a messenger RNA (mRNA) comprising an open reading frame (ORF) encoding an RSV F protein antigen, wherein the RSV F protein antigen comprises an amino acid sequence with at least 98% identity to SEQ ID NO: 3 or consists of an amino acid sequence of SEQ ID NO: 3.

In other aspects, a respiratory syncytial virus (RSV) vaccine for use in preventing RSV infection or reducing one or more symptoms of an RSV infection in a subject is provided, wherein the RSV vaccine comprises a messenger RNA (mRNA), wherein the mRNA comprises a nucleic acid sequence with at least 98% identity to SEQ ID NO: 14 or consists of a nucleic acid sequence of SEQ ID NO: 14.

In other aspects, a method of eliciting an immune response against respiratory syncytial virus (RSV) in a subject is provided, the method comprising selecting a subject that is at least 60 years of age, and administering to the subject an RSV vaccine comprising a messenger RNA (mRNA), wherein the mRNA comprises a nucleic acid sequence with at least 98% identity to SEQ ID NO: 14 or consists of a nucleic acid sequence of SEQ ID NO: 14, wherein the mRNA is formulated in a lipid nanoparticle (LNP) comprising GL-HEPES-E3-E12-DS-4-E10, and wherein the RSV vaccine is administered at a dose of about 110 micrograms.

In other aspects, a method of eliciting an immune response against respiratory syncytial virus (RSV) in a subject is provided, the method comprising selecting a subject that is at least 60 years of age, and administering an RSV vaccine comprising a messenger RNA (mRNA), wherein the mRNA comprises a nucleic acid sequence with at least 98% identity to SEQ ID NO: 14 or consists of a nucleic acid sequence of SEQ ID NO: 14, wherein the mRNA is formulated in a lipid nanoparticle (LNP) comprising GL-HEPES-E3-E12-DS-4-E10, and wherein the RSV vaccine is administered at a dose of about 75 micrograms.

In other aspects, a method of eliciting an immune response against respiratory syncytial virus (RSV) in a subject is provided, the method comprising selecting a subject that is at least 60 years of age, and administering to the subject an RSV vaccine comprising a messenger RNA (mRNA), wherein the mRNA comprises a nucleic acid sequence with at least 98% identity to SEQ ID NO: 14 or consists of a nucleic acid sequence of SEQ ID NO: 14, wherein the mRNA is formulated in a lipid nanoparticle (LNP) comprising GL-HEPES-E3-E12-DS-4-E10, and wherein the RSV vaccine is administered at a dose of about 30 micrograms.

In other aspects, a method of eliciting an immune response against respiratory syncytial virus (RSV) in a subject is provided, the method comprising selecting a subject that is at least 60 years of age, and administering to the subject an RSV vaccine comprising a messenger RNA (mRNA), wherein the mRNA comprises a nucleic acid sequence with at least 98% identity to SEQ ID NO: 14 or consists of a nucleic acid sequence of SEQ ID NO: 14, wherein the mRNA is formulated in a lipid nanoparticle (LNP) comprising cKK-E10, and wherein the RSV vaccine is administered at a dose of about 30 micrograms.

In other aspects, a method of eliciting an immune response against respiratory syncytial virus (RSV) in a subject is provided, the method comprising selecting a subject that is at least 60 years of age, and administering to the subject an RSV vaccine comprising a messenger RNA (mRNA), wherein the mRNA comprises a nucleic acid sequence with at least 98% identity to SEQ ID NO: 14 or consists of a nucleic acid sequence of SEQ ID NO: 14, wherein the mRNA is formulated in a lipid nanoparticle (LNP) comprising cKK-E10, and wherein the RSV vaccine is administered at a dose of about 75 micrograms.

In other aspects, a method of eliciting an immune response against respiratory syncytial virus (RSV) in a subject is provided, the method comprising: selecting a subject that is at least 60 years of age; and administering to the subject an RSV vaccine comprising a messenger RNA (mRNA), wherein the mRNA comprises a nucleic acid sequence with at least 98% identity to SEQ ID NO: 14 or consists of a nucleic acid sequence of SEQ ID NO: 14, wherein the mRNA is formulated in a lipid nanoparticle (LNP) comprising cKK-E10, and wherein the RSV vaccine is administered at a dose of about 110 micrograms.

In other aspects, a method of eliciting an immune response against respiratory syncytial virus (RSV) in a subject is provided, the method comprising: selecting a subject that is at least 60 years of age; and administering to the subject an RSV vaccine comprising a messenger RNA (mRNA), wherein the mRNA comprises a nucleic acid sequence with at least 98% identity to SEQ ID NO: 14 or consists of a nucleic acid sequence of SEQ ID NO: 14, wherein the mRNA is formulated in a lipid nanoparticle (LNP) comprising IM-001, and wherein the RSV vaccine is administered at a dose of about 30 micrograms.

In other aspects, a method of eliciting an immune response against respiratory syncytial virus (RSV) in a subject is provided, the method comprising: selecting a subject that is at least 60 years of age; and administering to the subject an RSV vaccine comprising a messenger RNA (mRNA), wherein the mRNA comprises a nucleic acid sequence with at least 98% identity to SEQ ID NO: 14 or consists of a nucleic acid sequence of SEQ ID NO: 14, wherein the mRNA is formulated in a lipid nanoparticle (LNP) comprising IM-001, and wherein the RSV vaccine is administered at a dose of about 75 micrograms.

In other aspects, a method of eliciting an immune response against respiratory syncytial virus (RSV) in a subject is provided, the method comprising: selecting a subject that is at least 60 years of age; and administering to the subject an RSV vaccine comprising a messenger RNA (mRNA), wherein the mRNA comprises a nucleic acid sequence with at least 98% identity to SEQ ID NO: 14 or consists of a nucleic acid sequence of SEQ ID NO: 14, wherein the mRNA is formulated in a lipid nanoparticle (LNP) comprising IM-001, and wherein the RSV vaccine is administered at a dose of about 110 micrograms.

### BRIEF DESCRIPTION OF THE DRAWINGS/FIGURES

The foregoing and other features and advantages of the present disclosure will be more fully understood from the following detailed description of illustrative embodiments taken in conjunction with the accompanying drawings.

FIG. 1 graphically depicts an overview of the study design for the Study A cohort (Sentinel Cohort, ages 18 to 50 years old). AE: adverse event; AESI: adverse event of special interest; BL: blood sample; MAAE: medically attended adverse event; RSV: respiratory syncytial virus; SAE: serious adverse event; SCR: Screening. Note: D01 (V01) blood sampling to be completed prior to vaccination.

FIG. 2 graphically depicts an overview of the study design for the Study B cohort (Main Cohort, 60 years old and older). AE: adverse event; AESI: adverse event of special interest; BL: blood samples for immunogenicity; MAAE: medically attended adverse event; RSV: respiratory syncytial virus; SAE: serious adverse event SCR: Screening; VAC: vaccine; WB: blood sample for CMI; * D04 (V02) does not apply to the Main Cohort; † D01 (V01) blood sampling to be completed prior to vaccination.

FIG. 3 graphically depicts an overview of the study design for the Study C cohort (Booster Cohort, 60 years old and older). AE: adverse event; AESI: adverse event of special interest; BL: blood samples for immunogenicity; MAAE: medically attended adverse event; RSV: respiratory syncytial virus; SAE: serious adverse event; SCR: Screening. Note 1: Approximately 200 participants, 100 participants from the selected formulation group and 100 participants from the placebo group, will be randomized at M12 in a 1:1 ratio to receive a booster vaccination of the selected formulation of RSV mRNA vaccine (i.e., the dose-level and the LNP formulation selected based upon the Main Cohort safety and immunogenicity results) or placebo. Note 2: BL0005, obtained at V07 of Main Cohort, will be used as the pre-vaccination sample in the Booster Cohort.

FIG. 4 depicts a table showing the schedule of activities for the Study A cohort (Sentinel Cohort, participants aged 18 to 50 years). AE=adverse event; AESI=adverse event of special interest; BL=blood sampling for immunogenicity; BS=blood sampling for assessment of safety; CRF=case report form; D or d=day(s); DC=diary card; M=month; MA=memory aid; MAAE=medically attended adverse events; NS=nasal swab; PRN=as needed; SAE=serious adverse event; TC=telephone call; UN=blood sampling for illness visit; V=visit; vac=vaccination. One asterisk (*) symbolizes that non-site visit contacts are to be made by phone at scheduled timepoints in the study. Dagger (†) symbolizes an abbreviated physical examination for all in-person visits after Visit 01. Double dagger (‡) symbolizes that an electrocardiogram (ECG) to be performed at Screening as a baseline and reviewed by investigator for features of previous myocarditis, pericarditis, and/or myopericarditis. Additional ECG will be performed as rapidly as possible (i.e., at an unscheduled visit, if necessary) for any participant who develops symptoms of myocarditis, pericarditis, and/or myopericarditis during conduct of the study. Section mark (§) indicates that temperature is to be measured by oral route (preferred) or axillary route using a standard digital thermometer and recorded in the source document. Two asterisks (**) symbolizes that the safety laboratory assessments will include serum chemistries, hematology, and coagulation times. At Screening and V03 a serum volume sample will be taken for Troponin I level testing as part of the safety laboratory assessments; part of the samples taken at V04, V05, V06 and V07 will be stored for potential future testing of Troponin I in the event that a participant develops symptoms of myocarditis, pericarditis, and/or myopericarditis (Screening blood sample will be used as baseline). In case of abnormal safety laboratory results, unscheduled visits may occur based on investigator's judgment. The blood volumes of safety laboratories may be adjusted based on local regulations. Two daggers (††) symbolizes that a nasal swab specimen for the detection of RSV and respiratory pathogens (including COVID-19) will be collected from participants during illness visits including medically attended visits during the study. If a participant visits any other non-study doctor/hospital for a serious adverse event at any time in the study, a nasal swab sample will be obtained at the study site once the subject is discharged, if deemed appropriate by the investigator. All nasal swab specimens will be collected in the recommended viral transport media tube and will be stored at −60° C. to −80° C. until ready to ship. The requirement for an at home or on-site illness visit will be evaluated first by video call (preferred) or regular phone call if video call is not possible, to enable remote evaluation of severity and remote management of mild (Grade 1) illness, as deemed appropriate by the investigator. Two double daggers (‡‡) symbolizes any unsolicited systemic adverse events occurring within the 30 minutes from vaccine administration will be recorded as immediate unsolicited systemic adverse events in the case report form. Two section marks (§ §) symbolizes the participant will record information in a diary card about solicited reactions, unsolicited adverse events (AEs), and medically attended AEs from day 0 (D0) to day 28 (D28) after vaccine administration, and AESIs and SAEs throughout the study. Three asterisks (***) symbolize only medications that may have an impact on the immune response or that may have an impact on both the safety and immune response will be collected. Three daggers (†††) symbolize that in the case of participant discontinuation at a visit, the entire visit will be completed. Three double daggers (‡‡‡) or an “X” indicates that the nasopharyngeal swab number will be unique to each site. A nasopharyngeal swab for central laboratory testing will be collected. A sample for local laboratory testing (clinical care) may be collected in addition to the UN swab for central laboratory testing. The swab will not be collected during an Illness Visit if the date of collection is more than 14 days after resolution of symptoms.

FIG. 5 depicts a table showing the schedule of activities for the Study B cohort (Main Cohort, participants aged 60 years and older). AE=adverse event; AESI=adverse event of special interest; BL=blood sampling for immunogenicity; BS=blood sampling for assessment of safety; CMI=cell-mediated immunity; CRF=case report form; D or d=Days; DC=diary card; M=month(s); MA=memory aid; MAAE=medically attended adverse event; NS=nasal swab; PRN=as needed; SAE=serious adverse event; TC=telephone call; UN=blood sample for illness visit; V=visit; vac: vaccination; WB=blood sample for TruCulture. One asterisk (*) symbolizes that the day 04 visit (V02) does not apply to the Main Cohort. A dagger (†) indicates that that the non-site visit contacts are to be made by phone at scheduled timepoints in the study. A double dagger (‡) indicates an abbreviated physical examination for all in-person visits after Visit 01. A section mark (§) indicates an electrocardiogram is performed at Screening as a baseline, and reviewed by investigator for features of previous myocarditis, pericarditis, and/or myopericarditis. Additional ECG will be performed as rapidly as possible (i.e., at an unscheduled visit, if necessary) for any participant who develops symptoms of myocarditis, pericarditis, and/or myopericarditis during conduct of the study. A double asterisk (**) symbolizes temperature to be measured by oral route (preferred) or axillary route using a standard digital thermometer and recorded in the source document. Two daggers (††) indicate that samples collected from a subset of 140 participants for CMI assays are assessed by TruCulture. Two double daggers (‡‡) indicates safety laboratory assessments will include serum chemistries, hematology, and coagulation times. At Screening and at V03, a serum volume sample will be taken for Troponin I level as part of the safety laboratory assessments; part of the samples taken at V01, V04, V05, V06 and V07 will be stored for potential future testing of Troponin I in the event that a participant develops symptoms of myocarditis, pericarditis, and/or myopericarditis (Screening blood sample will be used as baseline). In case of abnormal safety laboratory results, unscheduled visits may occur based on Investigator's judgment. Two section marks (§ §) indicates that the nasal swab specimen for the detection of RSV and respiratory pathogens (including COVID-19) will be collected from participants during illness visits including medically attended visits during the study. If a participant visits any other non-study doctor/hospital for an SAE at any time in the study, a nasal swab sample will be obtained at the study site once the subject is discharged, if deemed appropriate by the investigator. All nasal swab specimens will be collected in the recommended viral transport media tube and will be stored at −60° C. to −80° C. until ready to ship. The requirement for an at home or on-site illness visit will be evaluated first by video call (preferred) or regular phone call if video call is not possible, to enable remote evaluation of severity and remote management of mild (Grade 1) illness, as deemed appropriate by the study investigator. Three asterisks (***) indicates any unsolicited systemic AEs occurring within the 30 minutes from vaccine administration will be recorded as immediate unsolicited systemic AEs in the case report form. Three daggers (†††) indicates the participant will record information in a diary card about solicited reactions and unsolicited AEs and MAAES from day 0-day 28 after vaccine administration and AESIs and SAEs throughout the study. Three double daggers (‡‡‡) indicates that only medications that may have an impact on the immune response or that may have an impact on both the safety and immune response will be collected. Three section marks (§ § §) indicates that in case of participant discontinuation at a visit, the entire visit will be completed. Four asterisks (****) or an “X” indicates that the nasopharyngeal swab number will be unique to each site. A nasopharyngeal swab for central laboratory testing will be collected. A sample for local laboratory testing (clinical care) may be collected in addition to the UN swab for central laboratory testing. The swab will not be collected during an Illness Visit if the date of collection is more than 14 days after resolution of symptoms. Four double daggers (††††) indicates participants who will not continue to Booster Cohort.

FIG. 6 depicts a table showing the schedule of activities for the Study C cohort (Booster Cohort, participants aged 60 years and older). AE=adverse events; AESI=adverse event of special interest; BL=blood sampling for immunogenicity; BS=blood sampling for assessment of safety; CRF=case report form; D or d=day; DC=diary card; M=month; MA=memory aid; MAAE=medically attended adverse event; NS=nasal swab; PRN=as needed; SAE=serious adverse event; TC=telephone call; UN=blood sample for illness visit; V=visit; vac=vaccination; Vac2=booster vaccination. An asterisk (*) indicates non-site visit contacts are to be made by phone at scheduled timepoints in the study. A dagger (†) indicates an abbreviated physical examination for all in-person visits after Visit 08. Temperature is to be measured by oral route (preferred) or axillary route using a standard digital thermometer and recorded in the source document. A double dagger (‡) symbolizes that an electrocardiogram (ECG) is performed at Screening as a baseline, and reviewed by investigator for features of previous myocarditis, pericarditis, and/or myopericarditis. Additional ECG will be performed as rapidly as possible (i.e., at an unscheduled visit, if necessary) for any participant who develops symptoms of myocarditis, pericarditis, and/or myopericarditis during conduct of the study. A section mark (§) indicates that temperature is to be measured by oral route (preferred) or axillary route using a standard digital thermometer and recorded in the source document. Two asterisks (**) annotates blood sample 5, called BL0005, obtained at V07, to be used as the pre-vaccination sample in the Booster Cohort. A double dagger (††) indicates that safety laboratory assessments will include serum chemistries, hematology, and coagulation times. At Screening and at V09, a serum volume sample will be taken for troponin I level testing as part of the safety laboratory assessments; part of the samples taken at V10, V11, V12 and V13 will be stored for potential future testing of Troponin I in the event that a participant develops symptoms of myocarditis, pericarditis, and/or myopericarditis (screening blood sample will be used as baseline). In case of abnormal safety laboratory results, unscheduled visits may occur based on Investigator's judgment. Two double daggers (‡‡) indicates that the nasal swab specimen for the detection of RSV and respiratory pathogens (including COVID-19) will be collected from participants during illness visits including medically attended visits during the study. If a participant visits any other non-study doctor/hospital for an SAE at any time in the study, a nasal swab sample will be obtained at the study site once the subject is discharged, if deemed appropriate by the study investigator. If the participant cannot attend in-site illness visit and/or not receive a home visit, self-collection of the sample should be performed. All nasal swab specimens will be collected in the recommended viral transport media tube and will be stored at −60° C. to −80° C. until ready to ship. The requirement for an at home or on-site illness visit will be evaluated first by video call (preferred) or regular phone call if video call is not possible, to enable remote evaluation of severity and remote management of mild (Grade 1) illness, as deemed appropriate by the study investigator. Two section marks (§ §) indicates that it is to be administered 12 months after the first injection. Three asterisks (***) symbolizes any unsolicited systemic AEs occurring within the 30 minutes from vaccine administration will be recorded as immediate unsolicited systemic AEs in the case report form. Three daggers (†††) indicates that the participant will record information in a diary card about solicited reactions, unsolicited AEs, and MAAEs from day 0-day 28 (D0 to D28) after vaccine administration and AESIs and SAEs throughout the study. Three double daggers (‡‡‡) indicates that the only medications that may have an impact on the immune response or that may have an impact on both the safety and immune response will be collected. Three section marks (§ § §) or “X” indicates that the nasopharyngeal swab number will be unique to each site. A nasopharyngeal swab for central laboratory testing will be collected. A sample for local laboratory testing (clinical care) may be collected in addition to the UN swab for central laboratory testing. The swab will not be collected during an Illness Visit if the date of collection is more than 14 days after resolution of symptoms.

FIG. 7 is a table depicting demographic characteristics of the Main Cohort.

FIG. 8 graphically depicts a summary of geometric mean titers (GMTs) of RSV-A neutralizing antibody (NAb) titers after primary vaccination and neutralizing antibody geometric mean titer ratios (GMTRs) at D29 and D01. X-axis: 1=cKK-E10, 10 μg, n=93; 2=cKK-E10, 30 μg, n=95; 3=cKK-E10, 75 μg, n=97; 4=GL-HEPES-E3-E12-DS-4-E10, 10 μg, n=99; 5=GL-HEPES-E3-E12-DS-4-E10, 30 μg, n=96; and 6=GL-HEPES-E3-E12-DS-4-E10, 75 μg, n=92.

FIG. 9 graphically depicts participants with ≥4-fold and <4-fold rise for RSV-A neutralizing antibody titers after primary vaccination for the full Main Cohort (age 60 years and above).

FIG. 10 graphically depicts a partial Main Cohort (age 60 years and above) summarizing geometric means of IgG antibody titers after primary vaccination and IgG antibody geometric mean titer ratios at D29 and D01. X-axis: 1=cKK-E10, 10 μg, n=38; 2=cKK-E10, 30 μg, n=35; 3=cKK-E10, 75 μg, n=44; 4=GL-HEPES-E3-E12-DS-4-E10, 10 μg, n=45; 5=GL-HEPES-E3-E12-DS-4-E10, 30 μg, n=41; and 6=GL-HEPES-E3-E12-DS-4-E10, 75 μg, n=38.

FIG. 11 graphically depicts a summary of geometric mean titers (GMTs) of RSV-A neutralizing antibody (NAb) titers after primary vaccination and neutralizing antibody geometric mean titer ratios (GMTRs) at D29 and D01 for the Sentinel Cohorts (age 18-50 years). X-axis: 1=cKK-E10, 10 μg, n=9; 2=cKK-E10, 30 μg, n=8; 3=cKK-E10, 75 μg, n=10; 4=GL-HEPES-E3-E12-DS-4-E10, 10 μg, n=7; 5=GL-HEPES-E3-E12-DS-4-E10, 30 μg, n=10; and 6=GL-HEPES-E3-E12-DS-4-E10, 75 μg, n=10.

FIG. 12 graphically depicts participants with ≥4-fold and <4-fold rise for RSV-A neutralizing antibody titers after primary vaccination for the Sentinel Cohorts (age 18-50 years).

FIG. 13A-FIG. 13B graphically summarizes fold-rise for RSV-A neutralizing antibody titers after primary vaccination for the Sentinel Cohorts. (FIG. 13A) cKK-E10. (FIG. 13B) GL-HEPES-E3-E12-DS-4-E10.

FIG. 14 graphically summarizes solicited reactions within 7 days after primary vaccination by percent. X-Axis: A=cKK-E10; B=GL-HEPES-E3-E12-DS-4-E10; C=placebo.

FIG. 15 graphically summarizes solicited injection site reactions within 7 days after primary vaccination by percent. X-Axis: A=cKK-E10; B=GL-HEPES-E3-E12-DS-4-E10; C=placebo.

FIG. 16 graphically summarizes solicited systemic reactions within 7 days after primary vaccination by percent. X-Axis: A=cKK-E10; B=GL-HEPES-E3-E12-DS-4-E10; C=placebo.

FIG. 17 is a table summarizing the safety overview after primary vaccination (Main Cohort).

FIG. 18 is a table summarizing solicited reactions within 7 days after primary vaccination (Main Cohort).

FIG. 19 is a table summarizing unsolicited adverse events. As no dose responses were observed, pooled data are presented. AESI downgraded as non-AESI by investigator after cut-off date of biostatistical output (diagnosis changed from myocarditis to Asymptomatic myocardial injury). *No meaningful imbalance, only numerical observed unbalanced driven by musculoskeletal and connective tissue disorders (such as arthralgia, myalgia, muscle spasms), and gastrointestinal disorders (such as abdominal pain, diarrhea, nausea). ^{δ}SAEs were equally distributed across mRNA groups (two in LNP cKK-E10 and three in LNP GL-HEPES-E3-E12-DS-4-E10). Only one SAE (asymptomatic myocardial injury) was assessed as related to IMP (LNP cKK-E10 low dose). Events were: hypotension with dehydration and syncope in LNP cKK-E10 (low dose or high dose); Asymptomatic Myocardial Injury in LNP cKK-E10 (low dose); Constipation in LNP GL-HEPES-E3-E12-DS-4-E10 (low dose); Hydronephrosis with Pyelonephritis and RSV infection in LNP GL-HEPES-E3-E12-DS-4-E10 (medium dose); Hypertensive crisis in LNP GL-HEPES-E3-E12-DS-4-E10 (high dose).

FIG. 20 is a table summarizing unsolicited adverse events compared to placebo due to musculoskeletal disorders, connective tissue disorders, and gastrointestinal disorders.

### DETAILED DESCRIPTION OF THE DISCLOSURE

The present disclosure is directed to, inter alia, RNA (e.g., mRNA) vaccine compositions encoding an RSV F protein and methods of vaccination with the same. Furthermore, the disclosure relates to vaccine compositions comprising mRNA encoding an RSV pre-fusion F protein formulated in a lipid nanoparticle (LNP) and methods of vaccination with the same.

### I. Definitions

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, protein and nucleic acid chemistry, and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, may provide one of skill with a general dictionary of many of the terms used in this disclosure.

Units, prefixes, and symbols are denoted in their International System of Units (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

The terms “approximately” or “about” are used herein to mean roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

As used herein, the term “messenger RNA” or “mRNA” refers to a polynucleotide that encodes at least one polypeptide. mRNA, as used herein, encompasses both modified and unmodified RNA. mRNA may contain one or more coding and non-coding regions. A coding region is alternatively referred to as an open reading frame (ORF). Non-coding regions in mRNA include the 5′ cap, 5′ untranslated region (UTR), 3′ UTR, and a poly(A) tail. mRNA can be purified from natural sources or produced using recombinant expression systems (e.g., in vitro transcription). In various embodiments, mRNA can be purified or chemically synthesized.

As used herein, the term “F protein” or “RSV F protein” refers to the protein of RSV responsible for driving fusion of the viral envelope with host cell membrane during viral entry.

As used herein, the term “RSV F polypeptide” or “F polypeptide” refers to a polypeptide comprising at least one epitope of F protein.

As used herein, the term “post-fusion” with respect to RSV F refers to a stable conformation of RSV F that occurs after merging of the virus and cell membranes.

As used herein, the term “pre-fusion” with respect to RSV F refers to a conformation of RSV F that is adopted before virus-cell interaction.

As used herein, the term “protomer” refers to a structural unit of an oligomeric protein. In the case of RSV F, an individual unit of the RSV F trimer is a protomer.

As used herein, the term “N-glycan” refers to a saccharide chain attached to a protein at the amide nitrogen of an N (asparagine) residue of the protein. As such, an N-glycan is formed by the process of N-glycosylation. This glycan may be a polysaccharide.

As used herein, the term “glycosylation” refers to the addition of a saccharide unit to a protein.

As used herein, the term “immune response” refers to a response of a cell of the immune system, such as a B cell, T cell, dendritic cell, macrophage, or polymorphonucleocyte to a stimulus such as an antigen or vaccine. An immune response can include any cell of the body involved in a host defense response, including, for example, an epithelial cell that secretes an interferon or a cytokine. An immune response includes, but is not limited to, an innate and/or adaptive immune response.

As used herein, an “antibody response” is an immune response in which antibodies are produced.

As used herein, an “antigen” refers to an agent that elicits an immune response, and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism. Alternatively, or additionally, in some embodiments, an antigen elicits a cellular response (e.g., involving T-cells whose receptors specifically interact with the antigen) in an organism. A particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity. Antigens include the RSV polypeptides encoded by the mRNA as described herein.

As used herein, an “adjuvant” refers to a substance or vehicle that enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes, killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as toll-like receptor (TLR) agonists and costimulatory molecules.

As used herein, a “subject” refers to any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically engineered animal, and/or a clone. In certain embodiments, the subject is an adult, an adolescent, or an infant. In some embodiments, the terms “individual” or “patient” are used and are intended to be interchangeable with “subject.” In certain exemplary embodiments, the subject is a preterm newborn infant (e.g., gestational age less than 37 weeks), a newborn (e.g., 0-27 days of age), an infant or toddler (e.g., 28 days to 23 months of age), a child (e.g., 2 to 11 years of age), an adolescent (e.g., 12 to 17 years of age), an adult (e.g., 18 to 50 years of age or 18 to 64 years of age), or an elderly person (e.g., 65 years of age or older). In exemplary embodiments, the subject is an older adult (e.g., an adult aged 60 years of age or older).

As used herein, the term “vaccination” or “vaccinate” refers to the administration of a composition intended to generate an immune response, for example, to a disease-causing agent. Vaccination can be administered before, during, and/or after exposure to a disease-causing agent, and/or to the development of one or more symptoms, and in some embodiments, before, during, and/or shortly after exposure to the disease-causing agent. In some embodiments, vaccination includes multiple administrations, appropriately spaced in time, of a vaccinating composition, e.g., administration approximately 12 months after a previous dose.

The disclosure describes nucleic acid sequences (e.g., DNA and RNA sequences) and amino acid sequences having a certain degree of identity to a given nucleic acid sequence or amino acid sequence, respectively (a reference sequence).

The terms “% identical,” “% identity,” or similar terms are intended to refer, in particular, to the percentage of nucleotides or amino acids which are identical in an optimal alignment between the sequences to be compared. Said percentage is purely statistical, and the differences between the two sequences may be, but are not necessarily, randomly distributed over the entire length of the sequences to be compared. “Sequence identity” between two nucleic acid sequences indicates the percentage of nucleotides that are identical between the sequences. “Sequence identity” between two amino acid sequences indicates the percentage of amino acids that are identical between the sequences. Comparisons of two sequences are usually carried out by comparing said sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N, and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90%, or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments, in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. In some embodiments, a nucleic acid sequence or amino acid sequence having a particular degree of identity to a given nucleic acid sequence or amino acid sequence is functionally equivalent to said given sequence.

As used herein, the term “kit” refers to a packaged set of related components, such as one or more compounds or compositions and one or more related materials such as solvents, solutions, buffers, instructions, or desiccants.

### II. RSV mRNA Vaccines

Respiratory syncytial virus (RSV) is a negative-sense, single-stranded RNA virus belonging to the Pneumoviridae family. RSV can cause infection of the respiratory tract. RSV is an enveloped virus. The surface of the RSV virion contains 3 proteins: the attachment glycoprotein (G), the fusion protein (F), and the small hydrophobic (SH) protein.

The RSV F protein is responsible for fusion of viral and host cell membranes and takes on at least three conformations (pre-fusion, intermediate, and post-fusion conformations). In the pre-fusion conformation (pre-fusion, Pre-F), the F protein exists in a trimeric form with the major antigenic site Ø exposed. Site Ø serves as a primary target of neutralizing antibodies produced by RSV-infected subjects (see, Coultas et al., Thorax. 74: 986-993. 2019; McLellan et al., Science. 340(6136): 1113-7. 2013). After binding to its target on the host cell surface, Pre-F undergoes a conformational change during which site Ø is no longer exposed. Pre-F transitions into a transient intermediate conformation, enabling the F protein to insert into the host cell membrane, leading to fusion of the viral and host cell membranes. A final conformational shift results in a more stable and elongated form of the protein (post-fusion, Post-F). Site II and Site IV of the F protein are specific to Post-F, while Site I is present in both the Pre-F and Post-F conformations (McLellan et al., J. Virol. 85(15): 7788-7796. 2011).

Respiratory syncytial virus (RSV) is the leading viral agent causing severe respiratory tract disease worldwide in older adults. There are currently no vaccines available for the prevention of RSV in older adults, and there are no effective antiviral treatments. The economic and clinical burden placed on the healthcare system during the RSV season will remain high until a preventative treatment option becomes available.

As provided herein, mRNA-based vaccines based upon three different RSV proteins have been developed. The F protein designated FD1 corresponds to a wild-type RSV F protein. The F protein designated FD2 corresponds to a soluble RSV F protein lacking the transmembrane domain and cytoplasmic tail and containing a C terminal fibritin trimerization domain (also known as T4 foldon). The F protein designated FD3 corresponds to a pre-fusion RSV F protein.

As used herein, the term “antigenic site Ø” or “site Ø epitope” refers to a site located at the apex of the pre-fusion RSV F trimer, comprising amino acid residues 62-69 and 196-209 of wild-type RSV F, i.e., FD1 or SEQ ID NO: 1: MELLILKANAITTILTAVTFCFASGQNITEEFYQSTCSAVSKGYLSALRTGWYTSVITIELSNIKKNKCN GTDAKVKLIKQELDKYKNAVTELQLLMQSTQATNNRARRELPRFMNYTLNNAKKTNVTLSKKRKRR FLGFLLGVGSAIASGVAVSKVLHLEGEVNKIKSALLSTNKAVVSLSNGVSVLTSKVLDLKNYIDKQLL PIVNKQSCSISNIETVIEFQQKNNRLLEITREFSVNAGVTTPVSTYMLTNSELLSLINDMPITNDQKKL MSNNVQIVRQQSYSIMSIIKEEVLAYVVQLPLYGVIDTPCWKLHTSPLCTTNTKEGSNICLTRTDRG WYCDNAGSVSFFPQAETCKVQSNRVFCDTMNSLTLPSEVNLCNVDIFNPKYDCKIMTSKTDVSSS VITSLGAIVSCYGKTKCTASNKNRGIIKTFSNGCDYVSNKGVDTVSVGNTLYYVNKQEGKSLYVKGE PIINFYDPLVFPSDEFDASISQVNEKINQSLAFIRKSDELLHNVNAGKSTTNIMITTIIIVIIVILLSLIAVGL LLYCKARSTPVTLSKDQLSGINNIAFSN. The F protein designated FD1 corresponds to WT RSV F protein. The site Ø epitope is a binding site for antibodies that have specificity for pre-fusion RSV F, such as D25 and AM14, and binding of antibodies to the site Ø epitope blocks cell-surface attachment of RSV (see, e.g., McLellan et al., Science, 340(6136): 1113-1117, 2013). Recombinant human anti-RSV antibody D25 (Creative Biolabs®; CAT #: PABL-322) and recombinant human anti-RSV antibody AM14 (Creative Biolabs®; CAT #: PABL-321) are each commercially available.

FD2 or SEQ ID NO: 2 is set forth as:
MELLILKANAITTILTAVTFCFASGQNITEEFYQSTCSAVSKGYLSALRTGWYTSVITIELSNIKKNKCN
GTDAKVKLIKQELDKYKNAVTELQLLMQSTQATNNRARRELPRFMNYTLNNAKKTNVTLSKKRKRR
FLGFLLGVGSAIASGVAVSKVLHLEGEVNKIKSALLSTNKAVVSLSNGVSVLTSKVLDLKNYIDKQLL
PIVNKQSCSISNIETVIEFQQKNNRLLEITREFSVNAGVTTPVSTYMLTNSELLSLINDMPITNDQKKL
MSNNVQIVRQQSYSIMSIIKEEVLAYVVQLPLYGVIDTPCWKLHTSPLCTTNTKEGSNICLTRTDRG
WYCDNAGSVSFFPQAETCKVQSNRVFCDTMNSLTLPSEVNLCNVDIFNPKYDCKIMTSKTDVSSS
VITSLGAIVSCYGKTKCTASNKNRGIIKTFSNGCDYVSNKGVDTVSVGNTLYYVNKQEGKSLYVKGE
PIINFYDPLVFPSDEFDASISQVNEKINQSLAFIRKSDELLSAIGGYIPEAPRDGQAYVRKDGEWVLLS
TFL.
FD3 or SEQ ID NO: 3 is set forth as:
MELLILKANAITTILTAVTFCFASGQNITEEFYQSTCSAVSKGYLSALRTGWYTSVITIELSNIKENKCN
GTDAKVKLIKQELDKYKNAVTELQLLMGSGNVGLGGAIASGVAVSKVLHLEGEVNKIKSALLSTNKA
VVSLSNGVSVLTFKVLDLKNYIDKQLLPILNKQSCSISNPETVIEFQQKNNRLLEITREFSVNAGVTTP
VSTYMLTNSELLSLINDMPITNDQKKLMSNNVQIVRQQSYSIMSIIKEEVLAYVVQLPLYGVIDTPCW
KLHTSPLCTTNTKNGSNICLTRTDRGWYCDNAGNVSFFPQAETCKVQSNRVFCDTMNSRTLPSEV
NLCNVDIFNPKYDCKIMTSKTDVSSSVITSLGAIVSCYGKTKCTASNKNRGIIKTFSNGCDYVSNKGV
DTVSVGNTLYYVNKQEGKSLYVKGEPIINFYDPLVFPSDEFDASISQVNELINQSLAFINQSDELLHN
VNAGKSTTNIMITTIIIVIIVILLSLIAVGLLLYCKARSTPVTLSKDQLSGINNIAFSN.

The mRNAs described herein may comprise an open reading frame (ORF) encoding an RSV F protein antigen, at least one 5′ untranslated region (5′ UTR), at least one 3′ untranslated region (3′ UTR), and at least one polyadenylation (poly(A)) sequence. The mRNAs may further comprise a 5′ cap with the following structure:

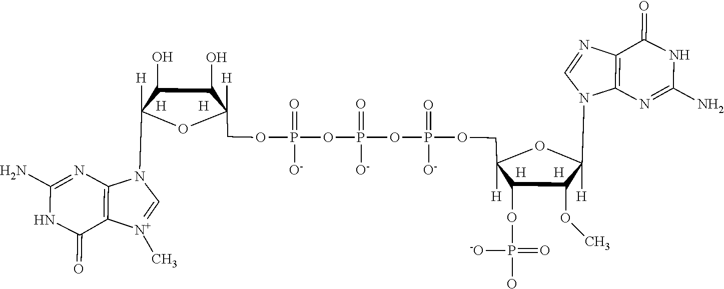

The nucleic acid sequences for each of the mRNA open reading frames (ORFs) encoding the RSV FD1, FD2, and FD3 proteins, respectively, are recited below.

FD1 mRNA ORF:
(SEQ ID NO: 4)
AUGGAAUUGCUGAUCCUCAAAGCGAACGCAAUCACCACUAUCCUCACUGCGGUCACCUUCU
GCUUUGCGAGCGGACAGAACAUCACCGAAGAAUUCUACCAAUCUACUUGCUCCGCCGUGUC
CAAGGGUUACCUGUCCGCCCUGAGGACCGGAUGGUACACUUCCGUGAUUACCAUUGAGUU
GUCGAAUAUCAAGAAGAACAAGUGCAACGGAACCGAUGCUAAGGUCAAGCUGAUCAAGCAG
GAGCUGGACAAGUACAAGAAUGCUGUGACCGAGCUGCAGCUGCUGAUGCAGUCCACUCAAG
CCACCAACAAUCGCGCCCGGCGGGAACUCCCAAGGUUCAUGAACUACACCUUGAACAACGC
CAAGAAAACGAACGUGACCCUGUCCAAGAAGCGCAAGCGCAGAUUCCUUGGCUUCCUUCUG
GGCGUCGGUAGCGCCAUCGCCUCCGGCGUGGCCGUCAGCAAGGUCCUGCACCUCGAGGGA
GAAGUCAACAAGAUUAAGAGCGCCCUGCUGUCCACCAACAAGGCCGUGGUGUCGCUAUCAA
ACGGCGUCAGCGUACUGACCAGCAAAGUGCUGGAUCUCAAGAACUACAUUGAUAAGCAACU
CCUCCCUAUCGUGAAUAAGCAGAGCUGUUCGAUUUCCAACAUCGAGACUGUGAUUGAAUUC
CAGCAGAAGAACAACCGGCUGCUGGAAAUUACCAGAGAAUUCAGCGUGAAUGCCGGAGUCA
CUACCCCCGUGUCCACCUACAUGCUGACAAACUCCGAGCUGCUGAGCCUGAUCAACGAUAU
GCCGAUUACCAACGACCAGAAGAAGCUGAUGUCGAACAACGUGCAGAUCGUGCGCCAGCAG
UCCUACUCAAUCAUGUCGAUCAUCAAGGAAGAGGUCCUGGCCUACGUGGUGCAGCUUCCUC
UGUACGGCGUGAUUGACACUCCGUGUUGGAAACUGCACACUAGUCCCCUGUGCACUACUAA
CACCAAGGAGGGCAGCAAUAUCUGCCUGACUCGGACCGAUAGAGGCUGGUACUGUGAUAAC
GCCGGGUCCGUGUCCUUCUUCCCGCAAGCCGAGACUUGCAAAGUGCAGAGCAACCGGGUG
UUCUGUGACACUAUGAACUCACUGACCUUGCCGAGCGAAGUCAACCUUUGCAACGUGGACA
UCUUUAACCCUAAAUACGACUGCAAGAUCAUGACCUCCAAGACCGACGUGUCGAGCUCAGU
GAUUACUUCGCUGGGAGCCAUUGUGUCCUGCUACGGGAAAACCAAGUGCACGGCCUCAAAC
AAGAACCGGGGUAUCAUUAAGACCUUCUCCAACGGCUGCGACUAUGUGUCCAACAAGGGGG
UGGACACUGUGUCCGUGGGAAACACCUUGUAUUACGUGAACAAGCAGGAGGGAAAGUCCCU
CUACGUGAAGGGCGAACCCAUCAUCAAUUUCUACGACCCGCUCGUGUUCCCCUCCGAUGAA
UUCGACGCAUCCAUCUCACAAGUCAACGAAAAGAUUAACCAGUCCCUGGCUUUCAUUCGCA
AGUCCGACGAACUGCUCCAUAACGUCAACGCUGGAAAGUCCACCACCAACAUCAUGAUCAC
CACGAUCAUUAUUGUGAUCAUCGUCAUCCUGCUGUCACUGAUAGCAGUGGGACUGCUCCUC
UACUGCAAAGCGCGGUCGACCCCAGUGACACUCUCGAAGGACCAGCUGUCCGGGAUCAACA
ACAUCGCGUUUUCGAACUGA.
FD2 mRNA ORF:
(SEQ ID NO: 5)
AUGGAACUCCUGAUCCUGAAGGCCAAUGCUAUCACUACCAUCCUGACUGCCGUCACCUUCU
GCUUCGCCUCCGGACAAAAUAUCACUGAAGAAUUUUACCAAAGCACCUGUAGCGCGGUGUC
CAAGGGAUACCUGAGCGCUCUGAGGACCGGAUGGUACACCAGCGUGAUUACCAUCGAGCU
GAGUAACAUCAAGAAGAACAAGUGCAACGGGACCGAUGCUAAGGUCAAGUUGAUCAAACAA
GAGCUCGACAAGUACAAGAACGCCGUGACUGAGCUGCAGCUGCUGAUGCAGUCAACUCAGG
CCACCAACAACCGGGCCAGACGGGAACUGCCGAGAUUCAUGAACUACACCCUGAACAACGC
CAAAAAGACCAACGUGACCCUGUCCAAGAAGAGAAAGCGCCGGUUCCUGGGUUUCCUGCUU
GGCGUGGGAUCAGCAAUCGCGUCCGGAGUGGCAGUGUCCAAGGUCUUGCACCUCGAGGGC
GAAGUGAACAAGAUCAAGUCCGCGCUUCUGUCGACCAACAAGGCCGUCGUUUCCCUGUCGA
ACGGAGUGUCCGUGCUCACGAGCAAAGUGCUCGACCUGAAGAACUACAUCGACAAACAGCU
GCUGCCCAUCGUCAACAAGCAGAGCUGCAGCAUCUCAAACAUUGAAACCGUGAUCGAGUUC
CAGCAGAAGAACAACCGCCUGCUCGAGAUUACCAGAGAGUUUUCCGUGAACGCCGGCGUGA
CCACCCCGGUGUCGACCUACAUGCUCACAAAUUCGGAACUUCUCUCCCUGAUUAAUGACAU
GCCCAUUACUAACGAUCAGAAAAAGCUGAUGUCGAACAAUGUGCAGAUUGUGCGCCAGCAG
UCCUACUCCAUCAUGUCCAUCAUUAAGGAAGAGGUCCUGGCCUACGUGGUGCAGUUGCCG
CUGUACGGUGUCAUCGAUACCCCCUGCUGGAAGCUCCAUACUUCGCCCCUGUGUACUACCA
ACACCAAGGAAGGCUCCAACAUCUGCCUGACCCGGACGGAUCGCGGCUGGUACUGUGACAA
UGCCGGAUCCGUGUCGUUCUUCCCGCAAGCGGAGACUUGCAAAGUGCAGUCCAACCGGGU
GUUCUGUGACACUAUGAACUCCCUGACUCUGCCGUCCGAAGUCAACCUCUGCAACGUGGAC
AUUUUCAAUCCAAAAUACGACUGCAAGAUAAUGACCUCCAAGACUGACGUGUCAUCGUCCG
UGAUCACAUCUCUGGGAGCCAUUGUCUCCUGCUACGGAAAGACUAAGUGCACCGCGUCGAA
CAAGAACAGGGGCAUUAUCAAGACCUUCAGCAACGGUUGCGACUAUGUGUCCAACAAGGGC
GUGGAUACCGUGUCCGUGGGCAACACCUUGUACUACGUGAACAAGCAGGAGGGGAAGUCC
CUUUAUGUGAAGGGGGAGCCAAUCAUUAACUUUUACGACCCCCUGGUGUUCCCUAGCGACG
AGUUCGACGCCUCAAUCUCUCAAGUCAACGAAAAGAUCAACCAGAGCCUCGCCUUCAUCCG
CAAGUCCGAUGAACUGCUGUCAGCCAUUGGGGGUUACAUCCCUGAGGCCCCUCGGGACGG
ACAGGCAUACGUCCGCAAGGACGGCGAAUGGGUGCUGCUUAGCACCUUCCUCUAA.
FD3 mRNA ORF:
(SEQ ID NO: 6)
AUGGAACUGCUGAUCCUCAAAGCCAACGCAAUCACCACCAUUCUCACCGCUGUGACCUUCU
GCUUCGCAUCGGGGCAGAACAUCACUGAAGAGUUUUACCAGAGCACUUGCAGCGCGGUGU
CAAAGGGUUACCUUUCCGCACUGCGGACCGGAUGGUACACUUCCGUGAUCACCAUUGAGCU
CAGCAACAUCAAGGAAAACAAGUGCAAUGGCACCGACGCCAAGGUCAAGCUGAUCAAACAAG
AACUGGACAAGUACAAGAACGCCGUGACAGAAUUGCAGCUCCUGAUGGGAUCCGGAAACGU
CGGUCUGGGCGGAGCCAUCGCGAGUGGAGUGGCUGUGUCCAAGGUCUUGCACCUCGAGG
GAGAAGUGAACAAGAUCAAGUCCGCGCUGCUGUCAACGAACAAGGCCGUGGUGUCCCUGUC
UAACGGCGUCAGCGUGCUGACGUUCAAGGUCCUGGACCUGAAGAAUUACAUUGACAAGCAG
CUGCUGCCCAUCCUCAACAAGCAAUCCUGCUCCAUCUCCAACCCCGAAACCGUGAUCGAGU
UCCAGCAGAAGAACAACCGCCUGCUGGAAAUUACUCGCGAGUUCUCUGUGAAUGCCGGCGU
GACCACCCCUGUGUCCACCUACAUGCUGACCAACUCCGAGCUUCUCUCCCUUAUCAAUGAC
AUGCCUAUCACGAACGACCAGAAGAAGCUGAUGUCGAACAACGUGCAGAUUGUGCGGCAGC
AGUCAUACAGCAUCAUGUCGAUCAUCAAGGAAGAAGUGCUGGCGUACGUGGUGCAACUCCC
GCUGUACGGCGUCAUCGAUACCCCGUGCUGGAAGCUGCACACCUCGCCUUUGUGUACCAC
CAACACCAAGAACGGAUCCAACAUCUGCUUAACCCGGACUGAUCGGGGUUGGUACUGCGAC
AACGCCGGGAAUGUUUCGUUCUUCCCACAAGCCGAGACUUGUAAAGUGCAGUCAAACAGAG
UGUUCUGUGACACCAUGAACUCGAGAACCCUGCCCAGCGAAGUGAACCUGUGUAACGUCGA
CAUCUUUAACCCAAAAUACGAUUGCAAGAUUAUGACCAGCAAAACCGACGUGUCCUCCUCCG
UGAUAACAAGCCUGGGGGCGAUUGUGUCAUGCUACGGAAAGACUAAGUGCACCGCCUCGAA
CAAGAACCGCGGCAUCAUUAAGACUUUCUCGAAUGGUUGCGACUAUGUGUCCAACAAGGGC
GUGGAUACUGUGUCAGUCGGGAAUACUCUUUACUACGUGAACAAGCAGGAGGGGAAAAGCC
UCUACGUGAAGGGAGAGCCUAUUAUCAACUUUUACGAUCCGCUGGUGUUCCCGUCCGACGA
AUUCGACGCCAGCAUCAGCCAAGUCAACGAGCUGAUUAACCAGUCCCUCGCCUUCAUCAAC
CAAUCCGACGAGCUCCUGCAUAACGUGAACGCCGGAAAGUCCACCACCAACAUCAUGAUCA
CUACUAUUAUCAUCGUGAUCAUCGUCAUCCUGCUGAGCCUGAUUGCUGUGGGCCUGUUGC
UGUAUUGCAAAGCCAGGUCCACCCCGGUCACCCUGUCGAAGGAUCAGCUGUCCGGAAUCAA
CAACAUUGCCUUCUCCAACUAA.

The nucleic acid sequences for each of the DNA templates encoding the RSV FD1, FD2, and FD3 proteins, respectively, are recited below.

FD1 DNA:
(SEQ ID NO: 7)
ATGGAATTGCTGATCCTCAAAGCGAACGCAATCACCACTATCCTCACTGCGGTCACCTTCTGCT
TTGCGAGCGGACAGAACATCACCGAAGAATTCTACCAATCTACTTGCTCCGCCGTGTCCAAGG
GTTACCTGTCCGCCCTGAGGACCGGATGGTACACTTCCGTGATTACCATTGAGTTGTCGAATA
TCAAGAAGAACAAGTGCAACGGAACCGATGCTAAGGTCAAGCTGATCAAGCAGGAGCTGGAC
AAGTACAAGAATGCTGTGACCGAGCTGCAGCTGCTGATGCAGTCCACTCAAGCCACCAACAAT
CGCGCCCGGCGGGAACTCCCAAGGTTCATGAACTACACCTTGAACAACGCCAAGAAAACGAA
CGTGACCCTGTCCAAGAAGCGCAAGCGCAGATTCCTTGGCTTCCTTCTGGGCGTCGGTAGCG
CCATCGCCTCCGGCGTGGCCGTCAGCAAGGTCCTGCACCTCGAGGGAGAAGTCAACAAGATT
AAGAGCGCCCTGCTGTCCACCAACAAGGCCGTGGTGTCGCTATCAAACGGCGTCAGCGTACT
GACCAGCAAAGTGCTGGATCTCAAGAACTACATTGATAAGCAACTCCTCCCTATCGTGAATAAG
CAGAGCTGTTCGATTTCCAACATCGAGACTGTGATTGAATTCCAGCAGAAGAACAACCGGCTG
CTGGAAATTACCAGAGAATTCAGCGTGAATGCCGGAGTCACTACCCCCGTGTCCACCTACATG
CTGACAAACTCCGAGCTGCTGAGCCTGATCAACGATATGCCGATTACCAACGACCAGAAGAAG
CTGATGTCGAACAACGTGCAGATCGTGCGCCAGCAGTCCTACTCAATCATGTCGATCATCAAG
GAAGAGGTCCTGGCCTACGTGGTGCAGCTTCCTCTGTACGGCGTGATTGACACTCCGTGTTG
GAAACTGCACACTAGTCCCCTGTGCACTACTAACACCAAGGAGGGCAGCAATATCTGCCTGAC
TCGGACCGATAGAGGCTGGTACTGTGATAACGCCGGGTCCGTGTCCTTCTTCCCGCAAGCCG
AGACTTGCAAAGTGCAGAGCAACCGGGTGTTCTGTGACACTATGAACTCACTGACCTTGCCGA
GCGAAGTCAACCTTTGCAACGTGGACATCTTTAACCCTAAATACGACTGCAAGATCATGACCTC
CAAGACCGACGTGTCGAGCTCAGTGATTACTTCGCTGGGAGCCATTGTGTCCTGCTACGGGAA
AACCAAGTGCACGGCCTCAAACAAGAACCGGGGTATCATTAAGACCTTCTCCAACGGCTGCGA
CTATGTGTCCAACAAGGGGGTGGACACTGTGTCCGTGGGAAACACCTTGTATTACGTGAACAA
GCAGGAGGGAAAGTCCCTCTACGTGAAGGGCGAACCCATCATCAATTTCTACGACCCGCTCGT
GTTCCCCTCCGATGAATTCGACGCATCCATCTCACAAGTCAACGAAAAGATTAACCAGTCCCTG
GCTTTCATTCGCAAGTCCGACGAACTGCTCCATAACGTCAACGCTGGAAAGTCCACCACCAAC
ATCATGATCACCACGATCATTATTGTGATCATCGTCATCCTGCTGTCACTGATAGCAGTGGGAC
TGCTCCTCTACTGCAAAGCGCGGTCGACCCCAGTGACACTCTCGAAGGACCAGCTGTCCGGG
ATCAACAACATCGCGTTTTCGAACTGA.
FD2 DNA:
(SEQ ID NO: 8)
ATGGAACTCCTGATCCTGAAGGCCAATGCTATCACTACCATCCTGACTGCCGTCACCTTCTGCT
TCGCCTCCGGACAAAATATCACTGAAGAATTTTACCAAAGCACCTGTAGCGCGGTGTCCAAGG
GATACCTGAGCGCTCTGAGGACCGGATGGTACACCAGCGTGATTACCATCGAGCTGAGTAAC
ATCAAGAAGAACAAGTGCAACGGGACCGATGCTAAGGTCAAGTTGATCAAACAAGAGCTCGAC
AAGTACAAGAACGCCGTGACTGAGCTGCAGCTGCTGATGCAGTCAACTCAGGCCACCAACAA
CCGGGCCAGACGGGAACTGCCGAGATTCATGAACTACACCCTGAACAACGCCAAAAAGACCA
ACGTGACCCTGTCCAAGAAGAGAAAGCGCCGGTTCCTGGGTTTCCTGCTTGGCGTGGGATCA
GCAATCGCGTCCGGAGTGGCAGTGTCCAAGGTCTTGCACCTCGAGGGCGAAGTGAACAAGAT
CAAGTCCGCGCTTCTGTCGACCAACAAGGCCGTCGTTTCCCTGTCGAACGGAGTGTCCGTGC
TCACGAGCAAAGTGCTCGACCTGAAGAACTACATCGACAAACAGCTGCTGCCCATCGTCAACA
AGCAGAGCTGCAGCATCTCAAACATTGAAACCGTGATCGAGTTCCAGCAGAAGAACAACCGCC
TGCTCGAGATTACCAGAGAGTTTTCCGTGAACGCCGGCGTGACCACCCCGGTGTCGACCTAC
ATGCTCACAAATTCGGAACTTCTCTCCCTGATTAATGACATGCCCATTACTAACGATCAGAAAA
AGCTGATGTCGAACAATGTGCAGATTGTGCGCCAGCAGTCCTACTCCATCATGTCCATCATTAA
GGAAGAGGTCCTGGCCTACGTGGTGCAGTTGCCGCTGTACGGTGTCATCGATACCCCCTGCT
GGAAGCTCCATACTTCGCCCCTGTGTACTACCAACACCAAGGAAGGCTCCAACATCTGCCTGA
CCCGGACGGATCGCGGCTGGTACTGTGACAATGCCGGATCCGTGTCGTTCTTCCCGCAAGCG
GAGACTTGCAAAGTGCAGTCCAACCGGGTGTTCTGTGACACTATGAACTCCCTGACTCTGCCG
TCCGAAGTCAACCTCTGCAACGTGGACATTTTCAATCCAAAATACGACTGCAAGATAATGACCT
CCAAGACTGACGTGTCATCGTCCGTGATCACATCTCTGGGAGCCATTGTCTCCTGCTACGGAA
AGACTAAGTGCACCGCGTCGAACAAGAACAGGGGCATTATCAAGACCTTCAGCAACGGTTGC
GACTATGTGTCCAACAAGGGCGTGGATACCGTGTCCGTGGGCAACACCTTGTACTACGTGAAC
AAGCAGGAGGGGAAGTCCCTTTATGTGAAGGGGGAGCCAATCATTAACTTTTACGACCCCCTG
GTGTTCCCTAGCGACGAGTTCGACGCCTCAATCTCTCAAGTCAACGAAAAGATCAACCAGAGC
CTCGCCTTCATCCGCAAGTCCGATGAACTGCTGTCAGCCATTGGGGGTTACATCCCTGAGGCC
CCTCGGGACGGACAGGCATACGTCCGCAAGGACGGCGAATGGGTGCTGCTTAGCACCTTCCT
CTAA.
FD3 DNA:
(SEQ ID NO: 9)
ATGGAACTGCTGATCCTCAAAGCCAACGCAATCACCACCATTCTCACCGCTGTGACCTTCTGC
TTCGCATCGGGGCAGAACATCACTGAAGAGTTTTACCAGAGCACTTGCAGCGCGGTGTCAAAG
GGTTACCTTTCCGCACTGCGGACCGGATGGTACACTTCCGTGATCACCATTGAGCTCAGCAAC
ATCAAGGAAAACAAGTGCAATGGCACCGACGCCAAGGTCAAGCTGATCAAACAAGAACTGGAC
AAGTACAAGAACGCCGTGACAGAATTGCAGCTCCTGATGGGATCCGGAAACGTCGGTCTGGG
CGGAGCCATCGCGAGTGGAGTGGCTGTGTCCAAGGTCTTGCACCTCGAGGGAGAAGTGAACA
AGATCAAGTCCGCGCTGCTGTCAACGAACAAGGCCGTGGTGTCCCTGTCTAACGGCGTCAGC
GTGCTGACGTTCAAGGTCCTGGACCTGAAGAATTACATTGACAAGCAGCTGCTGCCCATCCTC
AACAAGCAATCCTGCTCCATCTCCAACCCCGAAACCGTGATCGAGTTCCAGCAGAAGAACAAC
CGCCTGCTGGAAATTACTCGCGAGTTCTCTGTGAATGCCGGCGTGACCACCCCTGTGTCCACC
TACATGCTGACCAACTCCGAGCTTCTCTCCCTTATCAATGACATGCCTATCACGAACGACCAGA
AGAAGCTGATGTCGAACAACGTGCAGATTGTGCGGCAGCAGTCATACAGCATCATGTCGATCA
TCAAGGAAGAAGTGCTGGCGTACGTGGTGCAACTCCCGCTGTACGGCGTCATCGATACCCCG
TGCTGGAAGCTGCACACCTCGCCTTTGTGTACCACCAACACCAAGAACGGATCCAACATCTGC
TTAACCCGGACTGATCGGGGTTGGTACTGCGACAACGCCGGGAATGTTTCGTTCTTCCCACAA
GCCGAGACTTGTAAAGTGCAGTCAAACAGAGTGTTCTGTGACACCATGAACTCGAGAACCCTG
CCCAGCGAAGTGAACCTGTGTAACGTCGACATCTTTAACCCAAAATACGATTGCAAGATTATGA
CCAGCAAAACCGACGTGTCCTCCTCCGTGATAACAAGCCTGGGGGCGATTGTGTCATGCTAC
GGAAAGACTAAGTGCACCGCCTCGAACAAGAACCGCGGCATCATTAAGACTTTCTCGAATGGT
TGCGACTATGTGTCCAACAAGGGCGTGGATACTGTGTCAGTCGGGAATACTCTTTACTACGTG
AACAAGCAGGAGGGGAAAAGCCTCTACGTGAAGGGAGAGCCTATTATCAACTTTTACGATCCG
CTGGTGTTCCCGTCCGACGAATTCGACGCCAGCATCAGCCAAGTCAACGAGCTGATTAACCAG
TCCCTCGCCTTCATCAACCAATCCGACGAGCTCCTGCATAACGTGAACGCCGGAAAGTCCACC
ACCAACATCATGATCACTACTATTATCATCGTGATCATCGTCATCCTGCTGAGCCTGATTGCTG
TGGGCCTGTTGCTGTATTGCAAAGCCAGGTCCACCCCGGTCACCCTGTCGAAGGATCAGCTG
TCCGGAATCAACAACATTGCCTTCTCCAACTAA.

The nucleic acid sequences for the 5′UTR and 3′UTR are recited below.

5′UTR:
(SEQ ID NO: 10)
GGACAGAUCGCCUGGAGACGCCAUCCACGCUGUUUUGACCUCCAUAGAA
GACACCGGGACCGAUCCAGCCUCCGCGGCCGGGAACGGUGCAUUGGAAC
GCGGAUUCCCCGUGCCAAGAGUGACUCACCGUCCUUGACACG.
3′UTR:
(SEQ ID NO: 11)
CGGGUGGCAUCCCUGUGACCCCUCCCCAGUGCCUCUCCUGGCCCUGGAA
GUUGCCACUCCAGUGCCCACCAGCCUUGUCCUAAUAAAAUUAAGUUGCA
UC.

The nucleic acid sequences for each of the full-length mRNA encoding the RSV FD1, FD2, and FD3 proteins are recited below.

FD1 mRNA:
(SEQ ID NO: 12)
GGACAGAUCGCCUGGAGACGCCAUCCACGCUGUUUUGACCUCCAUAGAAGACACCGGGACC
GAUCCAGCCUCCGCGGCCGGGAACGGUGCAUUGGAACGCGGAUUCCCCGUGCCAAGAGUG
ACUCACCGUCCUUGACACGAUGGAAUUGCUGAUCCUCAAAGCGAACGCAAUCACCACUAUC
CUCACUGCGGUCACCUUCUGCUUUGCGAGCGGACAGAACAUCACCGAAGAAUUCUACCAAU
CUACUUGCUCCGCCGUGUCCAAGGGUUACCUGUCCGCCCUGAGGACCGGAUGGUACACUU
CCGUGAUUACCAUUGAGUUGUCGAAUAUCAAGAAGAACAAGUGCAACGGAACCGAUGCUAA
GGUCAAGCUGAUCAAGCAGGAGCUGGACAAGUACAAGAAUGCUGUGACCGAGCUGCAGCUG
CUGAUGCAGUCCACUCAAGCCACCAACAAUCGCGCCCGGCGGGAACUCCCAAGGUUCAUGA
ACUACACCUUGAACAACGCCAAGAAAACGAACGUGACCCUGUCCAAGAAGCGCAAGCGCAGA
UUCCUUGGCUUCCUUCUGGGCGUCGGUAGCGCCAUCGCCUCCGGCGUGGCCGUCAGCAAG
GUCCUGCACCUCGAGGGAGAAGUCAACAAGAUUAAGAGCGCCCUGCUGUCCACCAACAAGG
CCGUGGUGUCGCUAUCAAACGGCGUCAGCGUACUGACCAGCAAAGUGCUGGAUCUCAAGAA
CUACAUUGAUAAGCAACUCCUCCCUAUCGUGAAUAAGCAGAGCUGUUCGAUUUCCAACAUC
GAGACUGUGAUUGAAUUCCAGCAGAAGAACAACCGGCUGCUGGAAAUUACCAGAGAAUUCA
GCGUGAAUGCCGGAGUCACUACCCCCGUGUCCACCUACAUGCUGACAAACUCCGAGCUGCU
GAGCCUGAUCAACGAUAUGCCGAUUACCAACGACCAGAAGAAGCUGAUGUCGAACAACGUG
CAGAUCGUGCGCCAGCAGUCCUACUCAAUCAUGUCGAUCAUCAAGGAAGAGGUCCUGGCCU
ACGUGGUGCAGCUUCCUCUGUACGGCGUGAUUGACACUCCGUGUUGGAAACUGCACACUA
GUCCCCUGUGCACUACUAACACCAAGGAGGGCAGCAAUAUCUGCCUGACUCGGACCGAUAG
AGGCUGGUACUGUGAUAACGCCGGGUCCGUGUCCUUCUUCCCGCAAGCCGAGACUUGCAA
AGUGCAGAGCAACCGGGUGUUCUGUGACACUAUGAACUCACUGACCUUGCCGAGCGAAGUC
AACCUUUGCAACGUGGACAUCUUUAACCCUAAAUACGACUGCAAGAUCAUGACCUCCAAGAC
CGACGUGUCGAGCUCAGUGAUUACUUCGCUGGGAGCCAUUGUGUCCUGCUACGGGAAAAC
CAAGUGCACGGCCUCAAACAAGAACCGGGGUAUCAUUAAGACCUUCUCCAACGGCUGCGAC
UAUGUGUCCAACAAGGGGGUGGACACUGUGUCCGUGGGAAACACCUUGUAUUACGUGAACA
AGCAGGAGGGAAAGUCCCUCUACGUGAAGGGCGAACCCAUCAUCAAUUUCUACGACCCGCU
CGUGUUCCCCUCCGAUGAAUUCGACGCAUCCAUCUCACAAGUCAACGAAAAGAUUAACCAG
UCCCUGGCUUUCAUUCGCAAGUCCGACGAACUGCUCCAUAACGUCAACGCUGGAAAGUCCA
CCACCAACAUCAUGAUCACCACGAUCAUUAUUGUGAUCAUCGUCAUCCUGCUGUCACUGAU
AGCAGUGGGACUGCUCCUCUACUGCAAAGCGCGGUCGACCCCAGUGACACUCUCGAAGGA
CCAGCUGUCCGGGAUCAACAACAUCGCGUUUUCGAACUGACGGGUGGCAUCCCUGUGACC
CCUCCCCAGUGCCUCUCCUGGCCCUGGAAGUUGCCACUCCAGUGCCCACCAGCCUUGUCC
UAAUAAAAUUAAGUUGCAUC.
FD2 mRNA:
(SEQ ID NO: 13)
GGACAGAUCGCCUGGAGACGCCAUCCACGCUGUUUUGACCUCCAUAGAAGACACCGGGACC
GAUCCAGCCUCCGCGGCCGGGAACGGUGCAUUGGAACGCGGAUUCCCCGUGCCAAGAGUG
ACUCACCGUCCUUGACACGAUGGAACUCCUGAUCCUGAAGGCCAAUGCUAUCACUACCAUC
CUGACUGCCGUCACCUUCUGCUUCGCCUCCGGACAAAAUAUCACUGAAGAAUUUUACCAAA
GCACCUGUAGCGCGGUGUCCAAGGGAUACCUGAGCGCUCUGAGGACCGGAUGGUACACCA
GCGUGAUUACCAUCGAGCUGAGUAACAUCAAGAAGAACAAGUGCAACGGGACCGAUGCUAA
GGUCAAGUUGAUCAAACAAGAGCUCGACAAGUACAAGAACGCCGUGACUGAGCUGCAGCUG
CUGAUGCAGUCAACUCAGGCCACCAACAACCGGGCCAGACGGGAACUGCCGAGAUUCAUGA
ACUACACCCUGAACAACGCCAAAAAGACCAACGUGACCCUGUCCAAGAAGAGAAAGCGCCGG
UUCCUGGGUUUCCUGCUUGGCGUGGGAUCAGCAAUCGCGUCCGGAGUGGCAGUGUCCAAG
GUCUUGCACCUCGAGGGCGAAGUGAACAAGAUCAAGUCCGCGCUUCUGUCGACCAACAAGG
CCGUCGUUUCCCUGUCGAACGGAGUGUCCGUGCUCACGAGCAAAGUGCUCGACCUGAAGA
ACUACAUCGACAAACAGCUGCUGCCCAUCGUCAACAAGCAGAGCUGCAGCAUCUCAAACAUU
GAAACCGUGAUCGAGUUCCAGCAGAAGAACAACCGCCUGCUCGAGAUUACCAGAGAGUUUU
CCGUGAACGCCGGCGUGACCACCCCGGUGUCGACCUACAUGCUCACAAAUUCGGAACUUCU
CUCCCUGAUUAAUGACAUGCCCAUUACUAACGAUCAGAAAAAGCUGAUGUCGAACAAUGUG
CAGAUUGUGCGCCAGCAGUCCUACUCCAUCAUGUCCAUCAUUAAGGAAGAGGUCCUGGCCU
ACGUGGUGCAGUUGCCGCUGUACGGUGUCAUCGAUACCCCCUGCUGGAAGCUCCAUACUU
CGCCCCUGUGUACUACCAACACCAAGGAAGGCUCCAACAUCUGCCUGACCCGGACGGAUCG
CGGCUGGUACUGUGACAAUGCCGGAUCCGUGUCGUUCUUCCCGCAAGCGGAGACUUGCAA
AGUGCAGUCCAACCGGGUGUUCUGUGACACUAUGAACUCCCUGACUCUGCCGUCCGAAGU
CAACCUCUGCAACGUGGACAUUUUCAAUCCAAAAUACGACUGCAAGAUAAUGACCUCCAAGA
CUGACGUGUCAUCGUCCGUGAUCACAUCUCUGGGAGCCAUUGUCUCCUGCUACGGAAAGA
CUAAGUGCACCGCGUCGAACAAGAACAGGGGCAUUAUCAAGACCUUCAGCAACGGUUGCGA
CUAUGUGUCCAACAAGGGCGUGGAUACCGUGUCCGUGGGCAACACCUUGUACUACGUGAA
CAAGCAGGAGGGGAAGUCCCUUUAUGUGAAGGGGGAGCCAAUCAUUAACUUUUACGACCCC
CUGGUGUUCCCUAGCGACGAGUUCGACGCCUCAAUCUCUCAAGUCAACGAAAAGAUCAACC
AGAGCCUCGCCUUCAUCCGCAAGUCCGAUGAACUGCUGUCAGCCAUUGGGGGUUACAUCC
CUGAGGCCCCUCGGGACGGACAGGCAUACGUCCGCAAGGACGGCGAAUGGGUGCUGCUUA
GCACCUUCCUCUAACGGGUGGCAUCCCUGUGACCCCUCCCCAGUGCCUCUCCUGGCCCUG
GAAGUUGCCACUCCAGUGCCCACCAGCCUUGUCCUAAUAAAAUUAAGUUGCAUC.
FD3 mRNA:
(SEQ ID NO: 14)
GGACAGAUCGCCUGGAGACGCCAUCCACGCUGUUUUGACCUCCAUAGAAGACACCGGGACC
GAUCCAGCCUCCGCGGCCGGGAACGGUGCAUUGGAACGCGGAUUCCCCGUGCCAAGAGUG
ACUCACCGUCCUUGACACGAUGGAACUGCUGAUCCUCAAAGCCAACGCAAUCACCACCAUU
CUCACCGCUGUGACCUUCUGCUUCGCAUCGGGGCAGAACAUCACUGAAGAGUUUUACCAGA
GCACUUGCAGCGCGGUGUCAAAGGGUUACCUUUCCGCACUGCGGACCGGAUGGUACACUU
CCGUGAUCACCAUUGAGCUCAGCAACAUCAAGGAAAACAAGUGCAAUGGCACCGACGCCAA
GGUCAAGCUGAUCAAACAAGAACUGGACAAGUACAAGAACGCCGUGACAGAAUUGCAGCUC
CUGAUGGGAUCCGGAAACGUCGGUCUGGGCGGAGCCAUCGCGAGUGGAGUGGCUGUGUC
CAAGGUCUUGCACCUCGAGGGAGAAGUGAACAAGAUCAAGUCCGCGCUGCUGUCAACGAAC
AAGGCCGUGGUGUCCCUGUCUAACGGCGUCAGCGUGCUGACGUUCAAGGUCCUGGACCUG
AAGAAUUACAUUGACAAGCAGCUGCUGCCCAUCCUCAACAAGCAAUCCUGCUCCAUCUCCAA
CCCCGAAACCGUGAUCGAGUUCCAGCAGAAGAACAACCGCCUGCUGGAAAUUACUCGCGAG
UUCUCUGUGAAUGCCGGCGUGACCACCCCUGUGUCCACCUACAUGCUGACCAACUCCGAGC
UUCUCUCCCUUAUCAAUGACAUGCCUAUCACGAACGACCAGAAGAAGCUGAUGUCGAACAA
CGUGCAGAUUGUGCGGCAGCAGUCAUACAGCAUCAUGUCGAUCAUCAAGGAAGAAGUGCUG
GCGUACGUGGUGCAACUCCCGCUGUACGGCGUCAUCGAUACCCCGUGCUGGAAGCUGCAC
ACCUCGCCUUUGUGUACCACCAACACCAAGAACGGAUCCAACAUCUGCUUAACCCGGACUG
AUCGGGGUUGGUACUGCGACAACGCCGGGAAUGUUUCGUUCUUCCCACAAGCCGAGACUU
GUAAAGUGCAGUCAAACAGAGUGUUCUGUGACACCAUGAACUCGAGAACCCUGCCCAGCGA
AGUGAACCUGUGUAACGUCGACAUCUUUAACCCAAAAUACGAUUGCAAGAUUAUGACCAGCA
AAACCGACGUGUCCUCCUCCGUGAUAACAAGCCUGGGGGCGAUUGUGUCAUGCUACGGAAA
GACUAAGUGCACCGCCUCGAACAAGAACCGCGGCAUCAUUAAGACUUUCUCGAAUGGUUGC
GACUAUGUGUCCAACAAGGGCGUGGAUACUGUGUCAGUCGGGAAUACUCUUUACUACGUGA
ACAAGCAGGAGGGGAAAAGCCUCUACGUGAAGGGAGAGCCUAUUAUCAACUUUUACGAUCC
GCUGGUGUUCCCGUCCGACGAAUUCGACGCCAGCAUCAGCCAAGUCAACGAGCUGAUUAAC
CAGUCCCUCGCCUUCAUCAACCAAUCCGACGAGCUCCUGCAUAACGUGAACGCCGGAAAGU
CCACCACCAACAUCAUGAUCACUACUAUUAUCAUCGUGAUCAUCGUCAUCCUGCUGAGCCU
GAUUGCUGUGGGCCUGUUGCUGUAUUGCAAAGCCAGGUCCACCCCGGUCACCCUGUCGAA
GGAUCAGCUGUCCGGAAUCAACAACAUUGCCUUCUCCAACUAACGGGUGGCAUCCCUGUGA
CCCCUCCCCAGUGCCUCUCCUGGCCCUGGAAGUUGCCACUCCAGUGCCCACCAGCCUUGU
CCUAAUAAAAUUAAGUUGCAUC.

One aspect of the present disclosure is directed to methods of eliciting or stimulating an immune response against RSV in a subject. Another aspect of the present disclosure is related to methods of preventing an RSV infection or reducing one or more symptoms of an RSV infection in a subject. The methods may include administering or providing an RNA (e.g., an mRNA) RSV vaccine to the subject. The RNA RSV vaccine may include an mRNA, wherein the mRNA includes an ORF encoding an RSV F protein antigen or a portion of an RSV F protein antigen.

In certain embodiments, a prophylactically effective amount of the RNA RSV vaccine can be administered (e.g., by a medical practitioner) to the subject. In certain embodiments, the RSV F protein antigen can include an amino acid sequence having at least 80%, 85%, 90%, 91%, 92%, 93%, 94%, 95%, 96%, 97%, 98%, 99%, 99.5% or 100% identity to SEQ ID NO: 3. In other embodiments, the RSV F protein antigen is encoded by an RNA sequence (e.g., an mRNA sequence) having at least 80%, 85%, 90%, 91%, 92%, 93%, 94%, 95%, 96%, 97%, 98%, 99%, 99.5% or 100% identity to SEQ ID NO: 14.

Another aspect of the present disclosure is directed to methods of eliciting or stimulating an immune response against RSV in a subject, wherein the methods may include selecting a subject that is at least 60 years of age and administering to the subject a prophylactically effective amount of an RNA RSV vaccine. Another aspect of the disclosure is directed to methods of preventing an RSV infection or reducing one or more symptoms of an RSV infection in a subject, wherein the methods may include selecting a subject that is at least 60 years of age and administering to the subject a prophylactically effective amount of an RNA RSV vaccine. The step of selecting a subject that is 60 years of age or older may be performed by a health care worker (e.g., any one or more of a physician, a physician assistant, a nurse, a pharmacist, a pharmacy technician, a medical technician, and the like) or may be performed by the subject themself, i.e., self-selected. The selected subject can then be administered a prophylactically effective amount of an RNA RSV vaccine of the disclosure.

Another aspect of the present disclosure is directed to RNA (e.g., mRNA) RSV vaccines for use in eliciting or stimulating an immune response against RSV in a subject. Another aspect of the present disclosure is directed to RNA (e.g., mRNA) RSV vaccines for use in preventing RSV infection or reducing one or more symptoms of an RSV infection in a subject. In certain embodiments, symptoms of an RSV infection include, but are not limited to, acute respiratory disease (ARD), medically attended acute respiratory disease (MAARD), severe ARD, non-medically attended lower respiratory tract disease (LRTD), medically attended LRTD, congestion, runny nose, cough, fever, sore throat, headache, pneumonia, bronchiolitis, bronchopneumonia, and tracheobronchitis. RSV infection can be confirmed by laboratory testing, e.g., by RT-PCR, ELISA, and the like.

As used herein, “ARD” refers to RSV infection that includes any respiratory symptoms including nasal congestion, sore throat, hoarseness, new or worsening cough, sputum production, and dyspnea with or without fever.

As used herein, “severe ARD” refers to RSV infection that includes an acute respiratory disease with history of fever or measured fever of ≥38° C. and cough with onset within the last 10 days that requires hospitalization.

As used herein, “LRTD” refers to RSV infection that includes ARD with one or more symptoms of lower respiratory tract illness, including, but not limited to, affection of the lower respiratory tract: trachea, bronchi, and lungs, which could be combined, e.g., bronchopneumonia and/or tracheobronchitis, with 10 days of ARD symptom onset.

As used herein, “LRTD medically attended” refers to RSV infection that includes ARD with one or more symptoms of lower respiratory tract illness, including, but not limited to, affection of the lower respiratory tract: trachea, bronchi, and lungs, which could be combined, e.g., bronchopneumonia and/or tracheobronchitis, with 10 days of ARD symptom onset, seeking medical attention (e.g., an emergency room visit, hospitalization, or an outpatient clinic visit).

As used herein, “reducing one or more symptoms of an RSV infection” refers to a reduction of one or more symptoms and/or a reduction in viral load by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100%, in a subject vaccinated with an RSV vaccine of the disclosure relative to an unvaccinated subject.

In certain embodiments, an RSV vaccine described herein may be administered to a subject by a method of administration that includes, but is not limited to, intradermal, intramuscular, intraperitoneal, intravenous, subcutaneous, intranasal, intra-tracheal, epidural, and oral routes of administration. In certain embodiments, administration is intramuscular in an arm muscle or in a leg muscle of a subject. In certain embodiments, administration is in the deltoid muscle of the upper arm of a subject. An RSV vaccine described herein can be delivered intramuscularly with a standard needle and syringe or delivered by any other suitable injection device.

In certain embodiments, an RSV vaccine described herein may be administered to a subject by a method of administration that includes skin injection, e.g., in the epidermis, the dermis or the hypodermis of the skin. In some embodiments, the RSV vaccine described herein is provided in a device suitable for skin injection, such as a needle (e.g., an epidermic, dermic, or hypodermic needle), a needle free device, a microneedle device, or a microprojection array device. Examples of microneedle or microprojection array devices suitable for the skin injection according to the invention are described in US20230270842A1, US20220339416A1, US20210085598A1, US20200246450A1, US20220143376A1, US20180264244A1, US20180263641A1, and US20110245776A1.

In certain embodiments, the RSV RNA vaccine composition is formulated to exhibit reduced amounts of ionizable lipid-mRNA adduct impurities (e.g., aldehyde-mRNA adduct impurities) which may form due to covalent modification of mRNA by reactive species (e.g., secondary amines or reactive aldehydes) produced by degradation of the ionizable lipid component of an LNP (see Packer et al., “A Novel mechanism for the loss of mRNA activity in lipid nanoparticle delivery systems.” Nature Communications, (2021) 12:6777). In some embodiments, the vaccine composition comprises less than about 10% (e.g., less than about 10%, less than about 5%, less than about 1%, less than about 0.1%, less than about 0.05%, less than about 0.01%, or less than 0.001%) of the mRNA in the form of an adduct impurity, as measured by reverse phase ion pair high performance liquid chromatography (RP-IP HPLC). In some embodiments, the amount of adduct impurity in an LNP composition increases at an average rate of less than 2%, less than 1%, less than 0.5%, or less than 0.2% per day when stored at a temperature at about 25° C. or below. In some embodiments, the amount of adduct does not substantially increase when stored at a temperature at about 25° C. or below (e.g., does not increase by more than 0.05%, more than 0.01%, more than 0.005%, or more than 0.001%).

In some embodiments, the buffer or pH of the RSV RNA vaccine composition can be adjusted to reduce the amount of adduct impurity formed in the LNP composition (e.g., to inhibit ionizable lipid decomposition). For example, some embodiments may comprise a composition with a TRIS (tris(hydroxymethyl)aminomethane) buffer at a concentration of about 10 mM or more, such as a concentration of about 20 mM, about 30 mM, about 50 mM, about 60 mM, about 75 mM, about 100 mM, about 120 mM, or about 150 mM TRIS buffer. In some embodiments, the composition comprises from about 10 mM to about 150 mM TRIS, such as from about 15 mM to about 120 mM TRIS or about 20 mM to about 100 mM TRIS. In some embodiments, the composition does not contain a PBS buffer. In some embodiments, the composition is at a pH of from about 6.5 to about 9.0, such as about 7-8, about 7-7.5, about 7.4, or about 7.5.

In some embodiments, an RSV RNA vaccine described herein (e.g., an mRNA vaccine composition) comprises a unit dosage volume of about 0.3 mL, about 0.35 mL, about 0.4 mL, about 0.45 mL, about 0.5 mL, about 0.55 mL, about 0.6 mL, about 0.65 mL, or about 0.7 mL and a pharmaceutically acceptable carrier(s), diluent(s), and/or excipient(s). In some embodiments, an RSV RNA vaccine described herein (e.g., an mRNA vaccine composition) is administered to a subject in a volume of about 0.3 mL, about 0.35 mL, about 0.4 mL, about 0.45 mL, about 0.5 mL, about 0.55 mL, about 0.6 mL, about 0.65 mL, or about 0.7 mL.

In some embodiments, an RSV vaccine described herein (e.g., an mRNA vaccine composition) is administered to a subject at a dose of about 5 μg to about 400 μg, about 5 μg to about 300 μg, about 5 μg to about 200 μg, about 5 μg to about 100 μg, or about 5 μg to about 15 μg to vaccinate the subject, wherein the μg consists of the amount of mRNA formulated in a lipid nanoparticle (LNP), not including any diluent, etc. In some embodiments, an RSV vaccine described herein (e.g., an mRNA vaccine composition) is administered to a subject at a dose of about 5 μg to about 160 μg, about 5 μg to about 120 μg, about 10 μg to about 80 μg, about 10 μg to about 60 μg, or about 20 μg to about 40 μg to vaccinate the subject, wherein the μg consists of the amount of mRNA formulated in an LNP, not including any diluent, etc. In some embodiments, an RSV vaccine described herein (e.g., an mRNA vaccine composition) is administered to a subject at a dose of about 45 μg to about 130 μg, about 50 μg to about 120 μg, about 55 μg to about 110 μg, about 60 μg to about 100 μg, or about 65 μg to about 95 μg to vaccinate the subject, wherein the μg consists of the amount of mRNA formulated in an LNP, not including any diluent, etc.

In some embodiments, an RSV vaccine described herein (e.g., an mRNA vaccine composition) is administered to a subject in a dose of about 25 μg, about 50 μg, about 100 μg, about 110 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, or about 1000 μg, wherein the μg consists of the amount of mRNA formulated in an LNP, not including any diluent, etc. In some embodiments, an RSV vaccine described herein (e.g., an mRNA vaccine composition) is administered to a subject in a dose of about 10 μg, about 30 μg, about 75 μg, or about 110 μg, wherein the μg consists of the amount of mRNA formulated in an LNP, not including any diluent, etc.

In some embodiments 10 micrograms of an RSV RNA vaccine composition is administered to a subject in a 0.5 mL dose. In some embodiments 30 micrograms of an RSV RNA vaccine composition is administered to a subject in a 0.5 mL dose. In some embodiments 75 micrograms of an RSV RNA vaccine composition is administered to a subject in a 0.5 mL dose. The μg consists of the amount of mRNA formulated in an LN P, not including any diluent, etc.

In some embodiments, an RSV RNA vaccine composition (e.g., mRNA vaccine composition) for use in a method of vaccinating a subject is administered to the subject in a single dose. In some embodiments, an RSV RNA vaccine composition (e.g., mRNA vaccine composition) for use in a method of vaccinating a subject is administered to the subject as two dosages, e.g., an initial dose and a booster dose. In some embodiments, an RSV RNA vaccine composition (e.g., mRNA vaccine composition) for use in a method of vaccinating a subject is administered to the subject as three or more dosages, e.g., an initial dose and as two or more booster doses (e.g., a first booster dose, a second booster dose, etc.).

In some embodiments, an RSV RNA vaccine composition (e.g., mRNA vaccine composition) for use in a method of vaccinating a subject is administered as an initial dose of the RSV vaccine and a booster dose of the RSV vaccine wherein the initial dose and the booster are spaced temporally.

In certain embodiments, the booster dose is administered to the subject about 1 months to about 24 months, about 2 months to about 23 months, about 3 months to about 22 months, about 4 months to about 21 months, about 5 months to about 20 months, about 6 months to about 19 months, about 7 months to about 18 months, about 8 months to about 17 months, about 9 months to about 16 months, about 10 months to about 15 months, about 10 months to about 14 months, about 11 months to about 14 months, or about 11 months to about 13 months after the initial dose.

In certain embodiments, the booster dose is administered to the subject at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months after the initial dose.

In some embodiments, an RSV RNA vaccine composition (e.g., mRNA vaccine composition) for use in a method of vaccinating a subject is administered to a subject as an initial dose of the RSV vaccine and one or more booster doses of the RSV vaccine.

In certain embodiments, a booster dose is administered to the subject about 1 months to about 24 months, about 2 months to about 23 months, about 3 months to about 22 months, about 4 months to about 21 months, about 5 months to about 20 months, about 6 months to about 19 months, about 7 months to about 18 months, about 8 months to about 17 months, about 9 months to about 16 months, about 10 months to about 15 months, about 10 months to about 14 months, about 11 months to about 14 months, or about 11 months to about 13 months after a preceding initial dose or after a preceding booster dose.

In certain embodiments, the booster dose is administered to the subject at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months after a preceding initial dose or after a preceding booster dose.

### III. Pharmaceutical Compositions

RNA purified according to this disclosure can be useful as a component in pharmaceutical compositions, for example, for use as a vaccine, e.g., an RSV RNA vaccine. These compositions will typically include RNA and a pharmaceutically acceptable carrier. A pharmaceutical composition of the present disclosure can also include a delivery system for the RNA, such as a liposome, an oil-in-water emulsion, or a microparticle. In some embodiments, the pharmaceutical composition comprises a lipid nanoparticle (LNP). In certain embodiments, the composition comprises an antigen-encoding nucleic acid molecule encapsulated within an LNP.

In some embodiments, an RSV RNA vaccine composition comprises a pharmaceutically acceptable carrier, diluent, excipient, and/or LNP and excludes adjuvant. In some embodiments, the vaccine composition comprises a pharmaceutically acceptable carrier, diluent, excipient, and/or LNP and includes one or more adjuvants. In some embodiments, a carrier, diluent, excipient, and/or LNP comprises buffered saline. In some embodiments, a carrier, diluent, excipient, and/or LNP comprises octylphenol ethoxylate (Triton X-100). In some embodiments, a carrier, diluent, excipient, and/or LNP comprises buffered saline and octylphenol ethoxylate (Triton X-100). In some embodiments, a carrier, diluent, excipient, and/or LNP comprises sodium chloride. In some embodiments, a carrier, diluent, excipient, and/or LNP comprises sodium phosphate. In some embodiments, a carrier, diluent, excipient, and/or LNP comprises dibasic sodium phosphate. In some embodiments, a carrier, diluent, excipient, and/or LNP comprises water. In some embodiments, a carrier, diluent, excipient, and/or LNP comprises formaldehyde. In some embodiments, a carrier, diluent, excipient, and/or LNP comprises ovalbumin. In some embodiments, a carrier, diluent, excipient, and/or LNP comprises sodium chloride, sodium phosphate (monobasic, dibasic, or both), and water. In some embodiments, a carrier, diluent, excipient, and/or LNP comprises sodium chloride, sodium phosphate (monobasic, dibasic, or both), water, formaldehyde, ovalbumin, and Triton X-100.

In certain embodiments, compositions will be in aqueous form when administered but may be stored in a non-liquid form and resuspended prior to administration.

In some embodiments, an RSV RNA vaccine composition comprises a preservative (e.g., thiomersal or 2-phenoxy ethanol). In some embodiments, however, an RSV RNA vaccine composition is substantially free from mercurial material e.g., is thiomersal-free. In some embodiments, the RSV RNA vaccine composition is preservative-free.

In some embodiments, an RSV RNA vaccine composition comprises a physiological salt, such as a sodium salt. In some embodiments, an RSV RNA vaccine composition comprises sodium chloride (NaCl). In some embodiments, an RSV RNA vaccine composition comprises NaCl at about 1 to 20 mg/ml. In some embodiments, an RSV RNA vaccine composition comprises NaCl of about 1, 2, 3, 4, 5, 6, 7, 8, 9, or 10 g/L. Other salts that may be present include sodium phosphate, potassium chloride, potassium dihydrogen phosphate, disodium phosphate, disodium phosphate dehydrate, magnesium chloride, magnesium chloride hexahydrate, calcium chloride dihydrate, or other salts known to those of skill in the art. In some embodiments, an RSV RNA vaccine composition comprises monobasic sodium phosphate of about 0.1, 0.2, 0.3, 0.4, or 0.5 g/L. In some embodiments, an RSV RNA vaccine composition comprises dibasic sodium phosphate of about 1, 2, 3, 4, or 5 g/L. In some embodiments, an RSV RNA vaccine composition comprises monobasic sodium phosphate of about 0.1, 0.2, 0.3, 0.4, or 0.5 g/L, and dibasic sodium phosphate of about 1, 2, 3, 4, or 5 g/L. Where adjuvant is in a separate container from antigens, a salt, e.g., sodium chloride may be present in both containers. In some embodiments, an RSV RNA vaccine composition comprises NaCl of about 1, 2, 3, 4, 5, 6, 7, 8, 9, or 10 g/L, monobasic sodium phosphate of about 0.1, 0.2, 0.3, 0.4, or 0.5 g/L, and dibasic sodium phosphate of about 1, 2, 3, 4, or 5 g/L. Where adjuvant is in a separate container from antigens, a salt, e.g., sodium chloride may be present in both containers.

In certain embodiments, acceptable materials included in an RSV RNA vaccine composition are nontoxic to recipients at the dosages and concentrations employed. In certain embodiments, the pharmaceutical composition may contain formulation materials for modifying, maintaining, or preserving, for example, the pH, osmolality, viscosity, clarity, color, isotonicity, odor, sterility, stability, rate of dissolution, or release, adsorption, or penetration of the composition. In such embodiments, suitable formulation materials include, but are not limited to, amino acids (such as glycine, glutamine, asparagine, arginine, or lysine); antimicrobials; antioxidants (such as ascorbic acid, sodium sulfite, or sodium hydrogen-sulfite); buffers (such as borate, bicarbonate, Tris-HCl, citrates, phosphates, or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin, or hydroxypropyl-beta-cyclodextrin); fillers; monosaccharides; disaccharides; and other carbohydrates (such as glucose, sucrose, mannose, or dextrins); proteins (such as serum albumin, gelatin, or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid, or hydrogen peroxide); solvents (such as glycerin, propylene glycol, or polyethylene glycol); sugar alcohols (such as mannitol or sorbitol); suspending agents; surfactants or wetting agents (such as pluronics, PEG, sorbitan esters, polysorbates such as polysorbate-20, polysorbate-80, triton, tromethamine, lecithin, cholesterol, or tyloxapal); stability enhancing agents (such as sucrose or sorbitol); tonicity enhancing agents (such as alkali metal halides, sodium or potassium chloride, or mannitol sorbitol); delivery vehicles; diluents; excipients and/or pharmaceutical adjuvants.

A suitable vehicle or carrier may be water for injection, physiological saline solution, or artificial cerebrospinal fluid. In some embodiments, the vehicle or carrier may be supplemented with other materials common in compositions for parenteral administration. Neutral buffered saline or saline mixed with serum albumin are further exemplary vehicles.

In some embodiments, an RSV RNA vaccine composition comprises one or more buffers. Typical buffers include: a phosphate buffer; a Tris buffer; a borate buffer; a succinate buffer; a histidine buffer (e.g., with an aluminum hydroxide adjuvant); or a citrate buffer. Buffers will typically be included in the 5-20 mM range. The pH of an RSV RNA vaccine composition will generally be 5.0 to 8.1, and more typically 6.0 to 8.0, e.g., 6.5 to 7.5, or 7.0 to 7.8.

In some embodiments, a buffer may be included in an RSV RNA vaccine composition at a concentration of at least 20 mM, at least 30 mM, at least 40 mM, at least 50 mM, at least 60 mM, at least 70 mM, at least 80 mM, at least 90 mM, at least 100 mM, at least 110 mM, at least 120 mM, at least 130 mM, at least 140 mM, or at least 150 mM. In some embodiments, a buffer may be included at a concentration of up to 50 mM, up to 60 mM, up to 70 mM, up to 80 mM, up to 90 mM, up to 100 mM, up to 110 mM, up to 120 mM, up to 130 mM, up to 140 mM, up to 150 mM, or up to 160 mM. In an exemplary embodiment, the buffer includes 30 mM L-histidine/L-histidine hydrochloride.

In some embodiments, an RSV RNA vaccine composition may include a humectant including, for example, sorbitol or a suitable substitute therefor.

RSV RNA vaccine composition components may be present in concentrations that are acceptable to the site of administration. In certain embodiments, a buffer may be used to maintain the composition at physiological pH or at a slightly lower that physiological pH. In some embodiments, the pH of the composition may be at least 5, at least 5.1, at least 5.2, at least 5.3, at least 5.4, at least 5.5, at least 5.6, at least 5.7, at least 5.8, at least 5.9, at least 6.0, at least 6.1, at least 6.2, at least 6.3, at least 6.4, at least 6.5, at least 6.6, at least 6.7, at least 6.8, at least 6.9, at least 7.0, at least 7.1, at least 7.2, at least 7.3, at least 7.4, at least 7.5, at least 7.6, at least 7.7, at least 7.8, or at least 7.9. In some embodiments, the pH of the composition may be up to 5.1, up to 5.2, up to 5.3, up to 5.4, up to 5.5, up to 5.6, up to 5.7, up to 5.8, up to 5.9, up to 6.0, up to 6.1, up to 6.2, up to 6.3, up to 6.4, up to 6.5, up to 6.6, up to 6.7, up to 6.8, up to 6.9, up to 7.0, up to 7.1, up to 7.2, up to 7.3, up to 7.4, up to 7.5, up to 7.6, up to 7.7, up to 7.8, up to 7.9, or up to 8.0. In an exemplary embodiment, the pH of the composition may be in a range of from 5 to 8. In an exemplary embodiment, the pH of the composition may be in a range of from 5.5 to 6.5. In an exemplary embodiment, the pH of the composition may be 6.0.

In some embodiments, an RSV RNA vaccine composition may include an ionic excipient. An ionic excipient may be included in an antibody formulation for the purpose of changing the charge state of the antibody in the formulation, for changing the distribution of the antibody in the formulation, and/or for colloidally stabilizing the antibody in the formulation. In some embodiments, the ionic excipient may include a charged amino acid including, for example, lysine and/or arginine. In some embodiments, the ionic excipient may include a salt including, for example, arginine hydrochloride (arginine-HCl), lysine hydrochloride (lysine-HCl), or sodium chloride (NaCl). In some embodiments, the amino acid or amino acid salt may include the physiological active (e.g., L-form) of the amino acid. In some embodiments, an ionic excipient may be included at a concentration of at least 20 mM, at least 30 mM, at least 40 mM, at least 50 mM, at least 60 mM, at least 70 mM, at least 80 mM, at least 90 mM, at least 100 mM, at least 110 mM, at least 120 mM, at least 130 mM, at least 140 mM, or at least 150 mM. In some embodiments, an ionic excipient may be included at a concentration of up to 50 mM, up to 60 mM, up to 70 mM, up to 80 mM, up to 90 mM, up to 100 mM, up to 110 mM, up to 120 mM, up to 130 mM, up to 140 mM, up to 150 mM, or up to 160 mM. In an exemplary embodiment, an ionic excipient may be present at a concentration in a range of 50 mM to 150 mM. In an exemplary embodiment, an ionic excipient may be present at a concentration in a range of 75 mM to 100 mM. In exemplary embodiments, the ionic excipient may include L-arginine hydrochloride present at a concentration of 75 mM or 80 mM.

In some embodiments, an RSV RNA vaccine composition may further include a sugar including, for example, sucrose. In some embodiments, the composition may include up to 0.5% (w/v) sucrose, up to 1% (w/v) sucrose, up to 5% (w/v) sucrose, up to 10% (w/v) sucrose, or up to 15% (w/v) sucrose. In some embodiments, a sugar may be included at a concentration of at least 50 mM, at least 60 mM, at least 70 mM, at least 80 mM, at least 90 mM, at least 100 mM, at least 110 mM, at least 120 mM, at least 130 mM, at least 140 mM, or at least 150 mM. In some embodiments, a sugar may be included at a concentration of up to 60 mM, up to 70 mM, up to 80 mM, up to 90 mM, up to 100 mM, up to 110 mM, up to 120 mM, up to 130 mM, up to 140 mM, up to 150 mM, or up to 160 mM. In an exemplary embodiment, the sugar includes sucrose at a concentration in a range of 100 mM to 140 mM. For example, the composition may include sucrose at a concentration of 120 mM.

In some embodiments, an RSV RNA vaccine composition may further include a surfactant including, for example, a polysorbate. A polysorbate may include, for example, polysorbate-20, polysorbate-40, polysorbate-60, and polysorbate-80. In some embodiments, the surfactant may be included at a concentration of 0.0001% (w/v), at least 0.001% (w/v), at least 0.002% (w/v), at least 0.01% (w/v), at least 0.02% (w/v), at least 0.03% (w/v), at least 0.04% (w/v), at least 0.05% (w/v), at least 0.06% (w/v), at least 0.07% (w/v), at least 0.08% (w/v), at least 0.09% (w/v), or at least 0.1% (w/v). In some embodiments, the surfactant may be included at a concentration of up to 0.0001% (w/v), up to 0.0005% (w/v), up to 0.001% (w/v), up to 0.002% (w/v), up to 0.01% (w/v), up to 0.02% (w/v), up to 0.03% (w/v), up to 0.04% (w/v), up to 0.05% (w/v), up to 0.06% (w/v), up to 0.07% (w/v), up to 0.08% (w/v), up to 0.09% (w/v), or up to 0.1% (w/v). For example, in an exemplary embodiment, a surfactant may be included in a concentration in a range of 0.001% (w/v) to 0.5% (w/v), in a range of 0.002% (w/v) to 0.1% (w/v), or in a range of 0.01% (w/v) to 0.05% (w/v). In an exemplary embodiment, polysorbate-80 is included in a range of 0.01% (w/v) to 0.05% (w/v). In a further exemplary embodiment, 0.02% (w/v) polysorbate-80 is included in the composition. In another exemplary embodiment, 0.04% (w/v) polysorbate-80 is included in the composition.

An RSV RNA vaccine composition described herein may include detergent, e.g., a polyoxyethylene sorbitan ester surfactant (known as “Tween”), an octoxynol (such as octoxynol-9 (Triton X-100) or t-octylphenoxypolyethoxyethanol), a cetyl trimethyl ammonium bromide (CTAB), or sodium deoxycholate, e.g., for a split or surface antigen vaccine. The detergent may be present only at trace amounts. In some embodiments, an RSV RNA vaccine composition for use in the methods disclosed herein comprises other residual components in trace amounts such as antibiotics (e.g., neomycin, kanamycin, or polymyxin B). Where adjuvant is in a separate container from an RNA encoding an antigen, this detergent will usually be present in the RNA-containing container.

In exemplary embodiments, an RSV RNA vaccine composition is sterile. An RSV RNA vaccine composition is typically non-pyrogenic, e.g., containing less than 0.25 or 0.5 EU (endotoxin unit, a standard measure) per dose. For example, the RSV RNA vaccine composition may contain <0.1 EU per dose. An RSV RNA vaccine composition is typically gluten free.

In some embodiments, an RSV RNA vaccine composition may be stored at −20° C. to −70° C.

In some embodiments, an RSV RNA vaccine composition may be stored at 2° C. to 8° C. In some embodiments, an RSV RNA vaccine composition described herein is stable for extended periods of storage at room temperature or at a temperature in a range of 2° C. to 8° C., including, for example, 5° C. As used herein, room temperature is generally a temperature in the range of 22° C. to 25° C. Suitably an RSV RNA vaccine composition is stable after storage at a temperature in a range of 2° C. to 8° C. (including, for example, 5° C.) for at least one month, at least three months, or at least six months. As used herein, the term “stable” for a period of storage (or “stability”) is used to indicate that the formulations resist aggregation, degradation, half antibody formation, and/or fragmentation.

When parenteral administration is contemplated, an RSV RNA vaccine composition may be provided in the form of a pyrogen-free, parenterally acceptable aqueous solution including an RSV RNA vaccine in a pharmaceutically acceptable vehicle. In some embodiments, a suitable vehicle for parenteral injection is sterile distilled water in which the antibody is formulated as a sterile, isotonic solution, properly preserved. Additionally or alternatively, formulations suitable for parenteral administration may include a sterile aqueous preparation of an RSV RNA vaccine composition, or dispersions of sterile powders of an RSV RNA vaccine composition, which may be isotonic with the blood of the recipient. Isotonic agents that can be included in the liquid preparation include sugars, buffers, and sodium chloride. Solutions of the anti-RSV antibody or an antigen binding fragment thereof can be prepared in water. In certain embodiments, solutions of the anti-RSV antibody or an antigen binding fragment thereof prepared in water can be mixed with a nontoxic surfactant.

Dispersions of an RSV RNA vaccine can be prepared in water, ethanol, a polyol (such as glycerol, propylene glycol, liquid polyethylene glycols, and the like), vegetable oils, glycerol esters, and mixtures thereof. The ultimate dosage form may, in some embodiments, be sterile, fluid, and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the anti-RSV antibody or an antigen binding fragment thereof, for example, by filter sterilization. Methods for preparing powders include vacuum drying and freeze drying of the sterile injectable solutions. Subsequent microbial contamination can be prevented using various antimicrobial agents, for example, antibacterial, antiviral, and antifungal agents including parabens, chlorobutanol, phenol, sorbic acid, thimerosal, and the like. Absorption of the anti-RSV antibody or an antigen binding fragment thereof over a prolonged period can be achieved by including agents for delaying absorption, for example, aluminum monostearate and gelatin.

In exemplary embodiments, an RSV RNA vaccine composition is provided as a liquid solution in a vial. The vaccine may be kept frozen until use. In some embodiments, an RSV RNA vaccine is provided as a 0.5 mL dose that contains 10 micrograms, 30 micrograms, 75 micrograms, or 110 micrograms of mRNA. In certain embodiments, an RSV RNA vaccine is diluted with 2.2×PBS (2° C. to 8° C.).

### Adjuvants

In some embodiments, an RSV RNA vaccine composition does not comprise an adjuvant. In some embodiments, an RSV RNA vaccine composition comprises one or more adjuvants, which can function to enhance the immune responses (humoral and/or cellular) elicited in a subject who receives the composition. In some embodiments, an RSV RNA vaccine composition comprises an oil-in-water emulsion adjuvant. In some embodiments, an RSV RNA vaccine composition comprises squalene.

In some embodiments, an RSV RNA vaccine composition comprises oil-in-water emulsions and at least one surfactant.

In some embodiments, an RSV RNA vaccine composition comprises one or more tocopherols.

In some embodiments, an RSV RNA vaccine composition comprises tocopherols and squalene. In some embodiments, the oil content is in the range of 2-20% (by volume).

In some embodiments, an RSV RNA vaccine composition comprises an adjuvant comprising a mineral-containing composition, including calcium salts and aluminum salts (or mixtures thereof). Calcium salts include calcium phosphate. Aluminum salts include hydroxides, phosphates, sulfates, etc., with the salts taking any suitable form (e.g., gel, crystalline, amorphous, etc.). The mineral-containing compositions may also be formulated as a particle of metal salt.

In some embodiments, an RSV RNA vaccine composition comprises an adjuvant comprising one or more saponins, which are a heterologous group of sterol glycosides and triterpenoid glycosides that are found in the bark, leaves, stems, roots, and flowers of a wide range of plant species. Saponin from the bark of the Quillaja saponaria Molina tree have been widely studied as adjuvants. Saponin can also be commercially obtained from Smilax ornata (sarsaparilla), Gypsophilla paniculate (brides veil), and Saponaria officianalis.

Saponin adjuvant formulations include purified formulations, such as QS21, as well as lipid formulations, such as ISCOMs. QS21 is marketed as STIMULON®. Combinations of saponins and cholesterols can be used to form unique particles called immunostimulating complexes (ISCOMs). In some embodiments, an ISCOM includes a phospholipid such as phosphatidylethanolamine or phosphatidylcholine. Any known saponin can be used in ISCOMs. In exemplary embodiments, the ISCOM includes one or more of: QuilA, QHA, and QHC.

In some embodiments, an RSV RNA vaccine composition comprises an adjuvant comprising fatty adjuvants.

In some embodiments, an RSV RNA vaccine composition comprises an adjuvant comprising bacterial ADP-ribosylating toxins (e.g., the E. coli heat labile enterotoxin “LT,” cholera toxin “CT,” or pertussis toxin “PT”) and detoxified derivatives thereof, such as the mutant toxins known as LT-K63 and LT-R72.

In some embodiments, an RSV RNA vaccine composition comprises an adjuvant comprising bioadhesives and mucoadhesives, such as esterified hyaluronic acid microspheres or chitosan and its derivatives.

In some embodiments, an RSV RNA vaccine composition comprises an adjuvant comprising cytokine-inducing agents.

In some embodiments, an RSV RNA vaccine composition comprises an adjuvant comprising liposomes.

In some embodiments, an RSV RNA vaccine composition comprises an adjuvant comprising polyoxyethylene ethers and/or polyoxyethylene esters. Such formulations further include polyoxyethylene sorbitan ester surfactants in combination with an octoxynol as well as polyoxyethylene alkyl ethers or ester surfactants in combination with at least one additional non-ionic surfactant such as an octoxynol. Exemplary polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

In some embodiments, an RSV RNA vaccine composition comprises an adjuvant comprising muramyl peptides, such as N-acetylmuramyl-L-threonyl-D-isoglutamine (thr-MDP), N-acetyl-normuramyl-L-alanyl-D-isoglutamine (nor-MDP), N-acetylglucsaminyl-N-acetylmuramyl-L-AI-D-isoglu-L-Ala-dipalmitoxy propylamide (DTP-DPP or THERAMIDE™), N-acetylrnuramyl-L-alanyl-D-isoglutaminyl-L-alanine-2-(1′-2′dipalmitoyl-sn-glycero-3-hydroxyphosphoryloxy)-ethylamine (MTP-PE).

An RSV RNA vaccine composition may include one or more adjuvants, e.g., 2, 3, 4, or more adjuvants. For example, an RSV RNA vaccine composition may include both an oil-in-water emulsion and a cytokine-inducing agent.

### IV. Methods of Vaccination

The RSV vaccine disclosed herein may be administered to a subject to induce an immune response directed against the RSV F protein, wherein an anti-antigen antibody titer in the subject is increased following vaccination relative to an anti-antigen antibody titer in a subject that is not vaccinated with the RSV vaccine disclosed herein, or relative to an alternative vaccine against RSV. An “anti-antigen antibody” is a serum antibody that binds specifically to the antigen.

In one aspect, the disclosure provides a method of eliciting an immune response to RSV or protecting a subject against RSV infection comprising administering the RSV vaccine described herein to a subject. The disclosure also provides an RSV vaccine described herein for use in eliciting an immune response to RSV or in protecting a subject against RSV infection. The disclosure also provides an RSV mRNA described herein for use in the manufacture of a vaccine for eliciting an immune response to RSV or for protecting a subject against RSV infection.

In certain embodiments, the subject has a higher serum concentration of neutralizing antibodies against RSV after administration of the RSV vaccine, relative to a subject that is administered an RSV vaccine comprising an mRNA ORF encoding an RSV F protein antigen of SEQ ID NO: 1.

In certain embodiments, the subject has a comparable serum concentration of neutralizing antibodies against RSV after administration of the RSV vaccine, relative to a subject that is administered an RSV protein vaccine that is co-administered with an adjuvant.

In certain embodiments, the RSV vaccine increases the serum concentration of antibodies with binding specificity to site Ø of the RSV F protein.

In certain embodiments, the subject has a lower serum concentration of antibodies with binding specificity to site I or site II of the RSV F protein after administration of the RSV vaccine, relative to a subject that is administered an RSV vaccine comprising an mRNA ORF encoding an RSV F protein antigen of SEQ ID NO: 2.

In certain embodiments, the RSV vaccine increases the serum concentration of neutralizing antibodies in a subject with pre-existing RSV immunity.

### V. RSV F Proteins

In one aspect, the disclosure provides a respiratory syncytial virus (RSV) vaccine comprising a messenger RNA (mRNA) comprising an open reading frame (ORF) encoding an RSV F protein antigen.

In some embodiments, the ORF is codon optimized. Codon optimization can refer to the introduction of certain codons (in exchange for the respective wild-type codons encoding the same amino acid), which may be more favorable with respect to stability of RNA and/or with respect to codon usage in a subject.

In some embodiments, an epitope of the RSV F protein that is shared between Pre-F and Post-F is blocked. Blocking an epitope reduces or eliminates the generation of antibodies against the epitope when the RNA (e.g., mRNA) that encodes for the antigenic RSV F polypeptide is administered to a subject. This can increase the proportion of antibodies that target an epitope specific to a particular conformation of F, such as the pre-fusion conformation (e.g., antibodies that target site Ø). Because F has the pre-fusion conformation in viruses that have not yet entered cells, an increased proportion of antibodies that target Pre-F can provide a greater degree of neutralization (e.g., expressed as a neutralizing to binding ratio, as described herein). Blocking can be achieved by engineering a bulky moiety such as an N-glycan in the vicinity of the shared epitope. For example, an N-glycosylation site not present in wild-type F can be added, e.g., by mutating an appropriate residue to asparagine. In some embodiments, the blocked epitope is an epitope of antigenic site I of RSV F. In some embodiments, two or more epitopes shared between pre-F and post-F are blocked. In some embodiments, two or more epitopes of antigenic site I of RSV F are blocked. In some embodiments, one or more, or all, epitopes that topologically overlap with the blocked epitope are also blocked. The blocked epitope may be an epitope of antigenic site I of RSV F.

In some embodiments, the RSV F polypeptide comprises an asparagine substitution at one or more positions corresponding to position 328, 348, or 507 of SEQ ID NO: 1 (i.e., E328N, S348N, or R507N). In some embodiments, the RSV F polypeptide comprises an asparagine substitution at two or more positions corresponding to position 328, 348, or 507 of SEQ ID NO: 1 (i.e., E328N, S348N, or R507N). In some embodiments, the RSV F polypeptide comprises an asparagine substitution at positions 328, 348, and 507 of SEQ ID NO: 1 (i.e., E328N, S348N, and R507N).

Asparagines can function as glycosylation sites (see WO2019/195291, incorporated herein by reference). Furthermore, without wishing to be bound by any particular theory, glycans at these sites may inhibit development of antibodies to nearby epitopes, which include epitopes common to pre- and post-fusion RSV F protein, when the RNA (e.g., mRNA) that encodes for the antigenic RSV F polypeptide is administered to a subject. In some embodiments, glycosylation of the asparagine corresponding to position 328, 348, or 507 of SEQ ID NO: 1 blocks at least one epitope shared between pre-fusion RSV F and post-fusion RSV F, such as an epitope of antigenic site 1. Inhibiting the development of antibodies to epitopes common to pre- and post-fusion RSV F protein can be beneficial because it can direct antibody development against epitopes specific to pre-fusion RSV F protein, such as the site Ø epitope, which may have more effective neutralizing activity than antibodies to other RSV F epitopes. The site Ø epitope involves amino acid residues 62-69 and 196-209 of SEQ ID NO: 1. Accordingly, in some embodiments, the RSV F polypeptide comprises amino acid residues 62-69 and 196-209 of SEQ ID NO: 1.

The RSV F polypeptides described herein may have deletions or substitutions of different length relative to wild type RSV F. For example, in the RSV F polypeptide of SEQ ID NO: 1, positions 98-144 of the wild-type sequence (SEQ ID NO: 1) are replaced with GSGNVGL (SEQ ID NO: 15), resulting in a net removal of 40 amino acids, such that positions 328, 348, or 507 of SEQ ID NO: 1 correspond to positions 288, 308, and 467 of SEQ ID NO: 3. In the alternative, in the RSV F polypeptide of SEQ ID NO: 3, positions 98-146 of the wild-type sequence (SEQ ID NO: 1) are replaced with GSGNVGLGG (SEQ ID NO: 16, positions 98-106 of SEQ ID NO: 3), resulting in a net removal of 40 amino acids, such that positions 328, 348, or 507 of SEQ ID NO: 1 correspond to positions 290, 310, and 469 of SEQ ID NO: 3.

In general, positions in constructs described herein can be mapped onto the wild-type sequence of SEQ ID NO: 1 by pairwise alignment, e.g., using the Needleman-Wunsch algorithm with standard parameters (EBLOSUM62 matrix, Gap penalty 10, gap extension penalty 0.5). See also the discussion of structural alignment provided herein as an alternative approach for identifying corresponding positions.

In some embodiments, the RSV F polypeptide comprises mutations that add glycans to block epitopes on the pre-fusion antigen that are structurally similar to those on the surface of the post-fusion RSV F. In some embodiments, glycans are added to specifically block epitopes that may be present in the post-fusion conformation of RSV F. In some embodiments, glycans are added that block epitopes that may be present in the post-fusion conformation of RSV F but do not affect one or more epitopes present on the pre-fusion conformation of RSV F, such as the site Ø epitope.

In some embodiments, the RSV F polypeptide comprises a sequence having at least 85%, 90%, 95%, 97%, 98%, 99%, or 99.5% identity to an amino acid sequence set forth in SEQ ID NO: 1.

In some embodiments, the RSV F polypeptide comprises a sequence having at least 85%, 90%, 95%, 97%, 98%, 99%, or 99.5% identity to an amino acid sequence set forth in SEQ ID NO: 2.

In some embodiments, the RSV F polypeptide comprises a sequence having at least 85%, 90%, 95%, 97%, 98%, 99%, or 99.5% identity to an amino acid sequence set forth in SEQ ID NO: 3.

In some embodiments, the RSV F polypeptide comprises the DS-CAV1 amino acid substitutions (as described, for example, in McLellan et al., Science, 342(6158): 592-598, 2013) in which further modifications are made including at least one, two, or three of the asparagines described above. The CAV1 mutations are S190F and V207L relative to SEQ ID NO: 1. The DS mutations are S155C and S290C relative to SEQ ID NO: 1.

In some embodiments, an amino acid substitution or pair of amino acid substitutions are inter-protomer stabilizing substitution(s). Exemplary substitutions that can be inter-protomer stabilizing are V207L; N228F; I217V and E218F; 1221L and E222M; or Q224A and Q225L, using the position numbering of SEQ ID NO: 1.

In some embodiments, an amino acid substitution or pair of amino acid substitutions are intra-protomer stabilizing. Exemplary substitutions that can be intra-protomer stabilizing are V2201; and A74L and Q81 L, using the position numbering of SEQ ID NO: 1.

In some embodiments, an amino acid substitution is helix stabilizing, i.e., predicted to stabilize the helical domain of RSV F. Stabilization of the helical domain can contribute to the stability of the site Ø epitope and of the pre-fusion conformation of RSV F generally. Exemplary substitutions that can be helix stabilizing are N216P or I217P, using the position numbering of SEQ ID NO: 1. Position 217 in SEQ ID NO: 1 corresponds to position 177 in SEQ ID NO: 3.

In some embodiments, an amino acid substitution is helix capping. In some embodiments, an amino acid substitution is helix PRO capping. Helix capping is based on the biophysical observation that, while a proline residue mutation placed in an alpha helix may disrupt the helix formation, a proline at the N-terminus of a helical region may help induce helical formation by stabilizing the PHI/PSI bond angles. Exemplary substitutions that can be helix capping are N216P or I217P, using the position numbering of SEQ ID NO: 1.

In some embodiments, an amino acid substitution replaces a disulfide mutation of DS-CAV1. In some embodiments, the engineered disulfide of DS-CAV1 is reverted to wild-type (C69S and/or C212S mutations of DS-CAV1 using the position numbering of SEQ ID NO: 1). In some embodiments, one or more C residue of DS-CAV1 is replaced with a S residue to eliminate a disulfide bond. In some embodiments, C69S or C212S substitution using the position numbering of SEQ ID NO: 1 eliminates a disulfide bond. In some embodiments, an RSV F polypeptide comprises both C69S and C212S using the position numbering of SEQ ID NO: 1. In some embodiments, replacing such cysteines and thereby eliminating a disulfide bond blocks reduction (i.e., acceptance of electrons from a reducing agent) of the RSV F polypeptide. In some embodiments, an I217P substitution using the position numbering of SEQ ID NO: 1 is comprised in an antigen instead of substitution at C69 and/or C212.

In some embodiments, an amino acid substitution prevents proteolysis by trypsin or trypsin-like proteases. In some embodiments, the amino acid substitution that prevents such proteolysis is in the heptad repeat region B (HRB) region of RSV F.

Appearance of fragments consistent with proteolysis of an RSV F polypeptide that comprised a wild-type HRB region suggested a lysine or arginine in this region was being targeted for proteolysis. An amino acid substitution to remove a K or R residue may be termed a knockout (KO). In some embodiments, a K or R is substituted for L or Q. In some embodiments, a K is substituted for L or Q. In some embodiments, the RSV F polypeptide comprises K498L and/or K508Q, using the position numbering of SEQ ID NO: 1. The corresponding positions in SEQ ID NO: 3 are 458 and 468, respectively. In some embodiments, the RSV F polypeptide comprises both K498L and K508Q.

In some embodiments, an amino acid substitution adds glycans. In some embodiments, an amino acid substitution increases glycosylation by adding glycans to RSV F polypeptides. Substitutions to add glycans may also be referred to as engineered glycosylation, as compared to native glycosylation (without additional glycans).

In some embodiments, the amino acid substitution to add glycans is substitution with an N. In some embodiments, amino acid substitution with an N allows N-linked glycosylation. In some embodiments, substitution with an N is accompanied by substitution with a T or S at the second amino acid position C-terminal to the N, which forms an NxT/S glycosylation motif. In some embodiments, the N is surface-exposed.

Each of the above recited substitutions and mutations in the RSV F polypeptide are described in more detail in WO2019/195291, which is incorporated herein by reference.

In one aspect, the disclosure provides, a respiratory syncytial virus (RSV) vaccine, comprising a messenger RNA (mRNA) comprising an open reading frame (ORF) encoding an RSV F protein antigen, wherein the RSV F protein antigen comprises one or more of the following substitutions relative to an amino acid sequence set forth in SEQ ID NO: 1:
1) amino acid positions 98-146 of SEQ ID NO: 1 are replaced with an amino acid sequence of GSGNVGLGG (SEQ ID NO: 16);
2) amino acid substitutions S190F and V207L;
3) amino acid substitution I217P;
4) amino acid substitutions E328N, S348N, and R507N;
5) amino acid substitution L373R;
6) amino acid substitution K498L; and
7) amino acid substitution K508Q.

In another aspect, the disclosure provides an RSV vaccine comprising an mRNA comprising an ORF encoding an RSV F protein antigen, wherein the RSV F protein antigen comprises each of the following substitutions relative to an amino acid sequence set forth in SEQ ID NO: 1:
1) amino acid positions 98-146 of SEQ ID NO: 1 are replaced with an amino acid sequence of GSGNVGLGG (SEQ ID NO: 16);
2) amino acid substitutions S190F and V207L;
3) amino acid substitution I217P;
4) amino acid substitutions E328N, S348N, and R507N;
5) amino acid substitution L373R;
6) amino acid substitution K498L; and
7) amino acid substitution K508Q.

In certain embodiments, the RSV F protein antigen comprises a transmembrane domain and cytoplasmic tail amino acid sequence of IMITTIIIVIIVILLSLIAVGLLLYCKARSTPVTLSKDQLSGINNIAFSN (SEQ ID NO: 17).

In some embodiments, the mRNA comprises a nucleic acid sequence with at least 80%, at least 85%, at least 90%, at least 91%, at least 92%, at least 93%, at least 94%, at least 95%, at least 96%, at least 97%, at least 98%, at least 99%, or 100% identity to a nucleic acid sequence set forth in any one of SEQ ID NOs: 4-6.In some embodiments, the mRNA comprises a nucleic acid sequence with at least 80%, at least 85%, at least 90%, at least 91%, at least 92%, at least 93%, at least 94%, at least 95%, at least 96%, at least 97%, at least 98%, at least 99%, or 100% identity to a nucleic acid sequence set forth in any one of SEQ ID NOs: 12-14.

### VI. RSV RNA

The RSV vaccines of the present disclosure may comprise at least one ribonucleic acid (RNA) comprising an ORF encoding an RSV F protein antigen.

In certain embodiments, the RNA is an mRNA comprising an ORF encoding an RSV F protein antigen. In certain embodiments, the RNA (e.g., mRNA) further comprises at least one 5′ UTR, 3′ UTR, a poly(A) tail, and/or a 5′ cap.

In some embodiments, an RSV mRNA has at least 85%, 90%, 95%, 97%, 98%, 99%, 99.5%, or 100% sequence identity to a nucleic acid sequence set forth as SEQ ID NO: 12.

In some embodiments, an RSV mRNA has at least 85%, 90%, 95%, 97%, 98%, 99%, 99.5%, or 100% sequence identity to a nucleic acid sequence set forth as SEQ ID NO: 13.

In some embodiments, an RSV mRNA has at least 85%, 90%, 95%, 97%, 98%, 99%, 99.5%, or 100% sequence identity to a nucleic acid sequence set forth as SEQ ID NO: 14.

### II. A. 5′ Cap

An mRNA 5′ cap can provide resistance to nucleases found in most eukaryotic cells and promote translation efficiency. Several types of 5′ caps are known. A 7-methylguanosine cap (also referred to as “m⁷G” or “Cap-O”) comprises a guanosine that is linked through a 5′-5′-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5 ′5 ′5 triphosphate linkage; and the 7-nitrogen of guanine is then methylated by a methyltransferase. Examples of cap structures include, but are not limited to, m7G(5′)ppp, (5′(A,G(5′)ppp(5′)A, and G(5′)ppp(5′)G. Additional cap structures are described in U.S. Publication No. US 2016/0032356 and U.S. Publication No. US 2018/0125989, which are incorporated herein by reference.

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′OMeA)pG; m7G(5′)ppp(5′)(2′OMeA)pU; m7G(5′)ppp(5′)(2′OMeG)pG (New England BioLabs, Ipswich, MA; TriLink Biotechnologies). 5′-capping of modified RNA may be completed post-transcriptionally using a vaccinia virus capping enzyme to generate the Cap 0 structure: m7G(5′)ppp(5′)G. Cap 1 structure may be generated using both vaccinia virus capping enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase.

In certain embodiments, the mRNA of the disclosure comprises a 5′ cap selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

In certain embodiments, the mRNA of the disclosure comprises a 5′ cap of:

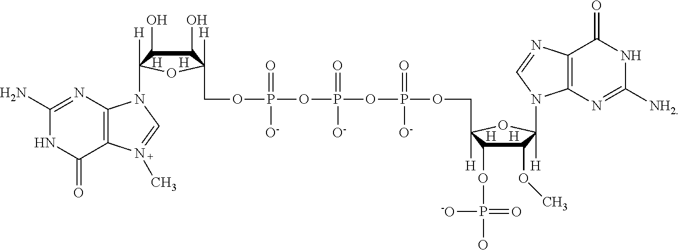

### II. B. Untranslated Region (UTR)

In some embodiments, the mRNA of the disclosure includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein may comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is at least about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence derived from a CMV immediate-early 1 (IE1) gene (U.S. Publication Nos. 2014/0206753 and 2015/0157565, each of which is incorporated herein by reference), or the sequence GGGAUCCUACC (SEQ ID NO: 18) (U.S. Publication No. 2016/0151409, incorporated herein by reference).

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of an hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

In some embodiments, the 5′ UTR comprises a nucleic acid sequence set forth in SEQ ID NO: 10. In some embodiments, the 3′ UTR comprises a nucleic acid sequence set forth in SEQ ID NO: 11. The 5′ UTR and 3′ UTR are described in further detail in WO2012/075040, incorporated herein by reference.

### II. C. Polyadenylated Tail

As used herein, the terms “poly(A) sequence,” “poly(A) tail,” and “poly(A) region” refer to a sequence of adenosine nucleotides at the 3′ end of the mRNA molecule. The poly(A) tail may confer stability to the mRNA and protect it from exonuclease degradation. The poly(A) tail may enhance translation. In some embodiments, the poly(A) tail is essentially homopolymeric. For example, a poly(A) tail of 100 adenosine nucleotides may have essentially a length of 100 nucleotides(SEQ ID NO: 20). In certain embodiments, the poly(A) tail may be interrupted by at least one nucleotide different from an adenosine nucleotide (e.g., a nucleotide that is not an adenosine nucleotide). For example, a poly(A) tail of 100 adenosine nucleotides may have a length of more than 100 nucleotides (comprising 100 adenosine nucleotides and at least one nucleotide, or a stretch of nucleotides, that are different from an adenosine nucleotide). In certain embodiments, the poly(A) tail comprises the sequence AAAAAAAAAAAAAAAAAAAAAAAAAAAAAAGCAUAUGACUAAAAAAAAAAAAAAAAAAAA AAAAAAAAAAAAAAAAAAAAAAAAAAAAAAAAAAAAAAAAAAAAAAAAAA (SEQ ID NO: 19).

The “poly(A) tail,” as used herein, typically relates to RNA. However, in the context of the disclosure, the term likewise relates to corresponding sequences in a DNA molecule (e.g., a “poly(T) sequence”).

The poly(A) tail may comprise about 10 to about 500 adenosine nucleotides, about 10 to about 200 adenosine nucleotides, about 40 to about 200 adenosine nucleotides, or about 40 to about 150 adenosine nucleotides(SEQ ID NO: 20). The length of the poly(A) tail may be at least about 10, 50, 75, 100, 150, 200, 250, 300, 350, 400, 450, or 500 (SEQ ID NO: 20) adenosine nucleotides.

In some embodiments where the nucleic acid is an RNA, the poly(A) tail of the nucleic acid is obtained from a DNA template during RNA in vitro transcription. In certain embodiments, the poly(A) tail is obtained in vitro by common methods of chemical synthesis without being transcribed from a DNA template. In various embodiments, poly(A) tails are generated by enzymatic polyadenylation of the RNA (after RNA in vitro transcription) using commercially available polyadenylation kits and corresponding protocols, or alternatively, by using immobilized poly(A)polymerases, e.g., using methods and means as described in WO2016/174271.

The nucleic acid may comprise a poly(A) tail obtained by enzymatic polyadenylation, wherein the majority of nucleic acid molecules comprise about 100 (+/−20) to about 500 (+/−50) or about 250 (+/−20) adenosine nucleotides.

In some embodiments, the nucleic acid may comprise a poly(A) tail derived from a template DNA and may additionally comprise at least one additional poly(A) tail generated by enzymatic polyadenylation, e.g., as described in WO2016/091391. In certain embodiments, the nucleic acid comprises at least one polyadenylation signal. In various embodiments, the nucleic acid may comprise at least one poly(C) sequence. The term “poly(C) sequence,” as used herein, is intended to be a sequence of cytosine nucleotides of up to about 200 cytosine nucleotides. In some embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides(SEQ ID NO: 21). In some embodiments, the poly(C) sequence comprises about 30 cytosine nucleotides(SEQ ID NO: 21).

### II. D. Chemical Modification

The mRNA disclosed herein may be modified or unmodified. In some embodiments, the mRNA may comprise at least one chemical modification. In some embodiments, the mRNA disclosed herein may contain one or more modifications that typically enhance RNA stability. Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

In some embodiments, the disclosed mRNA may comprise at least one chemical modification including, but not limited to, pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is described, e.g., in U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

II. E. mRNA Synthesis

The mRNAs disclosed herein may be synthesized according to any of a variety of methods. For example, mRNAs according to the present disclosure may be synthesized via in vitro transcription (IVT). Some methods for in vitro transcription are described, e.g., in Geall et al. (2013) Semin. Immunol. 25(2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, an appropriate RNA polymerase (e.g., T3, T7, or SP6 RNA polymerase), DNase I, pyrophosphatase, and/or RNase inhibitor. The exact conditions may vary according to the specific application. The presence of these reagents is generally undesirable in a final mRNA product and these reagents can be considered impurities or contaminants which can be purified or removed to provide a clean and/or homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

In certain embodiment, the mRNA comprises of the following structural elements:
(i) a 5′ cap with the following structure:

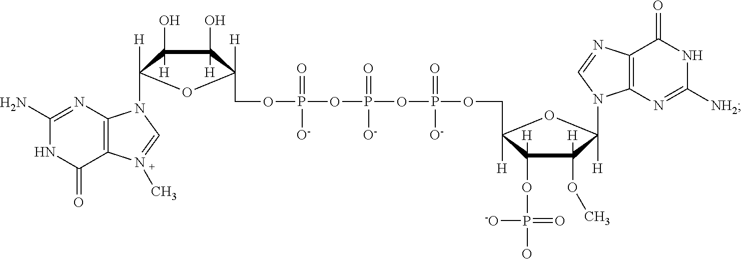

(ii) a 5′ untranslated region (5′ UTR) having the nucleic acid sequence of SEQ ID NO: 10;
(iii) a protein coding region having the nucleic acid sequence of SEQ ID NO: 6;
(iv) a 3′ untranslated region (3′ UTR) having the nucleic acid sequence of SEQ ID NO: 11; and
(v) a poly(A) tail.

In certain embodiments, the poly(A) tail has a length of about 10 to about 500 adenosine nucleotides(SEQ ID NO: 20).

### VII. Lipid Nanoparticle (LNP)

The LNPs of the disclosure can comprise four categories of lipids: (i) an ionizable lipid (e.g., cationic lipid); (ii) a PEGylated lipid; (iii) a cholesterol-based lipid (e.g., cholesterol), and (iv) a helper lipid.

### A. Cationic Lipid

An ionizable lipid facilitates mRNA encapsulation and may be a cationic lipid. A cationic lipid affords a positively charged environment at low pH to facilitate efficient encapsulation of the negatively charged mRNA drug substance. Exemplary cationic lipids are shown below in Table 1.

TABLE 1
Ionizable Lipids

| Name | Structure |
|---|---|
| OF-02 (ML7) | 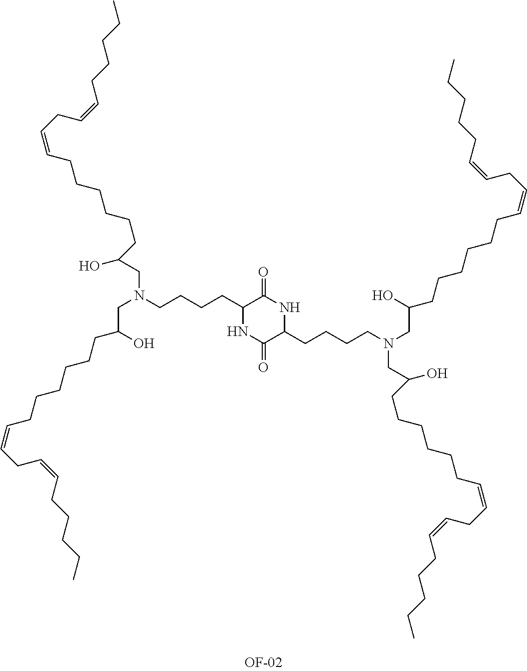 [SMILES: CCCCC/C=C\C/C=C\CCCCCCC(O)CN(CCCCC1NC(=O)C(CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC] |
| cKK-E10 | 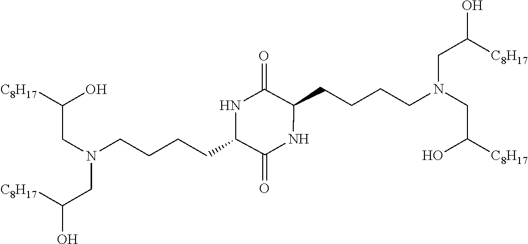 |
| GL-HEPES-E3- E10-DS-3-E18- 1 ((2-(4-(2-((3- (Bis((Z)-2- hydroxyoctadec- 9-en-1- yl)amino)propyl) disulfaneyl)ethyl) piperazin-1- yl)ethyl 4-(bis(2- hydroxydecyl)a mino)butanoate) | 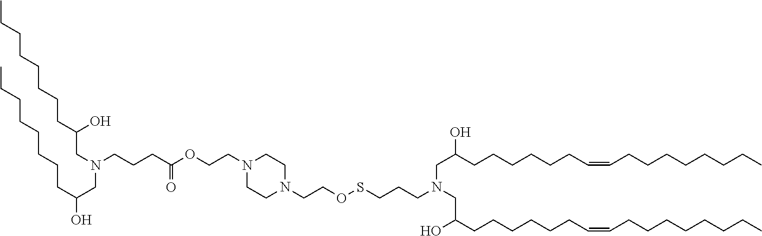 [SMILES: CCCCCCCC/C=C\CCCCCCC(O)CN(CCCSOCCN1CCN(CCOC(=O)CCCN(CC(O)CCCCCCCC)CC(O)CCCCCCCC)CC1)CC(O)CCCCCC/C=C\CCCCCCCC] |
| GL-HEPES-E3- E12-DS-4-E10 (2-(4-(2-((3- (bis(2- hydroxydecyl) amino)butyl) disulfaneyl)ethyl) piperazin-1-yl) ethyl 4-(bis(2- hydroxydodecyl) amino)butanoate) | 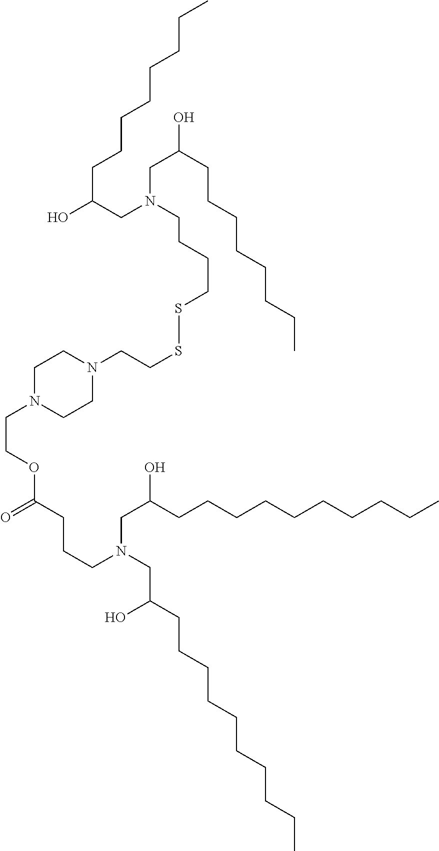 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC] |
| GL-HEPES-E3- E12-DS-3-E14 (2-(4-(2-((3- (Bis(2- hydroxytetradec yl)amino)propyl) disulfaneyl)ethyl) piperazin-1- yl)ethyl 4-(bis(2- hydroxydodecyl) amino)butanoate) | 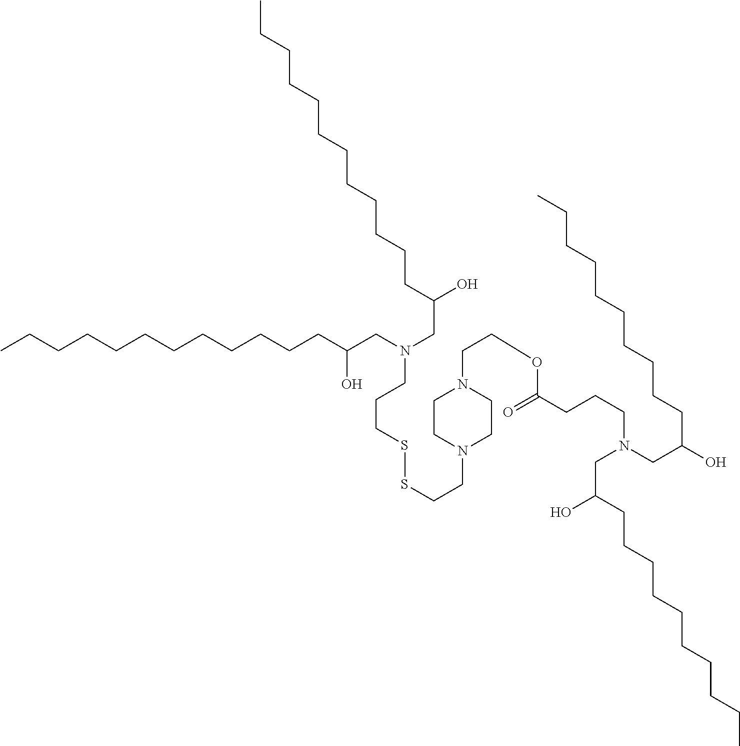 [SMILES: CCCCCCCCCCCCC(O)CN(CCCSSCCN1CCN(CCOC(=O)CCCN(CC(O)CCCCCCCCCC)CC(O)CCCCCCCCCC)CC1)CC(O)CCCCCCCCCCCC] |
| MC3 | 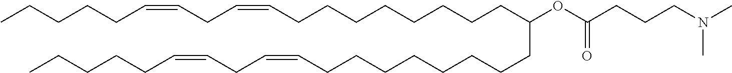 [SMILES: CCCCC/C=C\C/C=C\CCCCCCCCC(CCCCCCCC/C=C\C/C=C\CCCCC)OC(=O)CCCN(C)C] |
| SM-102 (9-heptadecanyl 8-{(2- hydroxyethyl)[6- oxo-6- (undecyloxy) hexyl]amino} octanoate) | 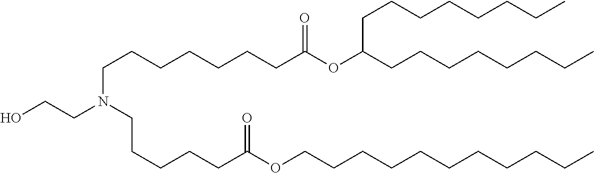 [SMILES: CCCCCCCCCCCOC(=O)CCCCCN(CCO)CCCCCCCC(=O)OC(CCCCCCCC)CCCCCCCC] |
| ALC-0315 [(4- hydroxybutyl) azanediyl]di (hexane-6,1-diyl) bis(2- hexyldecanoate) | 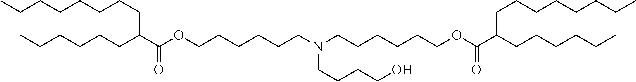 [SMILES: CCCCCCCCC(CCCCCC)C(=O)OCCCCCCN(CCCCO)CCCCCCOC(=O)C(CCCCCC)CCCCCCCC] |
| IM-001 | 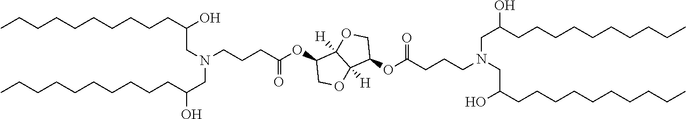 [SMILES: CCCCCCCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC] |

The cationic lipid may be selected from the group comprising [ckkE10]/[OF-02], [(6Z,9Z,28Z,31Z)-heptatriaconta-6,9,28,31-tetraen-19-yl]4-(dimethylamino)butanoate (D-Lin-MC3-DMA); 2,2-dilinoleyl-4-dimethylaminoethyl-[1,3]-dioxolane (DLin-KC2-DMA); 1,2-dilinoleyloxy-N,N-dimethyl-3-aminopropane (DLin-DMA); di((Z)-non-2-en-1-yl) 9-((4-(dimethylamino)butanoyl)oxy)heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl]di(hexane-6,1-diyl) bis(2-hexyldecanoate) (ALC-0315); [3-(dimethylamino)-2-[(Z)-octadec-9-enoyl]oxypropyl](Z)-octadec-9-enoate (DODAP); 2,5-bis(3-aminopropylamino)-N-[2-[di(heptadecyl)amino]-2-oxoethyl]pentanamide (DOGS); [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl]N-[2-(dimethylamino)ethyl]carbamate (DC-Chol); tetrakis(8-methylnonyl) 3,3′,3″,3′″-(((methylazanediyl) bis(propane-3,1 diyl))bis (azanetriyl))tetrapropionate (306Oi10); decyl (2-(dioctylammonio)ethyl) phosphate (9A1P9); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-016B); 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol) (C12-200); 3,6-bis(4-(bis(2-hydroxydodecyl)amino)butyl)piperazine-2,5-dione (cKK-E12); hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate (FTT5); (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate) (OF-Deg-Lin); TT3; N¹,N³,N⁵-tris(3-(didodecylamino)propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl)(8-(nonyloxy)-8-oxooctyl)amino)octanoate (Lipid 5); IM-001; and combinations thereof.

In certain embodiments, the cationic lipid is biodegradable.

In various embodiments, the cationic lipid is not biodegradable.

In some embodiments, the cationic lipid is cleavable.

In certain embodiments, the cationic lipid is not cleavable.

Cationic lipids are described in further detail in Dong et al. (PNAS. 111(11):3955-60. 2014); Fenton et al. (Adv Mater. 28:2939. 2016); U.S. Pat. Nos. 9,512,073; and 10,201,618, each of which is incorporated herein by reference.

### B. PEGylated Lipid

The PEGylated lipid component can provide control over particle size and stability of the nanoparticle. The addition of such components may prevent complex aggregation and provide a means for increasing circulation lifetime and increasing the delivery of the lipid-nucleic acid pharmaceutical composition to target tissues (Klibanov et al. FEBS Letters 268(1):235-7. 1990). These components may be selected to rapidly exchange out of the pharmaceutical composition in vivo (see, e.g., U.S. Pat. No. 5,885,613).

Contemplated PEGylated lipids include, but are not limited to, a polyethylene glycol (PEG) chain of up to 5 kDa in length covalently attached to a lipid with alkyl chain(s) of C₆-C₂₀ (e.g., C₈, C₁₀, C₁₂, C₁₄, C₁₆, or C₁₈) length, such as a derivatized ceramide (e.g., N-octanoyl-sphingosine-1-[succinyl(methoxypolyethylene glycol)](C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1,2-distearoyl-rac-glycero-polyethelene glycol (DSG-PEG), PEG-DAG; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

In certain embodiments, the PEG has a high molecular weight, e.g., 2000-2400 g/mol. In certain embodiments, the PEG is PEG2000 (or PEG-2K). In certain embodiments, the PEGylated lipid herein is DMG-PEG2000, DSPE-PEG2000, DLPE-PEG2000, DSG-PEG2000, C8 PEG2000, or ALC-0159 (2-[(polyethylene glycol)-2000]-N,N-ditetradecylacetamide). In certain embodiments, the PEGylated lipid herein is DMG-PEG2000.

### C. Cholesterol-Based Lipid

The cholesterol component can provide stability to the lipid bilayer structure within the nanoparticle. In some embodiments, the LNPs comprise one or more cholesterol-based lipids. Suitable cholesterol-based lipids include, for example: DC-Choi (N,N-dimethyl-N-ethylcarboxamidocholesterol), I,4-bis(3-N-oleylamino-propyl)piperazine (Gao et al., Biochem Biophys Res Comm. (1991) 179:280; Wolf et al., BioTechniques (1997) 23:139; U.S. Pat. No. 5,744,335), imidazole cholesterol ester (“ICE”; WO2011/068810), sitosterol (22,23-dihydrostigmasterol), β-sitosterol, sitostanol, fucosterol, stigmasterol (stigmasta-5,22-dien-3-ol), ergosterol; desmosterol (3β-hydroxy-5,24-cholestadiene); lanosterol (8,24-lanostadien-3b-ol); 7-dehydrocholesterol (Δ5,7-cholesterol); dihydrolanosterol (24,25-dihydrolanosterol); zymosterol (5α-cholesta-8,24-dien-3β-ol); lathosterol (5α-cholest-7-en-3β-ol); diosgenin ((3β,25R)-spirost-5-en-3-ol); campesterol (campest-5-en-3β-ol); campestanol (5α-campestan-3b-ol); 24-methylene cholesterol (5,24(28)-cholestadien-24-methylen-3β-ol); cholesteryl margarate (cholest-5-en-3β-yl heptadecanoate); cholesteryl oleate; cholesteryl stearate and other modified forms of cholesterol. In some embodiments, the cholesterol-based lipid used in the LNPs is cholesterol.

### D. Helper Lipid

A helper lipid can enhance the structural stability of the LNP and help the LNP in endosome escape. A helper lipid can improve uptake and release of the mRNA drug payload. In some embodiments, the helper lipid is a zwitterionic lipid, which has fusogenic properties for enhancing uptake and release of the drug payload. Examples of helper lipids include, but are not limited to, 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), DMPC, 1,2-dilauroyl-sn-glycero-3-phosphocholine (DLPC), 1,2-Distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), phosphatidylserine, sphingolipids, sphingomyelins, ceramides, cerebrosides, gangliosides, 16-O-monomethyl PE, 16-O-dimethyl PE, 18-1-trans PE, I-stearoyl-2-oleoyl-phosphatidyethanolamine (SOPE), or a combination thereof. In certain embodiments, the helper lipid is DOPE. In certain embodiments, the helper lipid is DSPC.

In various embodiments, the present LNPs comprise (i) a cationic lipid selected from OF-02, cKK-E10, GL-HEPES-E3-E10-DS-3-E18-1, GL-HEPES-E3-E12-DS-4-E10, GL-HEPES-E3-E12-DS-3-E14, or IM-002; (ii) DMG-PEG2000; (iii) cholesterol; and (iv) DOPE.

### E. Molar Ratios of the Lipid Components

The molar ratios of the above components can play a role in the LNPs' effectiveness in delivering mRNA. The molar ratio of the cationic lipid, the PEGylated lipid, the cholesterol-based lipid, and the helper lipid is A: B: C: D, where A+B+C+D=100%. In some embodiments, the molar ratio of the cationic lipid in the LNPs relative to the total lipids (i.e., A) is 35-55%, such as 35-50% (e.g., 38-42% such as 40%, or 45-50%). In some embodiments, the molar ratio of the PEGylated lipid component relative to the total lipids (i.e., B) is 0.25-2.75% (e.g., 1-2% such as 1.5%). In some embodiments, the molar ratio of the cholesterol-based lipid relative to the total lipids (i.e., C) is 20-50% (e.g., 27-30% such as 28.5%, or 38-43%). In some embodiments, the molar ratio of the helper lipid relative to the total lipids (i.e., D) is 5-35% (e.g., 28-32% such as 30%, or 8-12% such as 10%). In some embodiments, the PEGylated lipid+cholesterol components have the same molar amount as the helper lipid. In some embodiments, the LNPs contain a molar ratio of the cationic lipid to the helper lipid that is more than 1.

In certain embodiments, the LNP of the disclosure comprises:
a cationic lipid at a molar ratio of 35% to 55% or 40% to 50% (e.g., a cationic lipid at a molar ratio of 35%, 36%, 37%, 38%, 39%, 40%, 41% 42%, 43%, 44%, 45%, 46%, 47%, 48%, 49%, 50%, 51%, 52%, 53%, 54%, or 55%);
a polyethylene glycol (PEG) conjugated (PEGylated) lipid at a molar ratio of 0.25% to 2.75% or 1.00% to 2.00% (e.g., a PEGylated lipid at a molar ratio of 0.25%, 0.50%, 0.75%, 1.00%, 1.25%, 1.50%, 1.75%, 2.00%, 2.25%, 2.50%, or 2.75%);
a cholesterol-based lipid at a molar ratio of 20% to 50%, 25% to 45%, or 28.5% to 43% (e.g., a cholesterol-based lipid at a molar ratio of 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%); and
a helper lipid at a molar ratio of 5% to 35%, 8% to 30%, or 10% to 30% (e.g., a helper lipid at a molar ratio of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%),
wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%.

In certain embodiments, the PEGylated lipid is dimyristoyl-PEG2000 (DMG-PEG2000).

In various embodiments, the cholesterol-based lipid is cholesterol.

In some embodiments, the helper lipid is 1,2-dioleoyl-SN-glycero-3-phosphoethanolamine (DOPE).

In certain embodiments, the LNP comprises: OF-02 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: cKK-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E10-DS-3-E18-1 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-4-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-3-E14 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: SM-102 at a molar ratio of 35% to 55%; DMG-PEG2000 at a molar ratio of 0.25% to 2.75%; cholesterol at a molar ratio of 20% to 50%; and DSPC at a molar ratio of 5% to 35%.

In certain embodiments, the LNP comprises: ALC-0315 at a molar ratio of 35% to 55%; ALC-0159 at a molar ratio of 0.25% to 2.75%; cholesterol at a molar ratio of 20% to 50%; and DSPC at a molar ratio of 5% to 35%.

In certain embodiments, the LNP comprises: OF-02 at a molar ratio of 40%; DMG-PEG2000 at a molar ratio of 1.5%; cholesterol at a molar ratio of 28.5%; and DOPE at a molar ratio of 30%. This LNP formulation is designated “Lipid A” herein.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%. This LNP formulation is designated “Lipid B” herein.

In certain embodiments, the LNP comprises: GL-HEPES-E3-E10-DS-3-E18-1 at a molar ratio of 40%; DMG-PEG2000 at a molar ratio of 1.5%; cholesterol at a molar ratio of 28.5%; and DOPE at a molar ratio of 30%. This LNP formulation is designated “Lipid C” herein.

In certain embodiments, the LNP comprises: GL-HEPES-E3-E12-DS-4-E10 (at a molar ratio of 40%; DMG-PEG2000 at a molar ratio of 1.5%; cholesterol at a molar ratio of 28.5%; and DOPE at a molar ratio of 30%. This LNP formulation is designated “Lipid D” herein.

In certain embodiments, the LNP comprises: GL-HEPES-E3-E12-DS-3-E14 at a molar ratio of 40%; DMG-PEG2000 at a molar ratio of 1.5%; cholesterol at a molar ratio of 28.5%; and DOPE at a molar ratio of 30%. This LNP formulation is designated “Lipid E” herein.

In certain embodiments, the LNP comprises: 9-heptadecanyl 8-{(2-hydroxyethyl)[6-oxo-6-(undecyloxy)hexyl]amino}octanoate (SM-102) at a molar ratio of 50%; 1,2-distearoyl-sn-glycero-3-phosphocholine (DSPC) at a molar ratio of 10%; cholesterol at a molar ratio of 38.5%; and 1,2-dimyristoyl-rac-glycero-3-methoxypolyethylene glycol-2000 (DMG-PEG2000) at a molar ratio of 1.5%.

In certain embodiments, the LNP comprises: (4-hydroxybutyl)azanediyl]di(hexane-6,1-diyl) bis(2-hexyldecanoate) (ALC-0315) at a molar ratio of 46.3%; 1,2-distearoyl-sn-glycero-3-phosphocholine (DSPC) at a molar ratio of 9.4%; cholesterol at a molar ratio of 42.7%; and 2-[(polyethylene glycol)-2000]-N,N-ditetradecylacetamide (ALC-0159) at a molar ratio of 1.6%.

In certain embodiments, the LNP comprises: (4-hydroxybutyl)azanediyl]di(hexane-6,1-diyl) bis(2-hexyldecanoate) (ALC-0315) at a molar ratio of 47.4%; 1,2-distearoyl-sn-glycero-3-phosphocholine (DSPC) at a molar ratio of 10%; cholesterol at a molar ratio of 40.9%; and 2-[(polyethylene glycol)-2000]-N,N-ditetradecylacetamide (ALC-0159) at a molar ratio of 1.7%.

In certain embodiments, the LNP comprises: IM-001 at a molar ratio of 35% to 55%; a polyethylene glycol (PEG) conjugated (PEGylated) lipid at a molar ratio of 0.25% to 2.75%; a cholesterol-based lipid at a molar ratio of 20% to 45%; and a helper lipid at a molar ratio of 5% to 35%, wherein all of the molar ratios are relative to the total lipid content of the LNP.

In certain embodiments, the LNP comprises: IM-001 at a molar ratio of 40%; a PEGylated lipid at a molar ratio of 1.5%; a cholesterol-based lipid at a molar ratio of 28.5%; and a helper lipid at a molar ratio of 30%, wherein all of the molar ratios are relative to the total lipid content of the LNP.

In certain embodiments, the LNP comprises: IM-001 at a molar ratio of 40%; a DMG-PEG2000 at a molar ratio of 1.5%; a cholesterol-based lipid at a molar ratio of 28.5%; and DOPE at a molar ratio of 30%, wherein all of the molar ratios are relative to the total lipid content of the LNP.

To calculate the actual amount of each lipid to be put into an LNP formulation, the molar amount of the cationic lipid can first be determined based on a desired N/P ratio, where N is the number of nitrogen atoms in the cationic lipid and P is the number of phosphate groups in the mRNA to be transported by the LNP. Next, the molar amount of each of the other lipids can be calculated based on the molar amount of the cationic lipid and the molar ratio selected. These molar amounts can then be converted to weights using the molecular weight of each lipid.

### F. Buffer and Other Components

To stabilize the nucleic acid and/or LNPs (e.g., to prolong the shelf-life of the vaccine product), to facilitate administration of the LNP pharmaceutical composition, and/or to enhance in vivo expression of the nucleic acid, the nucleic acid and/or LNP can be formulated in combination with one or more carriers, targeting ligands, stabilizing reagents (e.g., preservatives and antioxidants), and/or other pharmaceutically acceptable excipients. Examples of such excipients include, but are not limited to, parabens, thimerosal, thiomersal, chlorobutanol, benzalkonium chloride, and chelators (e.g., EDTA).

The LNP compositions of the present disclosure can be provided as a frozen liquid form or a lyophilized form. A variety of cryoprotectants may be used, including, without limitation, sucrose, trehalose, glucose, mannitol, mannose, dextrose, and the like. The cryoprotectant may constitute 5-30% (w/v) of the LNP composition. In some embodiments, the LNP compositions comprise trehalose, e.g., at 5-30% (e.g., 10%) (w/v). Once formulated with the cryoprotectant, the LNP compositions may be frozen (or lyophilized and cryopreserved) at −20° C. to −80° C.

The LNP compositions may be provided to a patient in an aqueous buffered solution—thawed if previously frozen, or if previously lyophilized, reconstituted in an aqueous buffered solution at bedside. The buffered solution can be isotonic and suitable, e.g., for intramuscular or intradermal injection. In some embodiments, the buffered solution is a phosphate-buffered saline (PBS).

### VIII. Vectors

In one aspect, provided herein are vectors comprising the mRNA compositions disclosed herein. The RNA sequences encoding a protein of interest (e.g., mRNA encoding an RSV F protein) can be cloned into a number of types of vectors. For example, the nucleic acids can be cloned into a vector including, but not limited to, a plasmid, a phagemid, a phage derivative, an animal virus, and a cosmid. Suitable vectors can include expression vectors, replication vectors, probe generation vectors, sequencing vectors, and vectors optimized for in vitro transcription.

In certain embodiments, the vector can be used to express mRNA in a host cell. In various embodiments, the vector can be used as a template for IVT. The construction of optimally translated IVT mRNA suitable for therapeutic use is disclosed in detail in Sahin, et al. (2014). Nat. Rev. Drug Discov. 13, 759-780; Weissman (2015). Expert Rev. Vaccines 14, 265-281.

In some embodiments, the vectors disclosed herein can comprise at least the following, from 5′ to 3′: an RNA polymerase promoter; a polynucleotide sequence encoding a 5′ UTR; a polynucleotide sequence encoding an ORF; a polynucleotide sequence encoding a 3′ UTR; and a polynucleotide sequence encoding at least one RNA aptamer. In some embodiments, the vectors disclosed herein may comprise a polynucleotide sequence encoding a poly(A) sequence and/or a polyadenylation signal.

A variety of RNA polymerase promoters are known. In some embodiments, the promoter can be a T7 RNA polymerase promoter. Other useful promoters can include, but are not limited to, T3 and SP6 RNA polymerase promoters. Consensus nucleotide sequences for T7, T3, and SP6 promoters are known.

Also disclosed herein are host cells (e.g., mammalian cells, e.g., human cells) comprising the vectors or RNA compositions disclosed herein.

Polynucleotides can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, MA) or the Gene Pulser II (BioRad, Denver, CO), Multiporator (Eppendorf, Hamburg, Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12(8):861-70, or the TransIT-RNA transfection Kit (Mirus, Madison, WI)).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present disclosure, in order to confirm the presence of the mRNA sequence in the host cell a variety of assays may be performed.

### IX. Self-Replicating RNA and Trans-Replicating RNA

### Self-Replicating RNA:

In one aspect, disclosed herein are self-replicating RNAs encoding an RSV F protein.

Self-replicating RNA can be produced by using replication elements derived from, e.g., alphaviruses, and substituting the structural viral proteins with a nucleotide sequence encoding a protein of interest (e.g., RSV F protein). A self-replicating RNA is typically a positive-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen (i.e., an RSV F protein antigen), or may be transcribed to provide further transcripts with the same sense as the delivered RNA which are translated to provide in situ expression of the antigen. The overall result of this sequence of transcriptions is a large amplification in the number of the introduced replicon RNAs and so the encoded antigen becomes a major polypeptide product of the cells.

One suitable system for achieving self-replication in this manner is to use an alphavirus-based replicon. These replicons are positive stranded (positive sense-stranded) RNAs which lead to translation of a replicase (or replicase-transcriptase) after delivery to a cell. The replicase is translated as a polyprotein which auto-cleaves to provide a replication complex which creates genomic-strand copies of the positive-strand delivered RNA. These negative (−)-stranded transcripts can themselves be transcribed to give further copies of the positive-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used, e.g., the attenuated TC83 mutant of VEEV has been used in replicons, see the following reference: WO2005/113782, incorporated herein by reference.

In one embodiment, each self-replicating RNA described herein encodes (i) an RNA-dependent RNA polymerase which can transcribe RNA from the self-replicating RNA molecule and (ii) an RSV F protein antigen. The polymerase can be an alphavirus replicase, e.g., comprising one or more of alphavirus proteins nsP1, nsP2, nsP3, and nsP4. Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins. Self-replicating RNA are described in further detail in WO2011005799, incorporated herein by reference.

### Trans-Replicating RNA:

In one aspect, disclosed herein are trans-replicating RNAs encoding an RSV F protein.

Trans-replicating RNA possess similar elements as the self-replicating RNA described above. However, with trans-replicating RNA, two separate RNA molecules are used. A first RNA molecule encodes for the RNA replicase described above (e.g., the alphavirus replicase) and a second RNA molecule encodes for the protein of interest (e.g., an RSV F protein antigen). The RNA replicase may replicate one or both of the first and second RNA molecule, thereby greatly increasing the copy number of RNA molecules encoding the protein of interest. Trans replicating RNA are described in further detail in WO2017162265, incorporated herein by reference.

### X. Processes for Making LNP Vaccines

The present LNPs can be prepared by various techniques. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion that results in the formation of MLVs. Unilamellar vesicles (ULV) can then be formed by homogenization, sonication, or extrusion of the multilamellar vesicles. In addition, unilamellar vesicles can be formed by detergent removal techniques.

Various methods are described in US 2011/0244026, US 2016/0038432, US 2018/0153822, US 2018/0125989, and US 2021/0046192 and can be used for making LNP vaccines. One exemplary process entails encapsulating mRNA by mixing it with a mixture of lipids, without first pre-forming the lipids into lipid nanoparticles, as described in US 2016/0038432. Another exemplary process entails encapsulating mRNA by mixing pre-formed LNPs with mRNA, as described in US 2018/0153822.

In some embodiments, the process of preparing mRNA-loaded LNPs includes a step of heating one or more of the solutions to a temperature greater than ambient temperature, the one or more solutions being the solution comprising the pre-formed lipid nanoparticles, the solution comprising the mRNA, and the mixed solution comprising the LNP-encapsulated mRNA. In some embodiments, the process includes the step of heating one or both of the mRNA solution and the pre-formed LNP solution prior to the mixing step. In some embodiments, the process includes heating one or more of the solutions comprising the pre-formed LNPs, the solution comprising the mRNA, and the solution comprising the LNP-encapsulated mRNA during the mixing step. In some embodiments, the process includes the step of heating the LNP-encapsulated mRNA after the mixing step. In some embodiments, the temperature to which one or more of the solutions is heated is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature is about 65° C.

Various methods may be used to prepare an mRNA solution suitable for the present disclosure. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water or a buffer at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include, but are not limited to, gear pumps, peristaltic pumps, and centrifugal pumps. Typically, the buffer solution is mixed at a rate greater than that of the mRNA stock solution. For example, the buffer solution may be mixed at a rate at least 1x, 2x, 3x, 4x, 5x, 6x, 7x, 8x, 9x, 10x, 15x, or 20× greater than the rate of the mRNA stock solution. In some embodiments, a buffer solution is mixed at a flow rate ranging from about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of, or greater than, about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging from about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of, or greater than, about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

The process of incorporation of a desired mRNA into a lipid nanoparticle is referred to as “loading.” Exemplary methods are described in Lasic et al., FEBS Lett. (1992) 312:255-8. The LNP-incorporated nucleic acids may be completely or partially located in the interior space of the lipid nanoparticle, within the bilayer membrane of the lipid nanoparticle, or associated with the exterior surface of the lipid nanoparticle membrane. The incorporation of an mRNA into lipid nanoparticles is also referred to herein as “encapsulation” wherein the nucleic acid is entirely or substantially contained within the interior space of the lipid nanoparticle.

Suitable LNPs may be made in various sizes. In some embodiments, decreased size of lipid nanoparticles is associated with more efficient delivery of an mRNA. Selection of an appropriate LNP size may take into consideration the site of the target cell or tissue and to some extent the application for which the lipid nanoparticle is being made.

A variety of methods are available for sizing of a population of lipid nanoparticles. In various embodiments, methods herein utilize Zetasizer Nano ZS (Malvern Panalytical) to measure LNP particle size. In one protocol, 10 μl of an LNP sample are mixed with 990 μl of 10% trehalose. This solution is loaded into a cuvette and then put into the Zetasizer machine. The z-average diameter (nm), or cumulants mean, is regarded as the average size for the LNPs in the sample. The Zetasizer machine can also be used to measure the polydispersity index (PDI) by using dynamic light scattering (DLS) and cumulant analysis of the autocorrelation function. Average LNP diameter may be reduced by sonication of formed LNP. Intermittent sonication cycles may be alternated with quasi-elastic light scattering (QELS) assessment to guide efficient lipid nanoparticle synthesis.

In some embodiments, the majority of purified LNPs, i.e., greater than about 50%, 55%, 60%, 65%, 70%, 75%, 80%, 85%, 90%, 95%, 96%, 97%, 98%, or 99% of the LNPs, have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all (e.g., greater than 80% or 90%) of the purified lipid nanoparticles have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In certain embodiments, the LNP has an average diameter of 30-200 nm.

In various embodiments, the LNP has an average diameter of 80-150 nm.

In some embodiments, the LNPs in the present composition have an average size of less than 150 nm, less than 120 nm, less than 100 nm, less than 90 nm, less than 80 nm, less than 70 nm, less than 60 nm, less than 50 nm, less than 30 nm, or less than 20 nm.

In some embodiments, greater than about 70%, 75%, 80%, 85%, 90%, 95%, 96%, 97%, 98%, or 99% of the LNPs in the present composition have a size ranging from about 40-90 nm (e.g., about 45-85 nm, about 50-80 nm, about 55-75 nm, or about 60-70 nm) or about 50-70 nm (e.g., about 55-65 nm) are suitable for pulmonary delivery via nebulization.

In some embodiments, the dispersity, or measure of heterogeneity in size of molecules (PDI), of LNPs in a pharmaceutical composition provided by the present disclosure is less than about 0.5. In some embodiments, an LNP has a PDI of less than about 0.5, less than about 0.4, less than about 0.3, less than about 0.28, less than about 0.25, less than about 0.23, less than about 0.20, less than about 0.18, less than about 0.16, less than about 0.14, less than about 0.12, less than about 0.10, or less than about 0.08. The PDI may be measured by a Zetasizer machine as described above.

In some embodiments, greater than about 75%, 80%, 85%, 90%, 95%, 96%, 97%, 98%, or 99% of the purified LNPs in a pharmaceutical composition provided herein encapsulate an mRNA within each individual particle. In some embodiments, substantially all (e.g., greater than 80% or 90%) of the purified lipid nanoparticles in a pharmaceutical composition encapsulate an mRNA within each individual particle. In some embodiments, a lipid nanoparticle has an encapsulation efficiency of 50% to 99%; or greater than about 60%, 65%, 70%, 75%, 80%, 85%, 90%, 92%, 95%, 98%, or 99%. Typically, lipid nanoparticles for use herein have an encapsulation efficiency of at least 90% (e.g., at least 91%, 92%, 93%, 94%, or 95%).

In some embodiments, an LNP has a N/P ratio of 1 to 10. In some embodiments, a lipid nanoparticle has a N/P ratio above 1, about 1, about 2, about 3, about 4, about 5, about 6, about 7, or about 8. In certain embodiments, a typical LNP herein has an N/P ratio of 4.

In some embodiments, a pharmaceutical composition according to the present disclosure contains at least about 0.5 μg, 1 μg, 5 μg, 10 μg, 100 μg, 500 μg, or 1000 μg of encapsulated mRNA. In some embodiments, a pharmaceutical composition contains about 0.1 μg to 1000 μg, at least about 0.5 μg, at least about 0.8 μg, at least about 1 μg, at least about 5 μg, at least about 8 μg, at least about 10 μg, at least about 50 μg, at least about 100 μg, at least about 500 μg, or at least about 1000 μg of encapsulated mRNA.

In some embodiments, mRNA can be made by chemical synthesis or by in vitro transcription (IVT) of a DNA template. In this process, an IVT process, a cDNA template is used to produce an mRNA transcript and the DNA template is degraded by a DNase. The transcript is purified by depth filtration and tangential flow filtration (TFF). The purified transcript is further modified by adding a cap and a tail, and the modified RNA is purified again by depth filtration and TFF.

The mRNA is then prepared in an aqueous buffer and mixed with an amphiphilic solution containing the lipid components of the LNPs. An amphiphilic solution for dissolving the four lipid components of the LNPs may be an alcohol solution. In some embodiments, the alcohol is ethanol. The aqueous buffer may be, for example, a citrate, phosphate, acetate, or succinate buffer and may have a pH of about 3.0-7.0, e.g., about 3.5, about 4.0, about 4.5, about 5.0, about 5.5, about 6.0, or about 6.5. The buffer may contain other components such as a salt (e.g., sodium, potassium, and/or calcium salts). In certain embodiments, the aqueous buffer has 1 mM citrate and 150 mM NaCl at pH 4.5.

An exemplary, nonlimiting process for making an mRNA-LNP composition involves mixing a buffered mRNA solution with a solution of lipids in ethanol in a controlled homogeneous manner, where the ratio of lipids:mRNA is maintained throughout the mixing process. In this illustrative example, the mRNA is presented in an aqueous buffer containing citric acid monohydrate, tri-sodium citrate dihydrate, and sodium chloride. The mRNA solution is added to the solution (1 mM citrate buffer, 150 mM NaCl, pH 4.5). The lipid mixture of four lipids (e.g., a cationic lipid, a PEGylated lipid, a cholesterol-based lipid, and a helper lipid) is dissolved in ethanol. The aqueous mRNA solution and the ethanol lipid solution are mixed at a volume ratio of 4:1 in a “T” mixer with a near “pulseless” pump system. The resultant mixture is then subjected for downstream purification and buffer exchange. The buffer exchange may be achieved using dialysis cassettes or a TFF system. TFF may be used to concentrate and buffer-exchange the resulting nascent LNP immediately after formation via the T-mix process. The diafiltration process is a continuous operation, keeping the volume constant by adding appropriate buffer at the same rate as the permeate flow.

### XI. Packaging and Use of the mRNA-LNP RSV Vaccine

The mRNA-LNP vaccines can be formulated or packaged for parenteral (e.g., intramuscular, intradermal, or subcutaneous) administration or nasopharyngeal (e.g., intranasal) administration. In various embodiments, the mRNA-LNP vaccines may be formulated or packaged for pulmonary administration. In various embodiments, the mRNA-LNP vaccines may be formulated or packaged for intravenous administration. The vaccine compositions may be in the form of an extemporaneous formulation, where the LNP composition is lyophilized and reconstituted with a physiological buffer (e.g., PBS) just before use. The vaccine compositions also may be shipped and provided in the form of an aqueous solution or a frozen aqueous solution and can be directly administered to subjects without reconstitution (after thawing, if previously frozen).

Accordingly, the present disclosure provides an article of manufacture, such as a kit, that provides the mRNA-LNP vaccine in a single container or provides the mRNA-LNP vaccine in one container (e.g., a first container) and a physiological buffer for reconstitution in another container (e.g., a second container). The container(s) may contain a single-use dosage or multi-use dosage. The container(s) may be pre-treated glass vials or ampules. The article of manufacture may include instructions for use as well.

In certain embodiments, the mRNA-LNP vaccine is provided for use in intramuscular (IM) injection. The vaccine can be injected into a subject at, e.g., at his/her deltoid muscle in the upper arm. In some embodiments, the vaccine is provided in a pre-filled syringe or injector (e.g., single-chambered or multi-chambered). In some embodiments, the vaccine is provided for use in inhalation and is provided in a pre-filled pump, aerosolizer, or inhaler.

The mRNA-LNP vaccines can be administered to subjects in need thereof in a prophylactically effective amount, i.e., an amount that provides sufficient immune protection against a target pathogen for a sufficient amount of time (e.g., one year, two years, five years, ten years, or a lifetime). Sufficient immune protection may be, for example, prevention or alleviation of symptoms associated with infections by the pathogen. In some embodiments, multiple doses (e.g., two doses) of the vaccine are administered (e.g., injected) to subjects in need thereof to achieve the desired prophylactic effects. The doses (e.g., prime and booster doses) may be separated by an interval of at least, e.g., 2 weeks, 3 weeks, 4 weeks, one month, two months, three months, four months, five months, six months, one year (i.e., twelve months), two years, five years, or ten years.

In order that this invention may be better understood, the following examples are set forth. These examples are for purposes of illustration only and are not to be construed as limiting the scope of the invention in any manner.

### EXAMPLES

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

### Example 1: A Phase I/II, Randomized, Double-Blind, Placebo-Controlled Multi-Arm Dose-Finding Study to Evaluate the Safety and Immunogenicity of an RSV mRNA Vaccine Candidate with Either LNP cKK-E10 or LNP GL-HEPES-E3-E12-DS-4-E10 in Adult Participants 18 to 50 Years of Age in Study A (Sentinel Cohort) and 60 Years of Age and Older in Study B and Booster Study (Main and Booster Cohorts)

### Introduction

### Background

There are currently no vaccines available for the prevention of RSV in older adults, and there are no effective antiviral treatments. Therefore, there is an unmet medical need to address the prevention of respiratory disease in older adults while improving patient quality of life.

This example outlines the parameters used to evaluate the efficacy, safety, and immunogenicity of RSV mRNA LNP vaccines as described herein in adults (18 to 50 years of age and 60 years of age and older). Such RSV mRNA LNP vaccines may prevent LRTD caused by RSV in older adults.

### Study Rationale

The clinical trials described herein test the safety and immunogenicity of the RSV mRNA LNP vaccine. The RSV mRNA LNP vaccine comprises an mRNA encoding the RSV pre-fusion (pre-F) antigen in one of two encapsulated LNPs formulations (i.e., an LNP containing either cKK-E10 (non-biodegradable) or GL-HEPES-E3-E12-DS-4-E10 (biodegradable)) administered at three different doses (i.e., low dose (10 μg), medium dose (30 μg), or high dose (75 μg)) in healthy adults aged 18 to 50 years (Study A, i.e., Sentinel Cohort) and 60 years and older (Study B and Booster Study (i.e., Main and Booster Cohorts)).

### Study Overview and Study Design

### Number of Participants, Intervention Groups Overview, and Visit Frequency

The total anticipated number of participants across the two studies (Study A and Study B) is approximately 790 randomized participants.

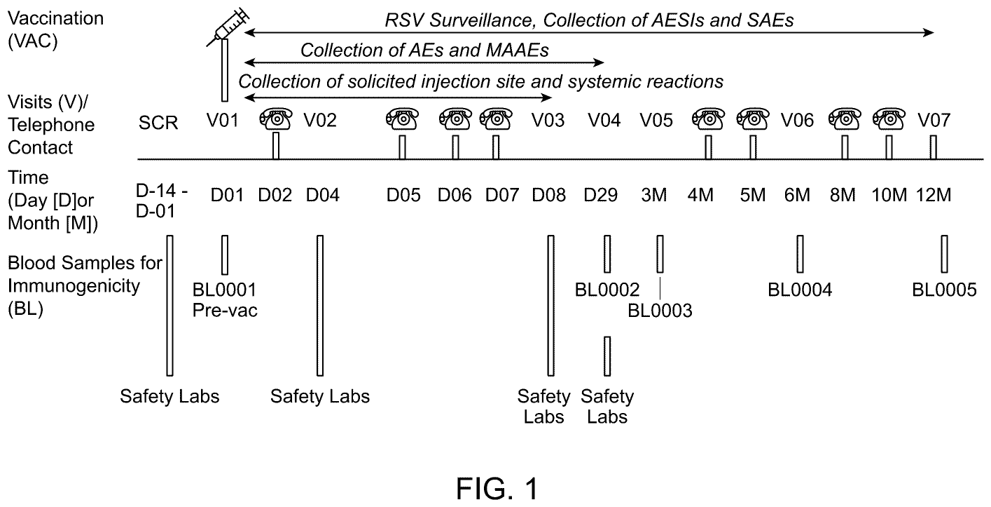

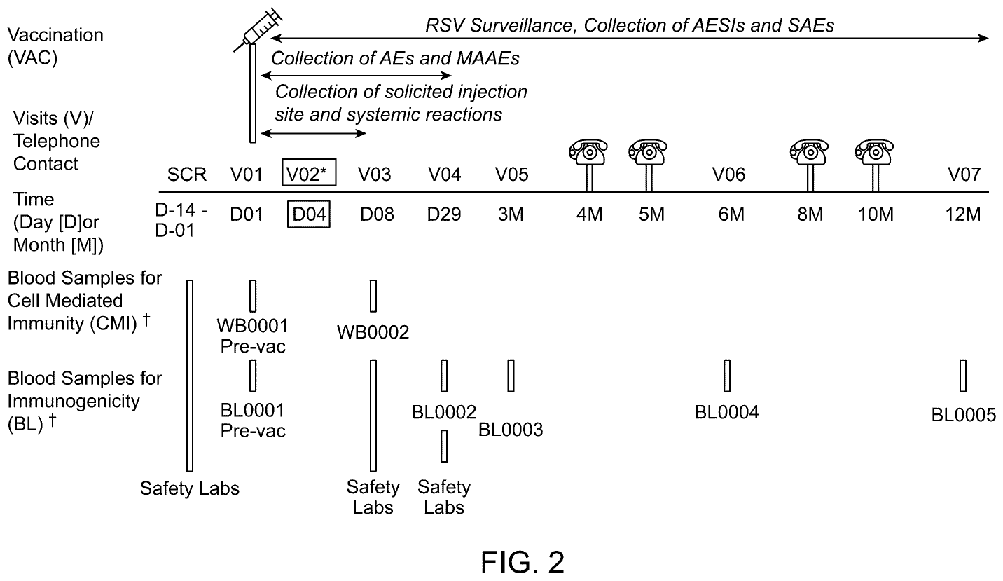

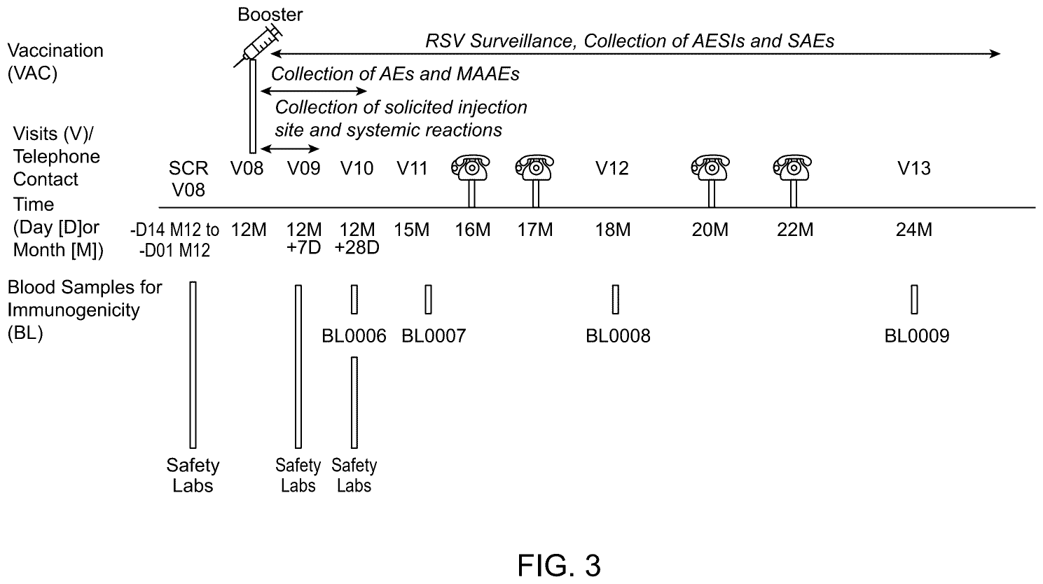

Study A (entitled, “Sentinel Cohort”), the initial clinical trial, is a smaller randomized, double-blind, dose-escalation safety study (i.e., about 90 participants total, see Table 2 below) which will be followed by a larger Study B (entitled, “Main Cohort”; about 700 participants total; see Table 3 below) to evaluate the safety and immunogenicity of an RSV mRNA vaccine encapsulated in an LNP in healthy adult participants (18 to 50 years of age in Study A; 60 years and older in study B). Graphical timelines of Study A and Study B are shown in FIG. 1 and FIG. 2, respectively.

Studies A and B comprise the same six experimental subcohorts as well as a placebo control group. The six experimental subcohorts are administered 3 different doses (i.e., low dose (10 μg), medium dose (30 μg), and high dose (75 μg)) of the RSV messenger mRNA vaccine candidate (set forth as SEQ ID NO: 14) encapsulated in either of two different lipid nanoparticle (LNP)-based formulations (i.e., LNP containing cKK-E10 or LNP GL-HEPES-E3-E12-DS-4-E10). Both Studies A and B will also have a placebo control group which will be administered a 0.9% normal saline solution. The vaccine is administered intramuscularly (deltoid muscle in the upper arm) at a dosage level of 0.5 ml per dose. Vaccines are stored at −80° C.±10° C. and diluted with 2.2×PBS at the study site.

Upon vaccination, participants in Studies A and B are followed for 12-months post-vaccination. Study A has an initial screening participant visit plus 7 planned site visits occurring at Day (D) −14 (−D14), D01, D04, D08, D29, 3 months, 6 months, and 12 months. Study B has an initial screening participant visit plus 6 planned site visits occurring at −D14, D01, D08, D29, 3 months, 6 months, and 12 months.

It is also planned for 140 participants (i.e., 20 participants per arm) enrolled in Study B to be selected for inclusion in the cell-mediated immunity (CMI) subset. Participants in the CMI subset will be enrolled from a limited number of selected sites. It is also planned in Study B to enroll a minimum of 6% participants of Japanese origin (i.e., minimum of approximately 42 participants; 6 per arm).

TABLE 2
Experimental Sample Size for Study A (Sentinel Cohort)

| Sub- | | | | Unit | |
|---|---|---|---|---|---|
| cohort | | | Dose | Dose | |
| Number | Group | Study Intervention | Level | (μg) | N |
| 1 | 1 | RSV mRNA LNP cKK-E10 | Low | 10 | 10 |
| | 2 | RSV mRNA LNP GL-HEPES- | | 10 | 10 |
| | | E3-E12-DS-4-E10 | | | |
| 2 | 3 | RSV mRNA LNP cKK-E10 | Medium | 30 | 10 |
| | 4 | RSV mRNA LNP GL-HEPES- | | 30 | 10 |
| | | E3-E12-DS-4-E10 | | | |
| 3 | 5 | RSV mRNA LNP cKK-E10 | High | 75 | 10 |
| | 6 | RSV mRNA LNP GL-HEPES- | | 75 | 10 |
| | | E3-E12-DS-4-E10 | | | |
| 1, 2, 3 | 7 | Placebo | N/A | N/A | 30 |

LNP: lipid nanoparticle; mRNA: messenger ribonucleic acid; N/A: not applicable; RSV: respiratory syncytial virus; RSV mRNA is set forth as SEQ ID NO: 14.

TABLE 3
Experimental Sample Size for Study B (Main Cohort)

| | | Dose | Unit Dose | |
|---|---|---|---|---|
| Group | Study Intervention | Level | (μg) | N |
| 1 | RSV mRNA LNP cKK-E10 | Low | 10 | 100 |
| 2 | RSV mRNA LNP GL-HEPES- | Low | 10 | 100 |
| | E3-E12-DS-4-E10 | | | |
| 3 | RSV mRNA LNP cKK-E10 | Medium | 30 | 100 |
| 4 | RSV mRNA LNP GL-HEPES- | Medium | 30 | 100 |
| | E3-E12-DS-4-E10 | | | |
| 5 | RSV mRNA LNP cKK-E10 | High | 75 | 100 |
| 6 | RSV mRNA LNP GL-HEPES- | High | 75 | 100 |
| | E3-E12-DS-4-E10 | | | |
| 7 | Placebo | N/A | N/A | 100 |

LNP: lipid nanoparticle; mRNA: messenger ribonucleic acid; N/A: not applicable; RSV: respiratory syncytial virus; RSV mRNA is set forth as SEQ ID NO: 14.

In addition, these clinical trials evaluate the safety and immunogenicity of a booster vaccination administered 12 months after the primary vaccination in a subset of the study population (entitled, “Booster Cohort”; approximately 200 participants from Study B; see Table 4 below). A booster vaccination is administered 12 months after the primary vaccination in a subset of the study population from Study B. For the booster vaccination, a single vaccine formulation is used, based on the evaluation of candidate formulations after the first injection. The duration of each participant's participation is 24 months overall for the subset of participants enrolled in the Booster Cohort. Similar to Study B, participants have a screening visit in addition to 6 planned visits. Participants receive a booster vaccination 12 months post-primary vaccination at the 8^{th} visit, which may take place on the same day as the 12-month Study B follow-up visit (i.e., visit 7). Following the booster vaccination, participants return to the site at D08, D29, 3 months, 6 months, and 12 months.

TABLE 4
Experimental Sample Size for Booster Cohort

| | Study | | Unit Dose | |
|---|---|---|---|---|
| Group | Intervention | Dose Level | (μg) | N |
| 1, 2, 3, 4, | Selected | Low, Medium, | Selected | 100† |
| 5, or 6 | Formulation* | or High | Dose* | |
| 7 | Placebo | N/A | N/A | 100† |

*The dose level and study intervention formulation is determined at the time of interim analysis.
†N is defined here without considering the drop-out rate at the time of booster vaccination.
Participants in the Booster Cohort will be randomized to receive either a booster dose of the selected formulation or placebo.

### End of Study Definition

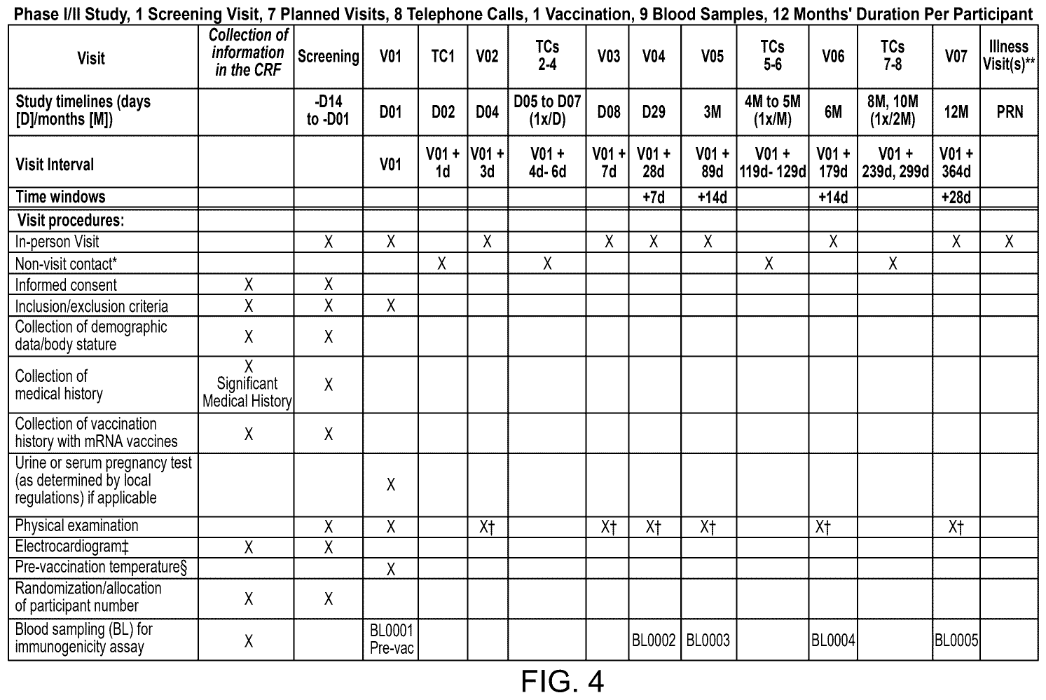

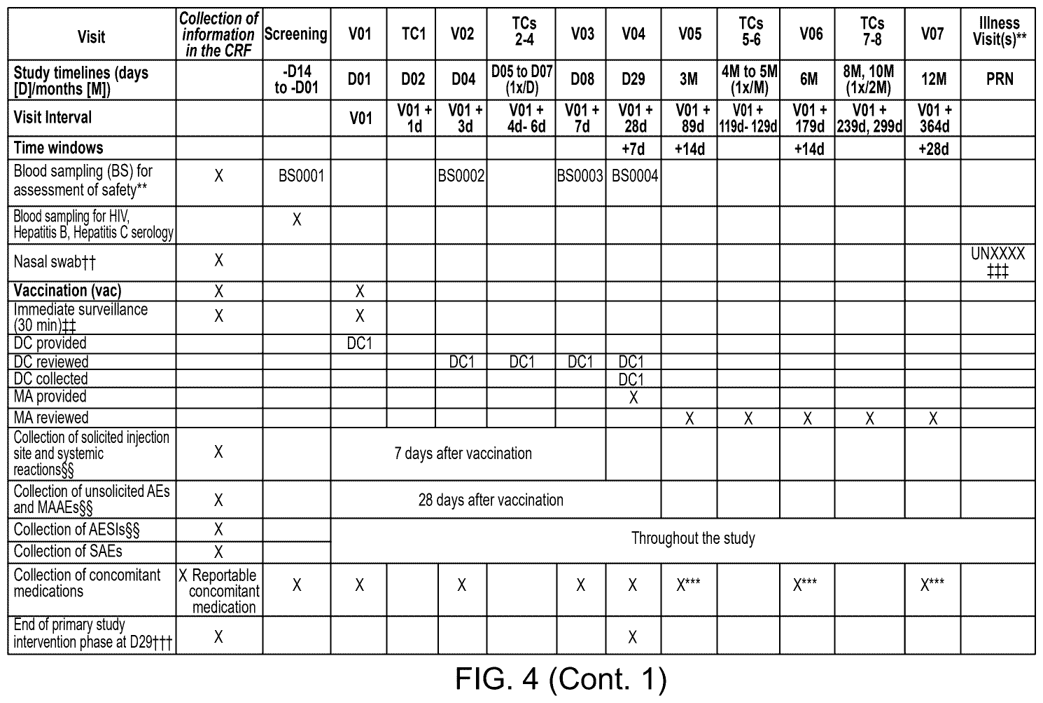

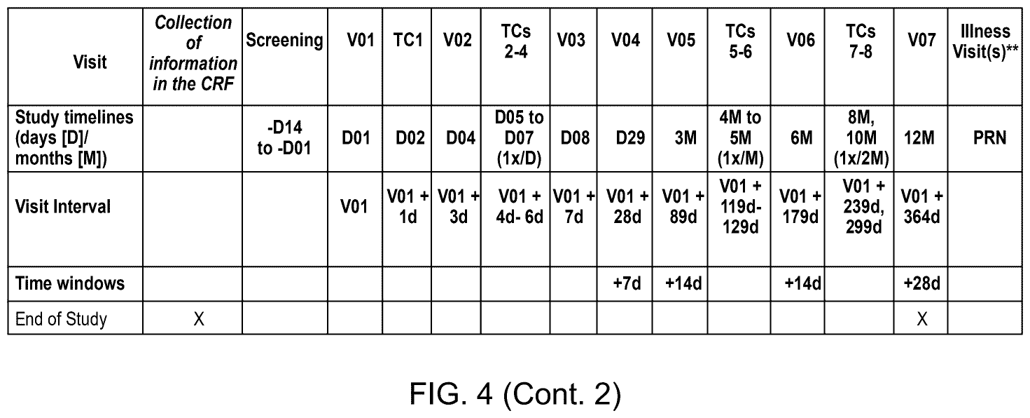

A participant is considered to have completed the study if they have completed the last contact planned in the Scheduled Activities. Scheduled activities for Study A, B, and the Booster Cohort are shown in FIG. 4-FIG. 6. The end of the study is defined as the date of the last contact of the last participant in the study. However, for periodic safety reports, the study is considered completed when the clinical study report is finalized.

### Dosing

The vaccine is provided as a liquid frozen solution in a vial. Each 0.5 mL dose contains: 10 μg, 30 μg, or 75 μg of RSV pre-F mRNA; and LNP containing cKK-E10 or LNP containing GL-HEPES-E3-E12-DS-4-E10. The vaccine is formulated as a single dose of an mRNA-LNP complex that is diluted to the required mRNA dose at the study site using a buffer diluent (diluent=2.2×PBS (2° C. to 8° C.)). The sentinel and main cohorts administer one intramuscular injection. The booster cohort utilizes two intramuscular injections, with the second injection administered 12 months post-primary injection. Each dose (vial) of vaccine is provided in an individual box. Each dose (vial) of vaccine is stored at −80° C.+/−10° C.

### Objectives

Primary Objectives. The primary objectives are to assess the safety and immunogenicity profile of the three different dose-levels (i.e., low dose (10 μg), medium dose (30 μg), and high dose (75 μg)) of the RSV mRNA vaccine described herein encapsulated in either an LNP comprising cKK-E10 or in an LNP comprising GL-HEPES-E3-E12-DS-4-E10.

Secondary Objectives. The secondary objectives are to assess: (1) the safety profile of a booster vaccination given 12 months after the primary vaccination, in a subset of participants; (2) the durability of immune response at 3, 6, and 12 months following primary vaccination on pre-vaccination (D01); and (3) the durability of the immune response following booster vaccination 12 months after the primary vaccination, in a subset of participants.

Table 5 below summarizes the primary objectives and the corresponding endpoints. Table 6 below summarizes the secondary objectives and the corresponding endpoints. Table 7 below summarizes the exploratory objectives and the corresponding endpoints.

TABLE 5
Primary Objectives and Corresponding Endpoints

| | |
|---|---|
| Objectives | Endpoints |

Primary

| Safety Objective | Safety Endpoints |
|---|---|
| To assess the safety profile of 3 different | Presence of unsolicited systemic adverse |
| dose-levels of RSV mRNA vaccine | event (AE) reported in the 30 minutes after |
| candidate with an LNP containing cKK- | vaccination |
| E10 and RSV mRNA vaccine candidate | Presence of solicited injection site and systemic |
| with an LNP containing GL-HEPES-E3- | reactions occurring up to 7 days after |
| E12-DS-4-E10 | vaccination |
| | Presence of unsolicited adverse event or |
| | medically attended adverse event (MAAE) |
| | reported up to 28 days after vaccination |
| | Presence of serious adverse events, and AEs of |
| | special interest (AESIs) throughout the study |
| | Presence of out-of-range biological test results up |
| | to 7 days after vaccination |
| Immunogenicity Objective | Immunogenicity Endpoint |
| To assess the immunogenicity of 3 | RSV-A serum neutralizing antibody (nAb) titers at |
| different dose-levels of RSV mRNA | pre-vaccination (D 01) and 28 days post-primary |
| vaccine candidate encapsulated in an LNP | vaccination (D 29) |
| containing cKK-E10 and RSV mRNA | |
| vaccine candidate encapsulated in an LNP | |
| containing GL-HEPES-E3-E12-DS-4-E10 | |

TABLE 6
Secondary Objectives and Corresponding Endpoints

| | |
|---|---|
| Objectives | Endpoints |

Secondary

| Safety Objective | | Safety Endpoints |
|---|---|---|
| To assess safety profile of a | | Presence of immediate unsolicited systemic AEs |
| booster vaccination given 12 | | reported in the 30 minutes after booster |
| months after the primary | | vaccination |
| vaccination, in a subset of | | Presence of solicited injection site and |
| participants | | systemic reactions occurring up to 7 days |
| | | after booster vaccination |
| | | Presence of unsolicited AEs and MAAEs reported |
| | | up to 28 days after booster vaccination |
| | | Presence of SAEs and AESIs throughout the study |
| | | Presence of out-of-range biological test results up |
| | | to 7 days after booster vaccination |
| Immunogenicity Objectives | | Immunogenicity Endpoints |
| 1) | To assess the durability of immune | Endpoints for objective #1: |
| | response at 3, 6, and 12 months | RSV-A serum nAb titers at pre-vaccination |
| | following primary vaccination on D 01 | (D 01), 28 days (D 29), and 3-, 6-, and 12- |
| 2) | To assess the durability of the immune | months post-primary vaccination |
| | response following booster vaccination | serum anti-F immunoglobulin G (IgG) Ab titers at |
| | 12 months after the primary vaccination, | pre-vaccination (D 01), 28 days (D 29), and 3-, 6-, |
| | in a subset of participants | and 12-months post-primary vaccination |
| | | Endpoints for objective #2: |
| | | RSV-A serum nAb titers at pre-booster |
| | | vaccination, 28 days, and 3-, 6-, and 12-months |
| | | post-booster vaccination |
| | | Serum anti-F IgG Ab titers at pre-booster |
| | | vaccination, 28 days, and 3-, 6-, and 12- |
| | | months post-booster vaccination |

TABLE 7
Exploratory Objectives and Corresponding Endpoints

| Exploratory Objective | Exploratory Endpoint |
|---|---|
| To assess the immunogenicity of an RSV | RSV-B serum nAb titers at pre-vaccination |
| mRNA vaccine candidate at low, medium, | (D 01) and 28 days (D 29) post-primary |
| and high dose in an LNP containing cKK- | vaccination |
| E10 or LNP GL-HEPES-E3-E12-DS-4- | |
| E10 | |
| To assess the durability of the immune | RSV-B serum nAb titers at pre-vaccination |
| response following primary vaccination on | (D 01), 28 days post-primary vaccination |
| D 01 | (D 29), and 3, 6, and 12 months post-primary |
| | vaccination |
| To assess the durability of the immune | RSV-B serum nAb titers at pre-booster |
| response following booster vaccination 12 | vaccination, 28 days, and 3-, 6-, and 12- |
| months after the primary vaccination, in a | months post-booster vaccination |
| subset of participants | |
| To describe the frequency of RSV-associated | Incidence of laboratory confirmed LRTD for |
| LRTD | any RSV strain (RSV-A and RSV-B) |
| To describe the frequency of RSV-associated | Incidence of laboratory confirmed ARD for |
| acute respiratory disease (ARD) | any RSV strain (RSV-A and RSV- B) |
| Exploration of site-specific immune response | Competitive Ab titers against site Ø, I, and II |
| to RSV | |
| Exploration of T-cell response to the 3- | Exploratory CMI assays at D 01 and D 08 for a |
| vaccine dose-levels in a subset of 140 | subset of the population |
| participants | |
| To describe the frequency of other viral | Incidence of laboratory confirmed LRTD for |
| etiologies associated with LRTD | any other viral etiology detected by |
| | GenMarkDx test |
| To describe the frequency of other viral | Incidence of laboratory confirmed ARD for |
| etiologies associated with ARD | any other viral etiology detected by |
| | GenMarkDx test |
| To characterize the RSV A serum | RSV A serum neutralizing antibody titers by |
| neutralizing antibody responses to the study | D 29 converted into international units (IU/mL) |
| product converted to IU/mL values in each | |
| vaccine group after primary vaccination (D 29) | |
| To characterize the RSV B serum | RSV B serum neutralizing antibody titers by |
| neutralizing antibody responses to the study | D 29 converted into international units (IU/mL) |
| product converted to IU/mL values in each | |
| vaccine group after primary vaccination (D 29) | |

### Study Population

### Inclusion and Exclusion Criteria

Inclusion Criteria. For all studies (Study A, B, and Booster Cohort) participants are eligible to be included in the study only if all of the following criteria apply at time of screening and at the first visit (Day 01; Visit 01): (11) a participant must be (1) aged 18 to 50 years on the day of inclusion (i.e., “18 years of age” means from the day of the 18^{th} birthday) for Study A or (2) 60 years of age or older on the day of inclusion (i.e., “60 years of age or older” means from the day of the 60^{th} birthday) for Study B and Booster Cohort; (12) a female participant is eligible to participate if she is not pregnant or breastfeeding and is of non-childbearing potential (to be considered of non-childbearing potential, a female must be postmenopausal for at least 1 year or surgically sterile). For the Sentinel Cohort, urine or serum pregnancy testing will be performed in women of childbearing potential before vaccination; and (13) a participant must be able to attend all scheduled visits and to comply with all study procedures. Also, a fourth additional criteria at the time of screening is that the participant sign and date the informed consent form.

Exclusion Criteria. For all studies (Study A, B, and Booster Cohort) participants are not eligible if any of the following criteria are met: (E1) known or suspected congenital or acquired immunodeficiency; or receipt of immunosuppressive therapy, such as anti-cancer chemotherapy or radiation therapy, within the preceding 6 months; or long-term systemic corticosteroid therapy (prednisone or equivalent for more than 2 consecutive weeks within the past 3 months); (E2) known systemic hypersensitivity to any of the study intervention components (e.g., polyethylene glycol, polysorbate); history of a life-threatening reaction to the study interventions used in the study or to a product containing any of the same substances, any allergic reaction (e.g., anaphylaxis) after administration of mRNA COVID-19 vaccine; (E3) history of RSV-associated illness, diagnosed clinically, serologically, or microbiologically in the last 12 months; (E4) previous history of myocarditis, pericarditis, and/or myopericarditis; (E5) thrombocytopenia or bleeding disorder, contraindicating IM injection based on Investigator's judgment; (E6) bleeding disorder, or receipt of anticoagulants in the 3 weeks preceding inclusion, contraindicating intramuscular injection; (E7) chronic illness that, in the opinion of the investigator, is at a stage where it might interfere with study conduct or completion (e.g., cardiac disorders, renal disorders, auto-immune disorders, diabetes, psychiatric disorders, or chronic infection); (E8) alcohol, prescription drug, or substance abuse that, in the opinion of the Investigator, might interfere with the study conduct or completion; (E9) receipt of any vaccine in the 4 weeks preceding any study intervention administration or planned receipt of any vaccine in the 4 weeks following any study intervention administration (if the participant is enrolled and seeks vaccination of an authorized influenza or non-mRNA COVID-19 vaccine outside of the study, he/she will be encouraged to discuss this intention proactively with the study Investigator and will be permitted to receive the authorized vaccine at the earliest 28 days after study vaccination and at any time thereafter); (E10) receipt of any mRNA vaccine in the 60 days preceding any study intervention administration or planned receipt of any mRNA vaccine in the 60 days following any study intervention administration; (E11) previous vaccination against RSV with an investigational vaccine; (E12) receipt of immune globulins, blood, or blood-derived products in the past 3 months; (E13) receipt of oral or injectable antibiotic therapy within 72 hours prior to the first blood draw; (E14) participation at the time of study enrollment (or in the 4 weeks preceding the first study intervention administration) or planned participation during the present study period in another clinical study investigating a vaccine, drug, medical device, or medical procedure; (E15) deprived of freedom by an administrative or court order, or in an emergency setting, or hospitalized involuntarily; (E16) self-reported or documented human immunodeficiency virus (HIV) detected by any FDA-approved/validated test, hepatitis B virus surface antigen (HbsAg), hepatitis B core antibodies (HbcAb), or hepatitis C virus antibodies (HCV Abs) or positive SARS-CoV-2 RT-PCR or antigen test; or (E17) identified as an Investigator or employee of the Investigator or study center with direct involvement in the proposed study, or identified as an immediate family member (i.e., parent, spouse, natural or adopted child) of the Investigator or employee with direct involvement in the proposed study.

Exclusion criteria, E1-E17, are checked at participant initial screening visit. Additionally, E1-E17 plus three additional exclusion criteria, E18-E20, are checked at the first visit (visit 1; day 1). E18 is a screening electrocardiogram that is consistent with possible myocarditis, pericarditis, and/or myopericarditis or, in the opinion of the investigator, demonstrates clinically relevant abnormalities that may affect participant safety or study results. E19 is moderate or severe acute illness/infection (according to investigator judgment) or febrile illness (temperature 38.0° C.) on the day of study intervention administration. A prospective participant should not be included in the study until the condition has resolved or the febrile event has subsided. E20 is any screening laboratory parameter with laboratory abnormalities that are greater than Grade 1 or deemed clinically significant in the opinion of the Investigator.

If the participant has a primary physician who is not the Investigator, the site should contact this physician with the participant's consent to inform him/her of the participant's participation in the study. In addition, the site should ask this primary physician to verify exclusion criteria relating to previous therapies, such as receipt of blood products or previous vaccines.

### Study Interventions and Concomitant Therapy

### Methods of Randomly Assigning Participants to Intervention Groups

A randomized participant is defined as a participant who has been allocated to a randomized intervention regardless of whether the treatment was administered or not (i.e., participant registered by the IRT). A participant cannot be randomized more than once in the study.

Participants who sign the informed consent form at the screening visit and meet the eligibility criteria at screening visit and at visit 1 will be randomly assigned to one of the study intervention groups at Visit 1, according to the following:
Study A Cohort 1 (Sentinel Cohort 1): RSV mRNA vaccine at low dose with LNP cKK-E10, RSV mRNA vaccine at low dose with LNP GL-HEPES-E3-E12-DS-4-E10, or placebo in a 1:1:1 ratio.
Study A Cohort 2 (Sentinel Cohort 2): RSV mRNA vaccine at medium dose with LNP cKK-E10, RSV mRNA vaccine at medium dose with LNP GL-HEPES-E3-E12-DS-4-E10, or placebo in a 1:1:1 ratio.
Study A Cohort 3 (Sentinel Cohort 3): RSV mRNA vaccine at high dose with LNP cKK-E10, RSV mRNA vaccine at high dose with LNP GL-HEPES-E3-E12-DS-4-E10, or placebo in a 1:1:1 ratio.
Study B (Main Cohort): RSV mRNA vaccine at low dose with LNP cKK-E10, RSV mRNA vaccine at low dose with LNP GL-HEPES-E3-E12-DS-4-E10, RSV mRNA vaccine at medium dose with LNP cKK-E10, RSV mRNA vaccine at medium dose with LNP GL-HEPES-E3-E12-DS-4-E10, RSV mRNA vaccine at high dose with LNP cKK-E10, RSV mRNA vaccine at high dose with LNP GL-HEPES-E3-E12-DS-4-E10, or placebo in a 1:1:1:1:1:1:1 ratio.

At Visit 1, randomization will be stratified by cohort (Sentinel Cohort 1, 2, 3, or Main Cohort), by CMI subset (Yes or No), and by Japanese origin status (Yes or No).

Participants from the placebo group and the selected formulation group (i.e., both the dose-level and the LNP formulation) of Study B (Main Cohort) will be eligible to receive a booster dose 12 months after the first vaccination.

At Visit 8, participants who meet the eligibility criteria, including the booster screening visit (Screening Visit 8), will be randomly assigned in a 1:1 ratio to receive the RSV mRNA vaccine selected formulation or placebo. At Visit 8, randomization will be stratified by the study intervention group randomized at Visit 1.

Site staff will connect to the interactive response technology (IRT), enter the identification and security information, and confirm a minimal amount of data in response to IRT prompts. The IRT will then provide the group assignment and have the site staff confirm it. The IRT will also state whether the participant has been assigned to the CMI subset (i.e., 20 participants of each study intervention group of the Main Cohort, recruited from a limited number of selected sites). If the participant is not eligible to participate in the study, then the information will only be recorded on the participant recruitment log. Participant numbers should not be reassigned for any reason. The randomization codes will be kept securely in the IRT.

### Concomitant Therapy—Reportable Medication

Any medication that the participant received prior to the day of vaccination, is receiving at the time of enrollment, or receives during the study must be reported if the medication affects the interpretation of safety data (e.g., an antipyretic or analgesic that could reduce the intensity or frequency of an adverse event) or may interfere with the development or measurement of an immune response (e.g., the use of immune-suppressors, immune-modulators, or some antibiotics that can impact the effect of certain bioassays) will be reported by the Investigator. Steroid medications can affect both the evaluation of the safety and the immune response to the vaccine.

The following are a list of categories of reportable medications:
Medications impacting or that may have an impact on the evaluation of the safety (e.g., antipyretics, analgesics, and non-steroidal anti-inflammatory drugs (NSAIDs), systemic steroids/corticosteroids).
Note: Topical analgesics should NOT be applied at the injection site of study intervention; however, if they are applied inadvertently, they should be recorded.
Medications impacting or that may have an impact on the immune response (e.g., other vaccines, blood products, antibiotic classes that may interfere with bioassays used by Sanofi Pasteur laboratory or other testing laboratories, systemic steroids/corticosteroids, immune-suppressors, immune-modulators with immunosuppressive properties, anti-proliferative drugs such as DNA synthesis inhibitors.
Medications impacting or that may have an impact on both the safety and the immune response (e.g., systemic steroids/corticosteroids).

Reportable medications will be collected in the case report form (CRF) until the end of the unsolicited follow-up period (i.e., 28 days after vaccination). Medications that may have an impact on the immune response or that may have an impact on both the safety and immune response will be collected throughout the study. mRNA vaccine(s) will be collected throughout the study, including the 28 days after vaccination.

Dosage and administration route, homeopathic medication, topical and inhaled steroids, as well as topical, ophthalmic, and ear treatments will not be recorded (except topical analgesics applied at the injection site of study intervention).

Medications given in response to an adverse event will be captured in the “Action Taken” section of the case report form only. No details will be recorded in the concomitant medication form of the CRF unless the medication(s) received belongs to one of the pre-listed categories. Medications will be coded. Information regarding prior medications, including previous influenza vaccinations and previous mRNA vaccinations/products taken by the participant, will be recorded in the participant's eCRF.

### Rescue Medicine

Appropriate medical equipment and emergency medications, including epinephrine (1:1000), must be available at the study site in the event of an anaphylactic, vasovagal, or other immediate allergic reaction.

### Discontinuation of Study Intervention

Participants will permanently discontinue the study intervention, i.e., will not be eligible to receive the booster dose for should a participant experience at least one of the 13 reasons listed below as definitive contraindications. Additional unscheduled visits may be performed for safety reasons and information will be reported in the source documents.

### Three Temporary Contraindications

Should a participant experience one of the three conditions listed here, the Investigator will postpone further vaccination (i.e., administration of the booster dose for participants eligible to be enrolled in the Booster cohort) until the condition is resolved. Postponement must still be within the timeframe for vaccination indicated in the scheduled activities. (1) Febrile illness (temperature ≥38° C. [≥100.4° F.]) or moderate or severe acute illness/infection on the day of vaccination, according to Investigator judgment. (2) Receipt of any vaccine (other than the study mRNA vaccine) in the 4 weeks preceding the any study vaccination intervention administration or planned receipt of any vaccine other than mRNA vaccine in the 4 weeks following the any study intervention administration. (3) Receipt of any mRNA vaccine in the 60 days preceding any study vaccination intervention administration or planned receipt of any mRNA vaccine in the 60 days following any study intervention administration.

Thirteen Definitive Contraindications for Withdrawal from the Study

Should a participant experience at least one of the conditions listed herein, the Investigator will definitively discontinue vaccination: (1) anaphylaxis or an allergic reaction to the previous dose of vaccine; (2) abnormal laboratory parameter of Grade 2 or 3 and assessed by the Investigator as related to the previous dose of vaccine; (3) SAE assessed as related to the study vaccine following the previous dose of vaccine, based on Investigator's judgment; (4) myocarditis, pericarditis, and/or myopericarditis; (5) RSV-associated illness diagnosed clinically, serologically or microbiologically; (6) thrombocytopenia or bleeding disorder; (7) chronic illness (e.g., cardiac disorders, renal disorders, autoimmune disorder, diabetes, psychiatric disorder, or chronic infection) that, in the opinion of the investigator, is at a stage where it might interfere with study conduct or completion; (8) known or suspected congenital or acquired immunodeficiency; or receipt of immunosuppressive therapy, such as anti-cancer chemotherapy or radiation therapy, within the preceding 6 months; or long-term systemic corticosteroid therapy (prednisone or equivalent for more than 2 consecutive weeks within the past 3 months); (9) receipt of anticoagulants in the 3 weeks preceding booster injection; (10) receipt of immune globulins, blood, or blood-derived products in the past 3 months; (11) receipt of oral or injectable antibiotic therapy within 72 hours prior to the pre-booster vaccination blood draw at visit 7 (BL0005); (12) participation in or plans to participate in another clinical study investigating a vaccine, drug, medical device, or medical procedure during the booster phase of the study; (13) self-reported or documented seropositivity for human immunodeficiency virus (HIV) antigen and/or antibodies, hepatitis B virus surface antigen (hBsAg), hepatitis B core antibodies (hBcAb), or hepatitis C virus antibodies (HCV Abs).

In the event of a local or national immunization program with a pandemic influenza vaccine or any other vaccine as needed, participants who receive that vaccine at any time during the study will not be withdrawn from the study.

A participant may withdraw from the study at any time at his/her own request or may be withdrawn at any time at the discretion of the investigator for safety, behavioral, or compliance reasons. If the participant withdraws consent, the participant will be permanently discontinued both from the study intervention and from the study at that time. Withdrawn participants will not be replaced.

### Study Assessments and Procedures

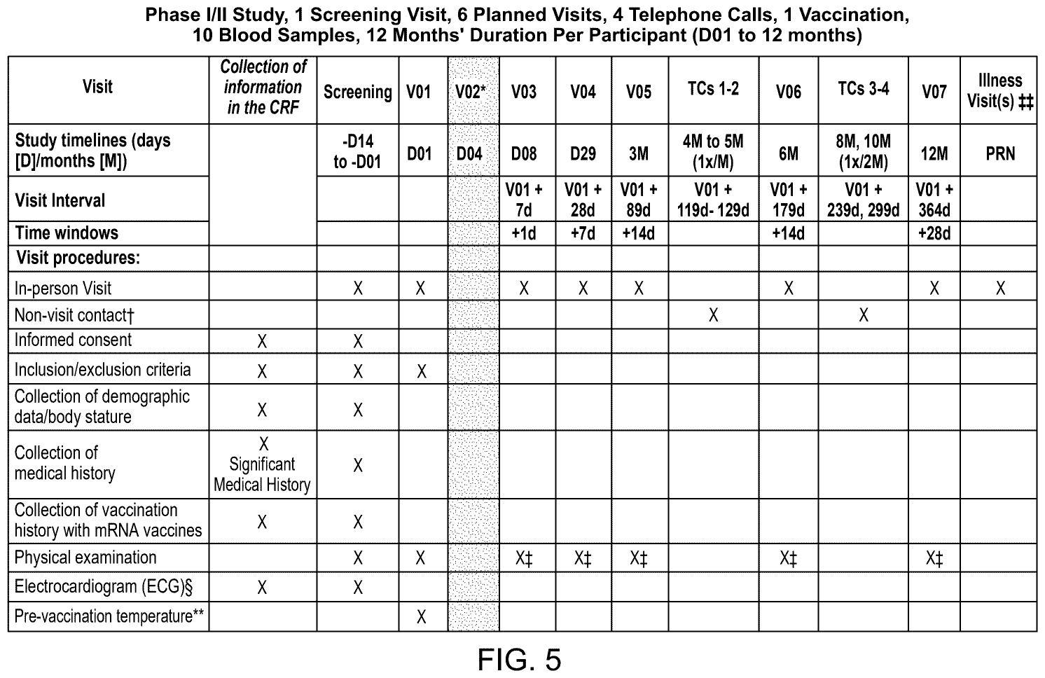

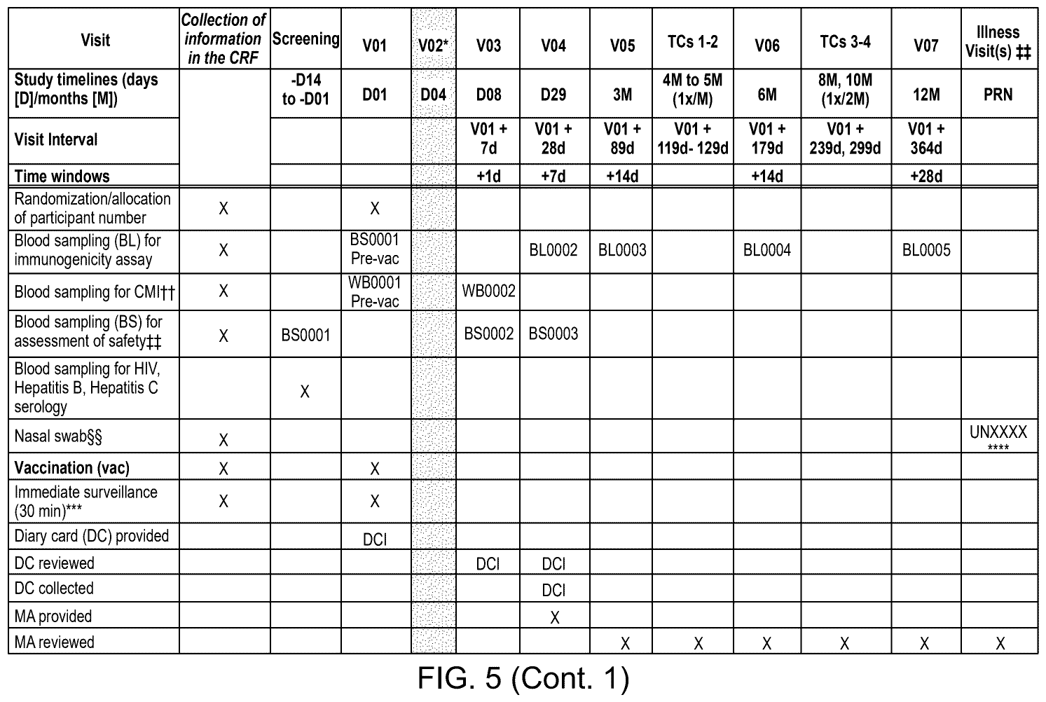

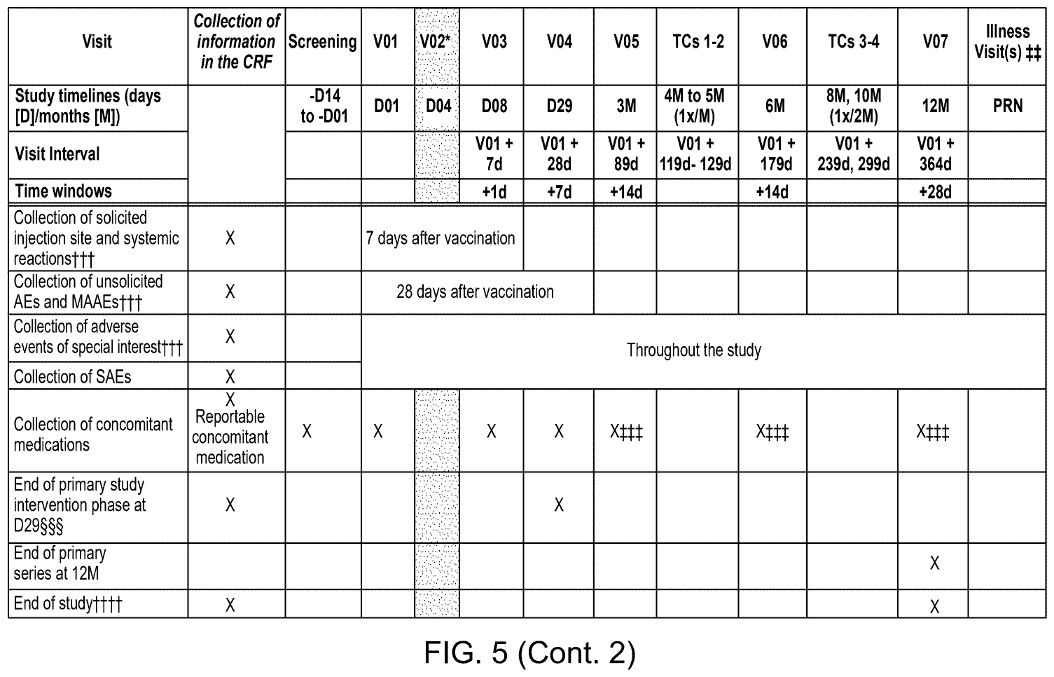

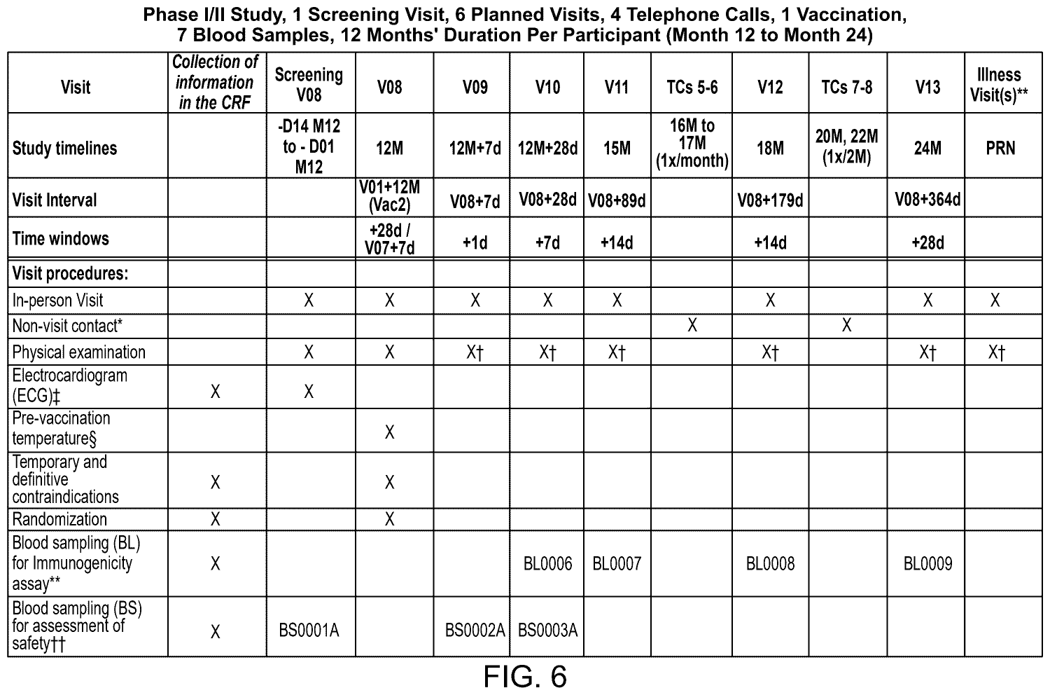

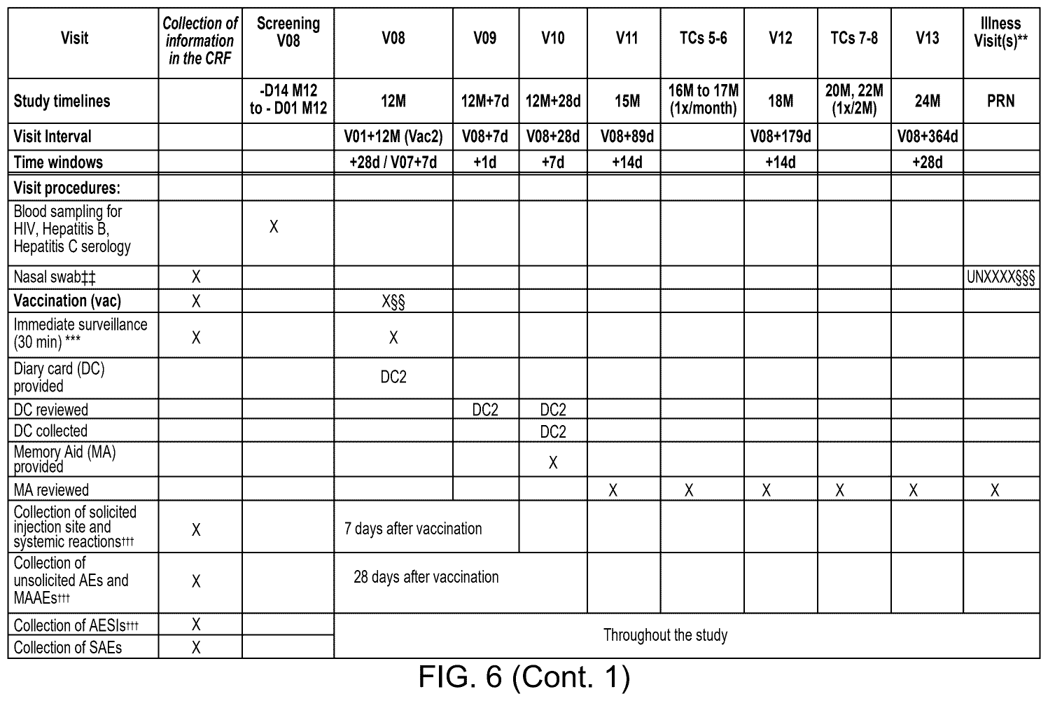

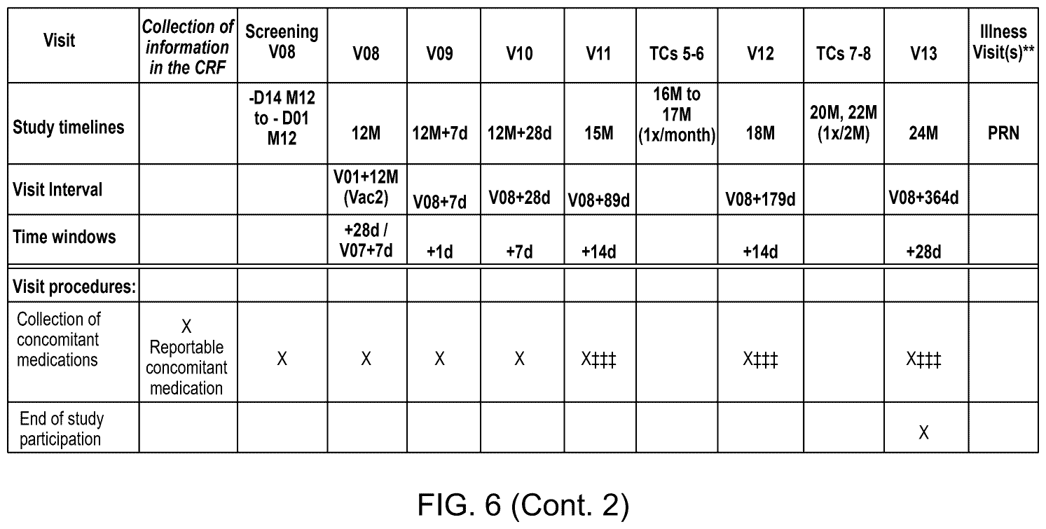

Study assessment data collected at each visit, including routine clinical management (e.g., blood count, electrocardiogram, physical exam) is obtained as depicted in the table for scheduled activities for Study A (Sentinel Cohort) as shown in FIG. 4, for Study B (Main Cohort) as shown in FIG. 5, and Booster Cohort as shown in FIG. 6.

### Blood Samples

Blood samples are collected at visits according to the table of scheduled activities for each cohort as shown in FIG. 4-6, and will be used for the assessment of safety, immunogenicity, and to test serology to HIV, Hepatitis B and C. The maximum amount of blood collected from each participant over the duration of the study, including any extra assessments that may be required, will not exceed 255 mL. The amount of blood collected at each visit will range from 15 mL to 50 mL as shown in Tables 8, 9, and 10 below. Repeat or unscheduled samples may be taken for safety reasons or for technical issues with the samples.

TABLE 8
Blood Sampling volume (mL) per visit - Study A (Sentinel Cohort (participants aged 18 to 50 years))

| | Visit Number (V) | | | | | | | |
|---|---|---|---|---|---|---|---|---|
| | Screening | V01 | V02 | V0 | V04 | V05 | V06 | V07 |
| | Trial Timelines (Days [D]/Months [M]) | | | | | | | |
| | −D 14 to | | | | | | | |
| | D 01 | D 01 | D 04 | D 08 | D 29 | 3 M | 6 M | 12 M |
| | Time Windows (Days) | | | | | | | |
| | | | | | +7 D | +14 D | +14 D | +28 D |
| Screening serology (HIV | X | | | | | | | |
| Ab, hBsAg, hBcAb, HCV | | | | | | | | |
| Ab) (~5 mL) | | | | | | | | |
| Clinical Safety Laboratory | BS0001 | | BS0002 | BS0003 | BS0004 | | | |
| (~15 mL at each timepoint) | | | | | | | | |
| Hematology, Coagulation, | X | | X | X | X | | | |
| Serum Chemistry | | | | | | | | |
| Immunogenicity | | BL0001 | | | BL0002 | BL0003 | BL0004 | BL0005 |
| assessments (20 mL at | | | | | | | | |
| each timepoint) | | | | | | | | |
| RSV Anti-F IgG ELISA | | X | | | X | X | X | X |
| RSV MN | | X | | | X | X | X | X |
| Biobank Sampling | | X | | | | X | X | X |
| (15 mL at each timepoint) | | | | | | | | |
| TOTAL (mL) | 20 | 35 | 15 | 15 | 35 | 35 | 35 | 35 |

BL: blood sample for immunogenicity;
BS: blood sample for safety;
ELISA: enzyme-linked immunosorbent assay;
hBcAb: hepatitis B core antibody;
hBsAg: hepatitis B surface antigen;
HCVAb: hepatitis C virus antibodies;
HIV: human immunodeficiency virus;
IgG: immunoglobulin G;
MN: microneutralization;
RSV: respiratory syncytial virus

TABLE 9
Blood Sampling volume (mL) per visit - Study B (Main Cohort (participants aged 60 years and older))

| | Visit Number (V) | | | | | | | |
|---|---|---|---|---|---|---|---|---|
| | Screening | V01 | V02* | V0 | V04 | V05 | V06 | V07 |
| | Trial Timelines (Days [D]/Months [M]) | | | | | | | |
| | D 14 | | | | | | | |
| | to −D 01 | D 01 | D 04 | D 08 | D 29 | 3 M | 6 M | 12 M |
| | Time Windows (Days) | | | | | | | |
| | | | | +1 D | +7 D | +14 D | +14 D | +28 D |
| Screening serology (HIV | X | | | | | | | |
| Ab, hBsAg, hBcAb, HCV | | | | | | | | |
| Ab) (~5 mL) | | | | | | | | |
| Clinical Safety Laboratory | BS0001 | | | BS0002 | BS0003 | | | |
| (15 mL at each timepoint) | | | | | | | | |
| Hematology, Coagulation, | X | | | X | X | | | |
| Serum Chemistry | | | | | | | | |
| Immunogenicity | | BL0001 | | | BL0002 | BL0003 | BL0004 | BL0005 |
| assessments (20 mL at | | | | | | | | |
| each timepoint) | | | | | | | | |
| RSV Anti-F IgG ELISA | | X | | | X | X | X | X |
| RSV MN | | X | | | X | X | X | X |
| CMI assessments† (4 mL | | WB0001 | | WB0002 | | | | |
| at each timepoint) | | | | | | | | |
| Whole Blood for | | X | | X | | | | |
| TruCulture | | | | | | | | |
| Biobank Samplings | | X | | | X | X | X | X |
| (15 mL at each timepoint) | | | | | | | | |
| TOTAL (mL) | 20 | 39 | — | 19 | 50 | 35 | 35 | 35 |

Abbreviations: BL: blood sample for immunogenicity; BS: blood sample for safety; CMI: cell-mediated immunity; ELISA: enzyme-linked immunosorbent assay; hBcAb: hepatitis B core antibody; hBsAg: hepatitis B surface antigen; HCVAb: hepatitis C virus antibodies; HIV: human immunodeficiency virus; IgG: immunoglobulin G; MN: microneutralization; RSV: respiratory syncytial virus; WB: blood sample for TruCulture.
*Day 3 visit (V02) is not applicable for the Main Cohort
†Only applicable for a subset of participants at some selected sites
_{S} Sample will be collected and stored for potential future safety and immunological research

TABLE 10
Blood Sampling volume (mL) per visit - Study C (Booster Cohort (participants aged 60 years and older))

| | Visit Number (V) | | | | | | |
|---|---|---|---|---|---|---|---|
| | Screening | | | | | | |
| | V08 | V08 | V09 | V10 | V11 | V12 | V13 |
| | Trial Timelines (Days [D]/Months [M]) | | | | | | |
| | −D 14 | | | | | | |
| | M 12 | | | | | | |
| | to −D 01 | 12 M | V08 + 7 D | | | V08 + 179 D | V08 + 364 D |
| | M 12 | +7 D | +1 D | V08 + 28 D | V08 + 89 D | +14 D | +28 D |
| Screening serology (HIV | X | | | | | | |
| Ab, hBsAg, hBcAb, HCV | | | | | | | |
| Ab) (~5 mL) | | | | | | | |
| Clinical Safety Laboratory | BS0001A | | BS0002A | BS0003A | | | |
| (15 mL at each timepoint) | | | | | | | |
| Hematology, Coagulation, | X | | X | | | | |
| Serum Chemistry | | | | | | | |
| Immunogenicity | | | | BL0006 | BL0007 | BL0008 | BL0009 |
| assessments (20 mL | | | | | | | |
| at each timepoint) | | | | | | | |
| RSV Anti-F IgG ELISA | | | | X | X | X | X |
| RSV MN | | | | X | X | X | X |
| Biobank Sampling | | | | X | X | X | X |
| (15 mL at each timepoint)* | | | | | | | |
| TOTAL (mL) | 20 | 0 | 15 | 50 | 30 | 30 | 30 |

Abbreviations: BL: blood sample for immunogenicity;
BS: blood sample for safety;
ELISA: enzyme-linked immunosorbent assay;
hBcAb: hepatitis B core antibody;
hBsAg: hepatitis B surface antigen;
HCVAb: hepatitis C virus antibodies;
HIV: human immunodeficiency virus;
IgG: immunoglobulin G;
MN: microneutralization;
RSV: respiratory syncytial virus
*Sample will be collected and stored for potential future safety and immunological research

### Immunogenicity Assessments

### RSV Anti-F IgG ELISA

Antibodies to the RSV-F antigen are measured using the RSV Anti-F IgG ELISA. Briefly, RSV-F antigen is coated onto a microtiter plate and serial 2-fold dilutions of human serum samples are added and incubated to allow binding to the RSV-F antigen. A horseradish peroxidase (HRP)-conjugated anti-human IgG detection antibody is then added and followed by colorimetric substrate. The concentration of IgG antibodies to RSV-F antigen is calculated over 6-serial fold dilutions relative to qualified internal reference calibrated against the WHO International standard (1^{st} International Standard for antiserum for RSV) with an assigned value (International Units/mL).

### RSV Neutralizing Antibody Assessment

RSV neutralizing antibodies are measured using a microneutralization (MN) assay, Nexelis PRNT A2 assay or A Long assay. Serial, two-fold dilutions of sera samples are heat-inactivated and then mixed with a constant concentration of the RSV A2 strain (ATCC VR-1540). The mixtures are inoculated into wells of a 96-well microplate with permissive hEp-2 cells (ATCC CCL-23) and incubated for 2 days. A reduction in virus infectivity (viral antigen production) due to neutralization by antibody present in serum samples is detected by ELISA. After washing and fixation, RSV antigen production in cells is detected by successive incubations with a mouse anti-RSV-specific monoclonal antibody, HRP-anti-mouse IgG conjugate, and a chromogenic substrate. The resulting optical density is measured using a microplate reader. The reduction in RSV infectivity as compared to that in the virus control wells constitutes a positive neutralization reaction indicating the presence of neutralizing antibodies in the serum sample.

### Cell-Mediated Immunity

T helper cell response is assessed by using fresh whole blood (TruCulture). The T cell analysis in the whole blood, collected, using RBM TruCulture whole blood collection and culture system, enables consistent, reliable assessment of the T helper cells polarization. The Triculture tube containing the stimulant or antigen of choice, allows almost instantaneous stimulation of the cells in the presence of all the blood components, after the blood is drawn into the tube and therefore, minimizes variability that may arise due to the handling and manipulation of blood, including processing for peripheral blood mononuclear cells (PBMC). Preliminary laboratory results and previous reports in the literature have shown the robustness of the method. Duffy et al. (2017), Clin Immunol, 183:325-335. The assay relies on the fact that the T helper cytokines are secreted in a single tubes post stimulation and supernatant are collected at 24 or 48 hours. A panel of cytokine using the Luminex xMAP technology from RBM TruCulture, is measured to determine the status of the T helper cell polarization.

### Safety Assessments

Planned time points for all safety assessments are provided in the scheduled activities tables for each cohort as shown in FIG. 4-6. Prior to enrollment, participants are assessed for pre-existing conditions and illnesses, both past and ongoing and such conditions are documented. Significant (clinically relevant) medical history (reported as diagnosis) including conditions/illnesses for which the participant is or has been followed by a physician or conditions/illnesses that could resume during the study or lead to an SAE or to a repetitive outpatient care is reported in the case report form. In addition, history of receipt of mRNA-based vaccines will be recorded.

### Physical Examinations and Vital Signs

At the Screening visit, the Investigator or a designee will perform a full physical examination. A full physical examination will also be completed at Visit 1. Targeted/abbreviated physical exams will also be completed for all subsequent in-person visits at timepoints specified in the scheduled activities tables for each cohort as shown in FIG. 4-6. Oral or axillary pre-vaccination temperature will be systematically collected by the investigator. Tympanic, skin, and temporal artery thermometers must not be used.

### Electrocardiograms

An ECG is performed at the screening visit to serve as a baseline and to exclude participants with probable or possible myocarditis, pericarditis, and/or myopericarditis, as well as to identify participants with clinically relevant abnormalities that may affect participant safety or study results. In the event a participant develops symptoms of myocarditis, pericarditis, and/or myopericarditis during the conduct of the study, additional ECG(s) will be performed as rapidly as possible, at an unscheduled visit, if necessary. The ECG will be recorded, and any assessment thereof will be based on standard medical care.

### Clinical Safety Laboratory Assessments

Table 11 below lists the clinical laboratory tests. The laboratory tests are performed during the timepoints specified in the scheduled activities tables for each cohort as shown in FIG. 4-6. The investigator reviews the laboratory report and record any clinically significant changes occurring after either the primary or the booster vaccination as an adverse event.

All laboratory tests with values considered clinically significantly abnormal after either the primary or the booster vaccination are repeated until the values return to normal or baseline or are no longer considered clinically significant by the investigator. If clinically significant/any values do not return to normal/baseline within a period of time judged reasonable by the investigator, the etiology should be identified, and the Sponsor notified.

TABLE 11
Clinical safety laboratory tests

| Laboratory | Time period for | |
|---|---|---|
| assessments | assessment | Parameters |
| Hematology | Screening | White blood cell (WBC) count with differential: |
| | V02 (D 04; Sentinel Cohort | Neutrophils |
| | only) | Lymphocytes |
| | V03 (D 08) | Eosinophils |
| | V09 (7 d after booster | Hemoglobin |
| | vaccination; Booster | Platelet count |
| | Cohort only) | Red blood cell (RBC) count |
| Clinical Chemistry | Screening | Blood urea nitrogen (BUN) |
| | V02 (D 04; Sentinel Cohort | Creatinine |
| | only) | Glucose (random) |
| | V03 (D 08) | Potassium |
| | V04 (D 29; Main Cohort | Sodium |
| | Only) | Calcium |
| | V09 (7 d after booster | Aspartate Aminotransferase (AST) |
| | vaccination; Booster | Alanine Aminotransferase (ALT) |
| | Cohort only) | Alkaline phosphatase |
| | | Total bilirubin |
| | | Direct bilirubin |
| | | Total protein |
| | | Troponin I |
| Coagulation | Screening | Prothrombin time |
| parameters | V02 (D 04; Sentinel Cohort | Activated partial thromboplastin time (APTT) |
| | only) | |
| | V03 (D 08) | |
| | V09 (7 d after booster | |
| | vaccination; Booster | |
| | Cohort only) | |
| Other Screening | Screening | HIV antigen/antibody test |
| Tests | | Hepatitis B surface antigen |
| | | Hepatitis B core antibody |
| | | Hepatitis C antibody |
| | | SARS-CoV-2 RT-PCR or antigen test |
| | | Urine or serum pregnancy test (V01) |

### Nasal Swab Collection

Nasal swab specimens for the detection of RSV and respiratory pathogens (including COVID-19) are collected from participants with any respiratory disease episodes. If a participant visits any other non-study doctor/hospital at any time in the study, a nasal swab sample will be obtained at the study site once the subject is discharged, if deemed appropriate by the investigator. All the nasal swab specimens will be collected in the recommended viral transport media tube and will be stored at −60° C. to −80° C. until ready to ship. The requirement for an at-home or on-site illness visit will be evaluated first by video call to enable remote evaluation of severity and remote management of mild (Grade 1) illness, as deemed appropriate by the study investigator.

### Lower Respiratory Tract and Acute Respiratory Disease Assessments

The following RSV disease categories will be used in cases where RSV confirmed by RT-PCR: (1) RSV acute respiratory disease (ARD) is any respiratory symptoms including nasal congestion, sore throat, hoarseness, new or worsening cough, sputum production and dyspnea with or without fever; (2) Severe RSV ARD is an acute respiratory disease with history of fever or measured fever of ≥38° C. and cough with onset within the last 10 days and requires hospitalization, confirmed by RT-PCR; (3) non medically-attended RSV lower respiratory tract disease (LRTD) is an ARD with one or more symptoms of lower respiratory tract illness (this includes affection of the lower respiratory tract: trachea, bronchi and lungs, could be combined i.e., bronchopneumonia, tracheobronchitis) with 10 days of ARD symptom onset and RSV confirmed by RT-PCR; (4) RSV LRTD medically attended is ARD with one or more symptoms of lower respiratory tract illness (this includes affection of the lower respiratory tract: trachea, bronchi and lungs, could be combined, i.e., bronchopneumonia, tracheobronchitis) with 10 days of ARD symptom onset and confirmed by RT-PCR, seeking medical attention. Medical attention is classified as emergency room, hospitalization, outpatient clinic visit.

### Example 2: Interim Analysis 2—Titers and Safety Data

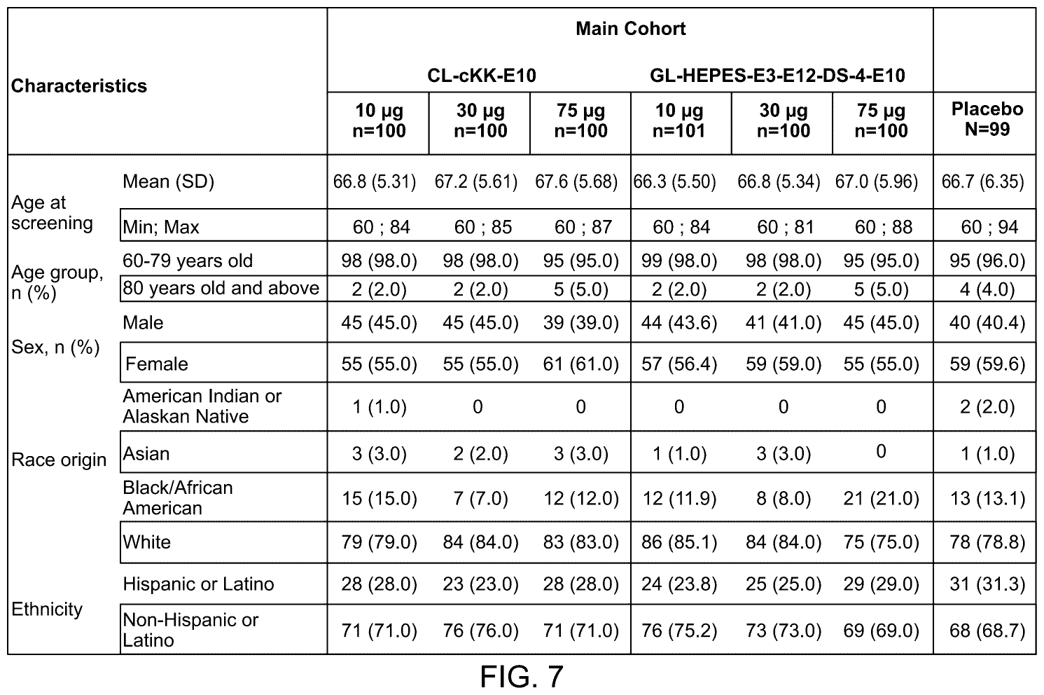

The planned sample size (IA2) was 790, with 90 sentinel cohorts and 700 main cohorts. The IA2 sample size (Partial Main Cohort) was 667 participants to analyze immunogenicity (DO and D29), and 698 participants to analyze safety data (through day 29 (D29)). The demographic characteristics of the Main Cohort is set forth at FIG. 7.

### Titers

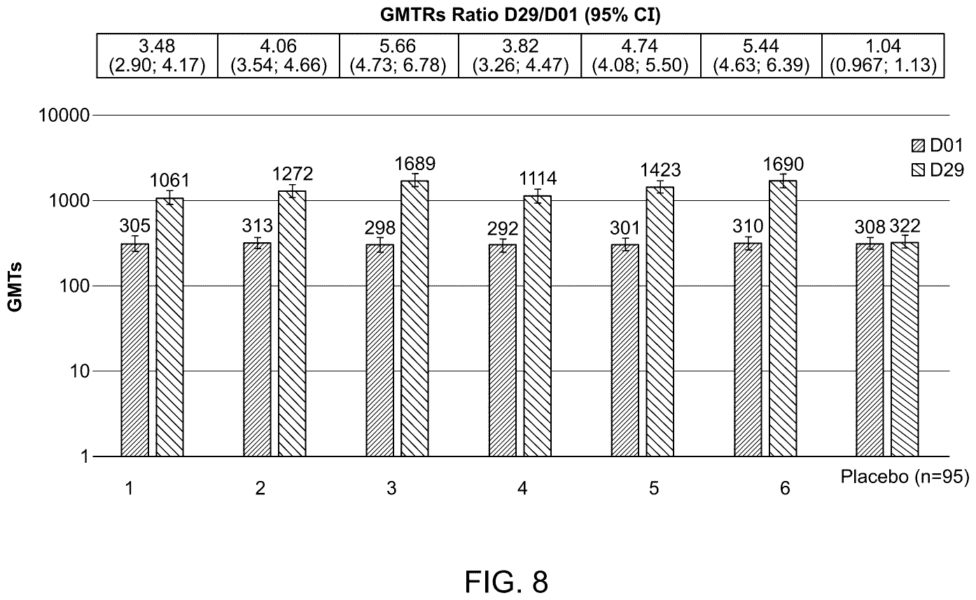

Results of the full Main Cohort (60 years and above) summary of geometric mean titers (GMTs) of RSV A neutralizing antibody (nAb) titers after primary vaccination and neutralizing antibody geometric mean titer ratios (GMTRs) D29/D01 (PPAS-1) are shown at FIG. 8. The cKK-E10+RSV mRNA 75 mcg group showed slightly higher GMTs at D29 with a GMTR of 5.66 compared to the GL-HEPES-E3-E12-DS-4-E10+RSV mRNA 75 mcg group (GMTR of 5.44). These were followed by the GL-HEPES-E3-E12-DS-4-E10+RSV mRNA 30 mcg group with a GMTR of 4.74.

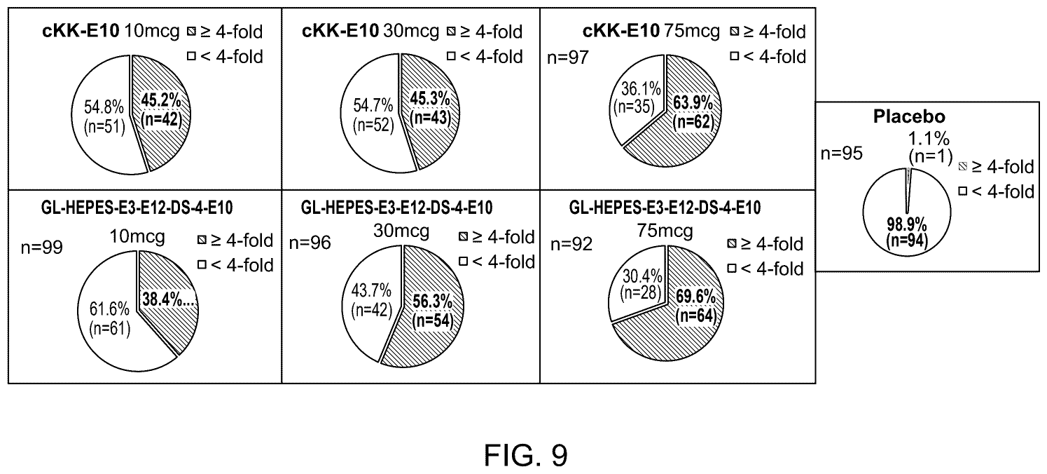

The fold rise data for RSV-A neutralizing antibody after primary vaccination for the Full Main Cohort is shown at FIG. 9. The percentage of participants having at least a 4-fold rise ranged from 38.4% to 69.6% depending upon the dose and LNP.

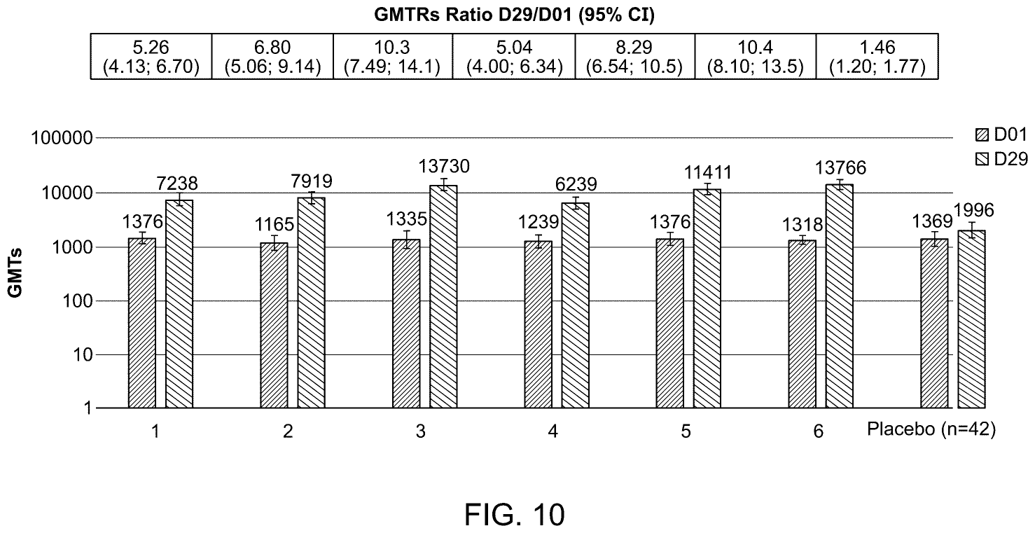

Results of partial Main Cohort (60 years and above) summary of geometric means of IgG antibody titers after primary vaccination and IgG antibody GMTRs are set forth at FIG. 10. The GL-HEPES-E3-E12-DS-4-E10+75 mcg group showed higher IgG GMs at D29 with the highest-fold rise (10.4), followed by the cKK-E10+75 mcg group (10.3) and the GL-HEPES-E3-E12-DS-4-E10+30 mcg group (8.29). These data correlated with the groups showing the best RSV-A GMTs responses.

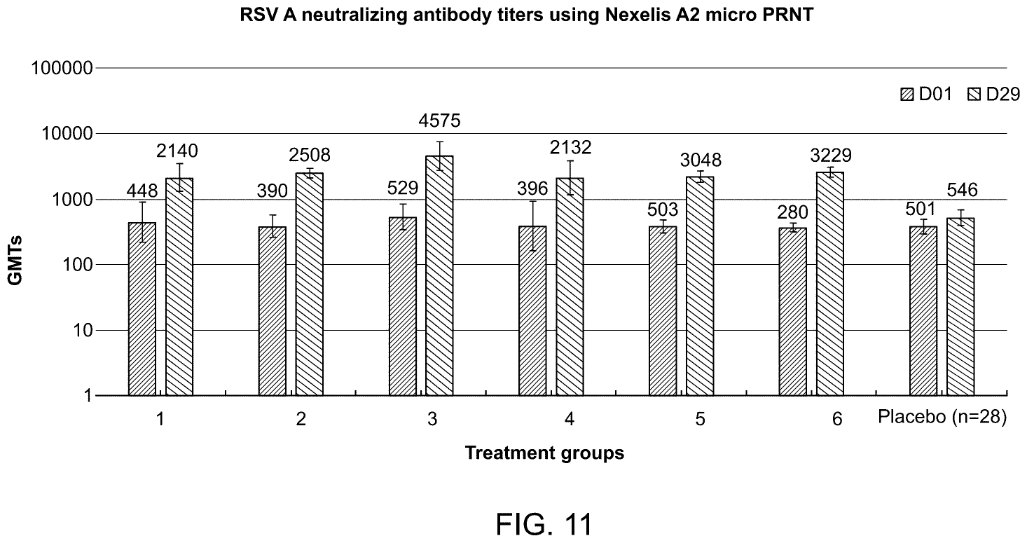

GMT results for the sentinel cohorts (18-50 years) of RSV A neutralizing antibody titers after primary vaccination (PPAS-1) and neutralizing antibodies geometric mean titer ratios (GMTRs) D29/D01 are summarized at FIG. 11. In the sentinel cohorts, the GL-HEPES-E3-E12-DS-4-E10+75 mcg group showed the higher GMTs at D29 with a GMTR of 11.5, followed by the cKK-E10+75 mcg group with a GMTR of 8.64 and, the cKK-E10 and GL-HEPES-E3-E12-DS-4-E10+30 mcg groups with GMTRs of 6.43 and 6.06, respectively.

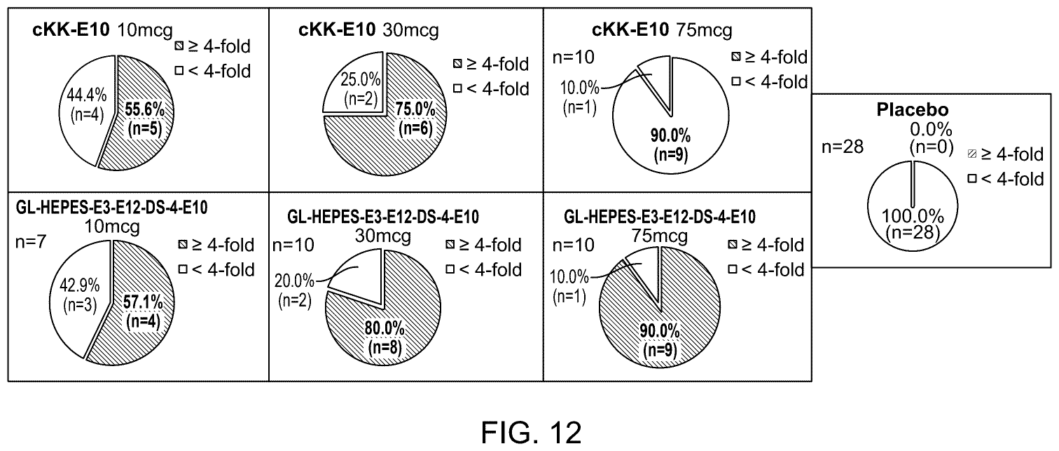

The fold rise data for RSV-A neutralizing antibody after primary vaccination for Sentinel Cohorts is shown at FIG. 12. The percentage of participants having at least a 4-fold rise ranged from 55.6% to 90.0% depending upon the dose and LNP.

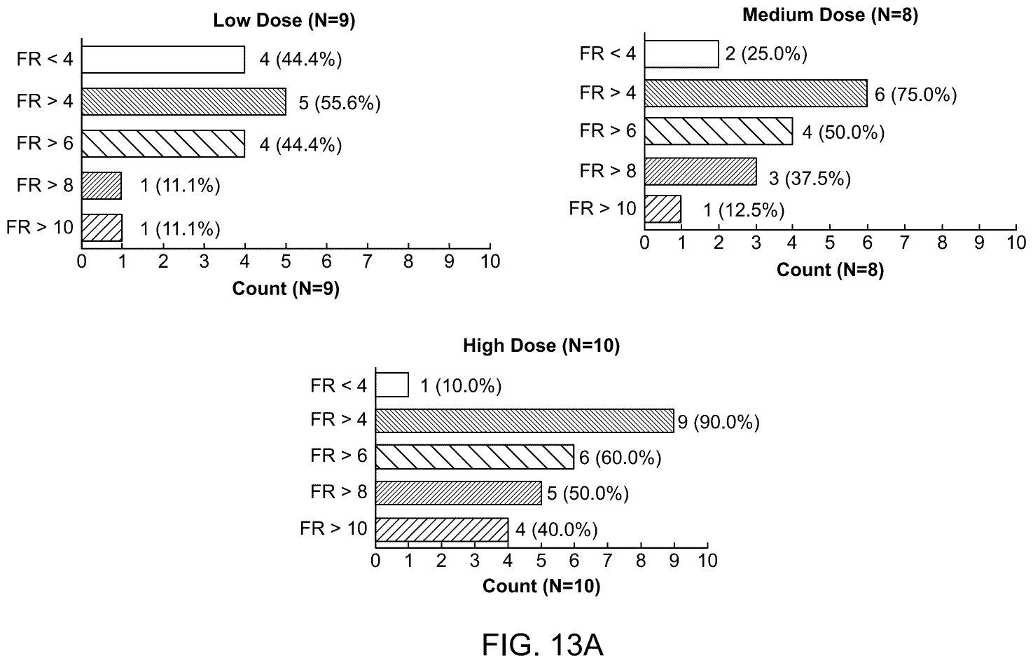

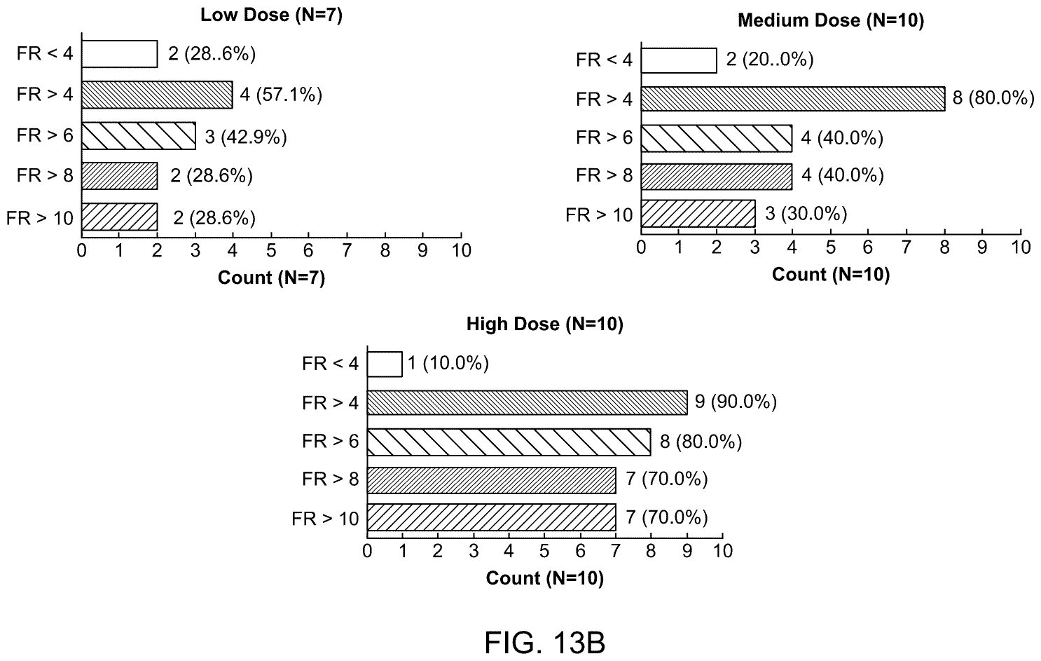

A summary of Sentinel Cohorts for RSV-A neutralizing antibody titers after primary vaccination is shown at FIGS. 13A-13B.

### Conclusions Regarding Immunogenicity

The GL-HEPES-E3-E12-DS-4-E10+RSV mRNA 75 mcg group showed higher GMTs at D29 with a GMTR of 5.22, followed by the cKK-E10+RSV mRNA 75 mcg group, with a GMTR of 4.56 and the GL-HEPES-E3-E12-DS-4-E10+RSV mRNA 30 mcg group with a GMTR of 4.4.

IgG results correlated with the RSV-A neutralizing antibodies responses (higher titers and fold-rise in the same groups).

Higher doses of mRNA (75 mcg) showed higher immunogenicity.

GL-HEPES-E3-E12-DS-4-E10 was associated with higher immunogenicity (i.e., GL-HEPES-E3-E12-DS-4-E10+RSV mRNA 75 mcg and GL-HEPES-E3-E12-DS-4-E10+RSV mRNA 30 mcg groups).

### Safety

Safety data in older adults showed that all RSV mRNA formulations were generally well tolerated.

GL-HEPES-E3-E12-DS-4-E10 groups showed a trend of better reactogenicity compared to cKK-E10, in particular, injection site pain and myalgia (the most frequently reported solicited reaction). Solicited reactions were generally mild to moderate, with few grade 3 reactions. A dose response was observed across mRNA groups.

Unsolicited adverse effects (AEs) were overall balanced among mRNA groups (with trend of slightly more reported in mRNA groups compared to placebo). No dose response was observed.

An increase from baseline of a cardiac biomarker (troponin I level) at Day 8 was observed for five participants (baseline normal), all in mRNA groups (this observation considered as not meaningful at this stage). One high troponin level was reported as a related serious adverse effect (SAE) (Asymptomatic myocardial injury; adjudicated by Cardiac Adjudication Committee). Strenuous exercise was determined as a potential cause of elevated troponin.

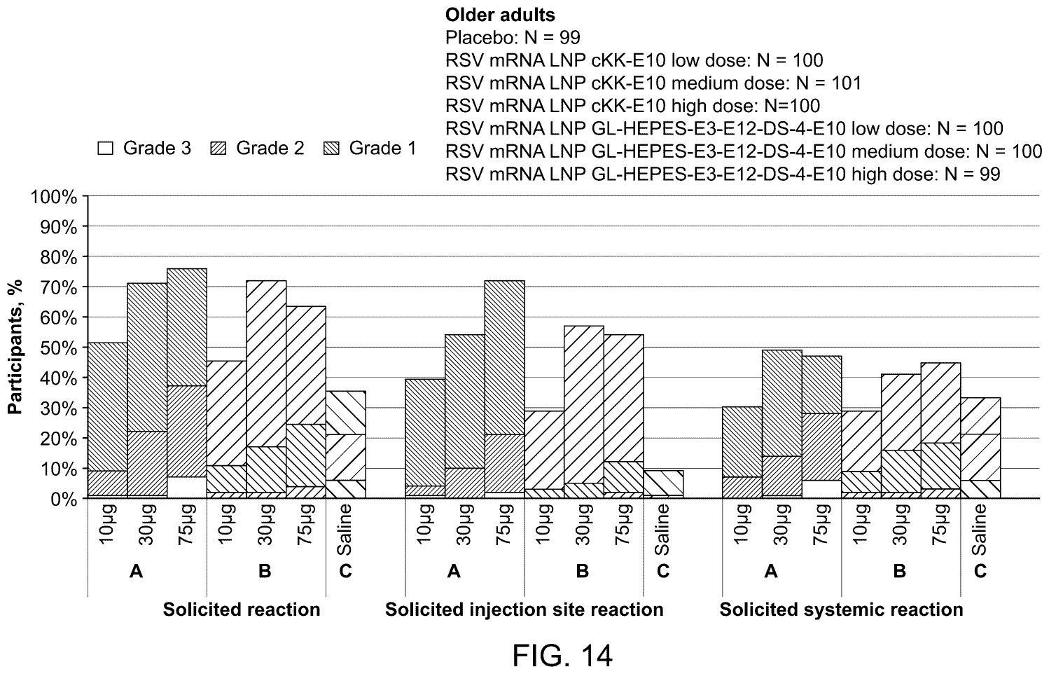

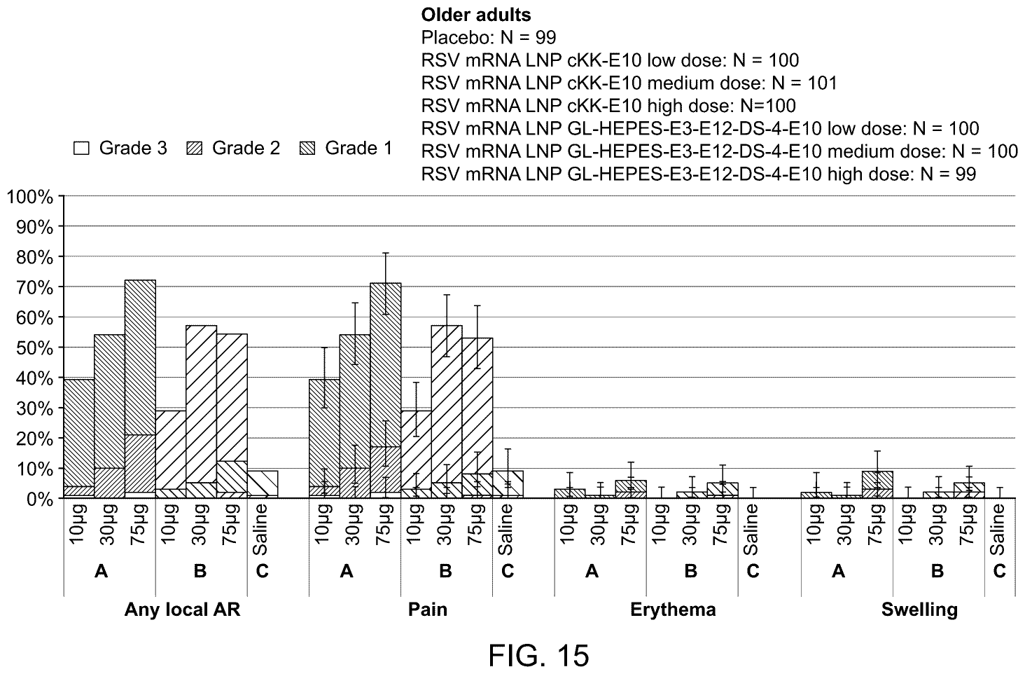

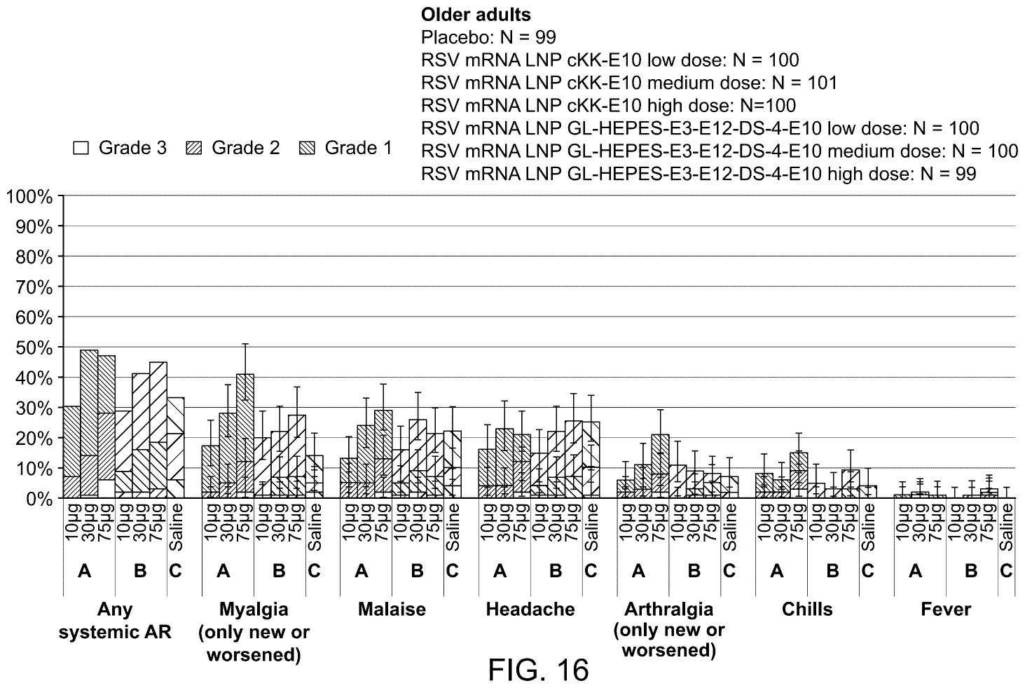

A summary of solicited reactions within 7 days after primary vaccination (%) is shown at FIG. 14. Most solicited reactions were mild-to-moderate and short in duration. A summary of injection site reactions within 7 days after primary vaccination (%) is shown at FIG. 15. Most injection site reactions were mild-to-moderate and short in duration. A summary of solicited systemic reactions within 7 days after primary vaccination (%) is shown at FIG. 16. Most solicited systemic reactions were mild-to-moderate and short in duration.

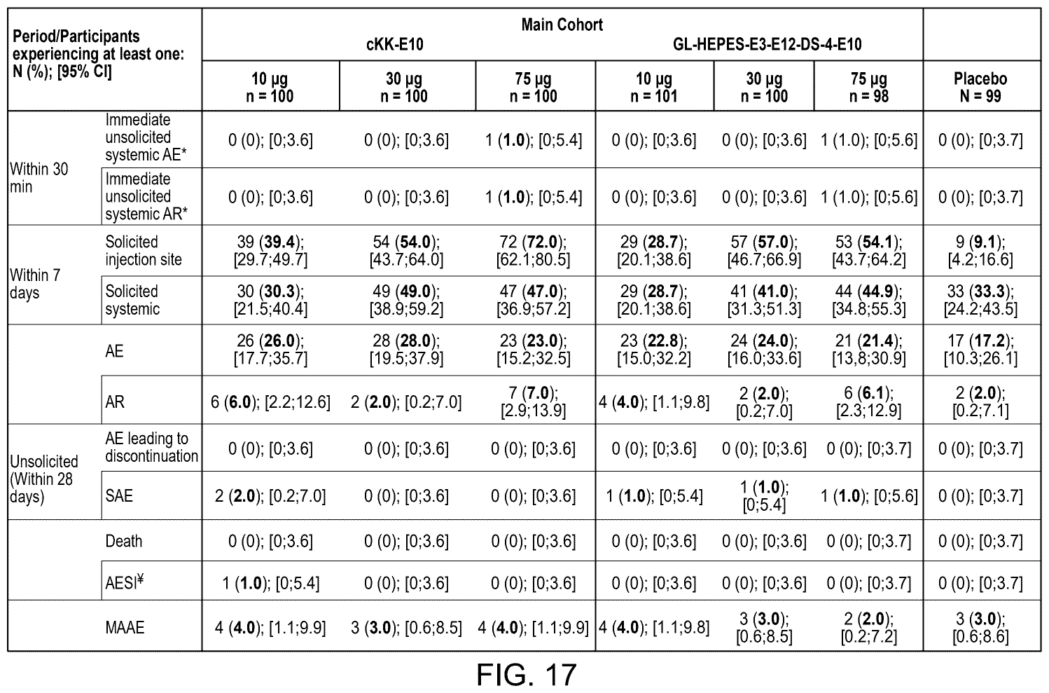

A safety overview after primary vaccination is depicted at FIG. 17.

### Reactogenicity Overview

Overall, injection site pain was the most frequently reported solicited injection site reaction in the 6 mRNA arms (39.4% to 71.0% for the cKK-E10 groups. 28.7% to 53.1% for the GL-HEPES-E3-E12-DS-4-E10 groups).

Myalgia, malaise, and headache were the most frequently reported solicited systemic reaction in the 6 mRNA arms. Myalgia (ranged from 17.2% to 41.0% for the cKK-E10 groups), malaise (13.1 to 29.0% for the cKK-E10 groups) and headache (16.2 to 23.0% for the cKK-E10 groups, with 23% in the medium dose) were the most frequently reported solicited systemic reaction in the cKK-E10 groups, closely followed by arthralgia and chills at the highest dose (21.0% and 15.0%, respectively). The same trend was observed for the GL-HEPES-E3-E12-DS-4-E10 groups (myalgia ranged from 19.8 to 27.6%; malaise from 15.8 to 26.0%, with 26.0% in the medium dose; and headache from 14.9 to 25.5%), except for arthralgia and chills which, tended to be reported as in the placebo groups (even for the highest dose, 8.2% and 9.2%, respectively).

Most of the solicited reactions started between D01-D04, and most of were short in duration (lasted 1-3 days). Solicited reactions were generally mild-to-moderate, with few grade 3 reactions (<4.1% in the GL-HEPES-E3-E12-DS-4-E10 groups and <7.0% in the cKK-E10 groups) and all grade 3 lasted 1 to 3 days at maximum intensity (except one outliner with injection site erythema lasting 5 days at maximum intensity).

A dose response was observed across mRNA groups, with a trend of better reactogenicity in the GL-HEPES-E3-E12-DS-4-E10 groups compared to cKK-E10 groups (GL-HEPES-E3-E12-DS-4-E10 high dose≈cKK-E10 medium dose).

No delayed injection site reactions were observed.

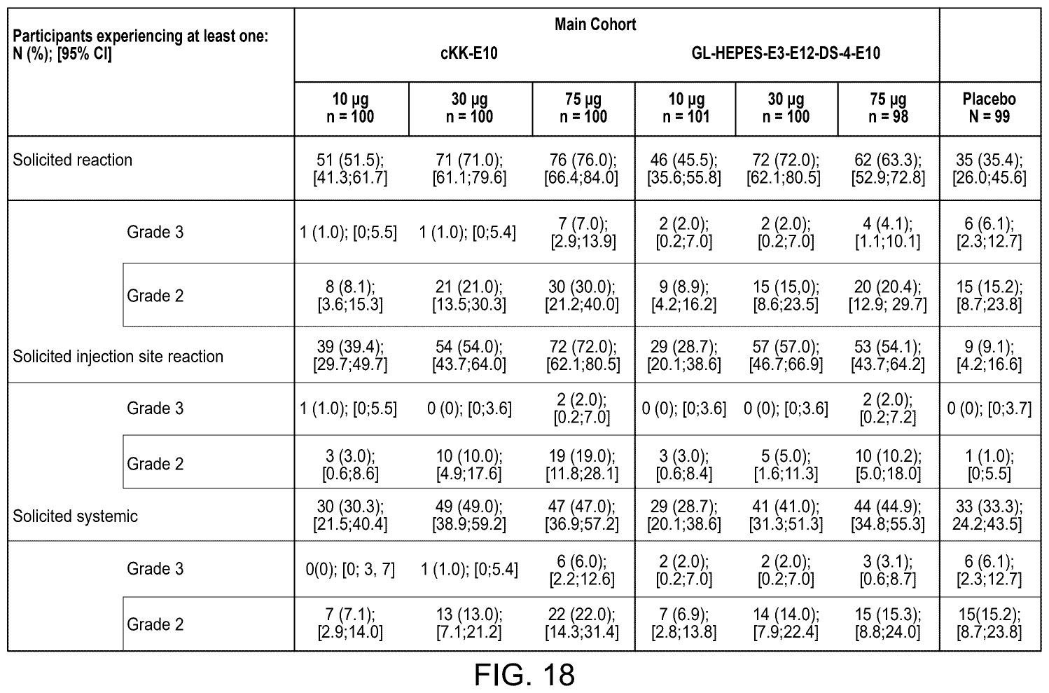

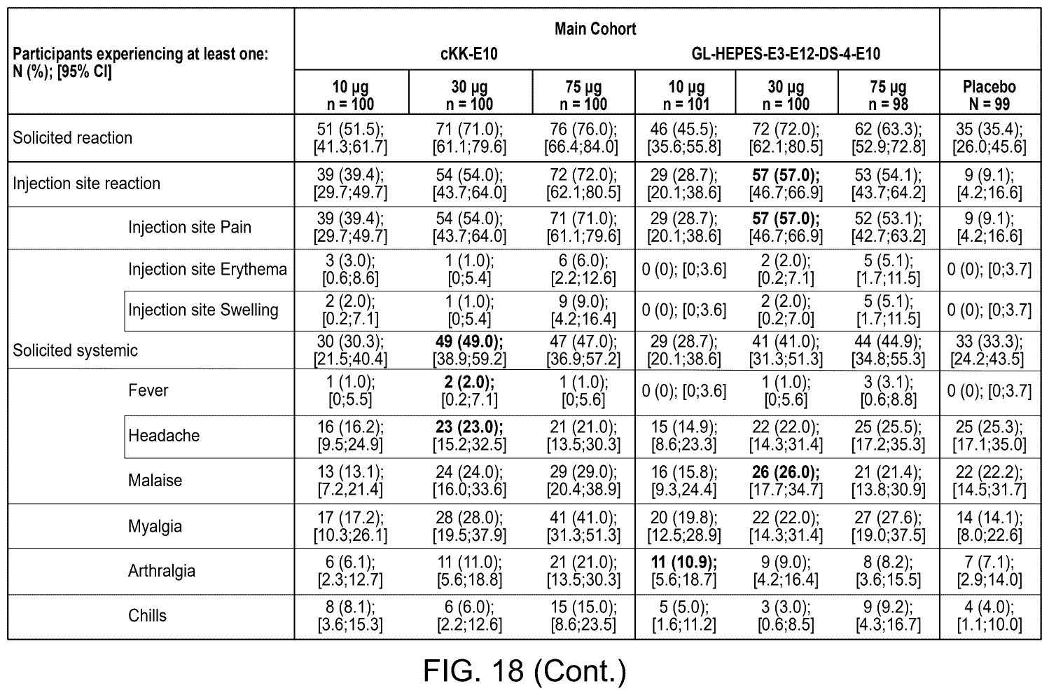

A summary of solicited reactions within 7 days after primary vaccination is shown at FIG. 18.

### Unsolicited Adverse Effects (AEs)

Two subjects reported immediate unsolicited AE/adverse reaction (AR), both in mRNA high doses groups. Both reported hypertension (SOC: vascular disorder) assessed as related to IMP (both grade 1).

Unsolicited AEs tended to be slightly more reported in mRNA groups compared to placebo, and among mRNA groups, an overall balance was observed between mRNA groups: 25.7% subjects reported at least 1 AE in pooled cKK-E10, 22.7% in pooled GL-HEPES-E3-E12-DS-4-E10, and 17.2% in placebo. No dose response was observed. An observed slight imbalance was considered as not meaningful and driven by musculoskeletal and connective tissue disorders (such as arthralgia, myalgia, muscle spasms), and gastrointestinal disorders (such as abdominal pain, diarrhea, nausea). There were few unsolicited ARs (AE assessed as related to IMP): 5% of subjects in pooled cKK-E10; 4% in pooled GL-HEPES-E3-E12-DS-4-E10; and 2% in placebo.

23 subjects had medically attended adverse events (MAAEs). MAAEs were balanced among groups.

Five subjects experienced serious adverse events (SAEs) within 28 days. All were in mRNA groups, equally distributed across arms (2 in LNP cKK-E10 and 3 in LNP GL-HEPES-E3-E12-DS-4-E10). Only one was assessed as related to the investigational medical product IMP (mRNA LNP cKK-E10 low dose) (asymptomatic myocardial injury).

No adverse events of special interest (AESIs) (i.e., anaphylactic reactions (including bronchospasms, and laryngeal spasms), myocarditis, pericarditis, and myopericarditis) were observed. An AESI in the biostatistical tables was downgraded as a non-AESI by the investigator after the cut-off date (diagnosis changed from myocarditis to asymptomatic myocardial injury). No AEs leading to study discontinuation and no deaths were reported.

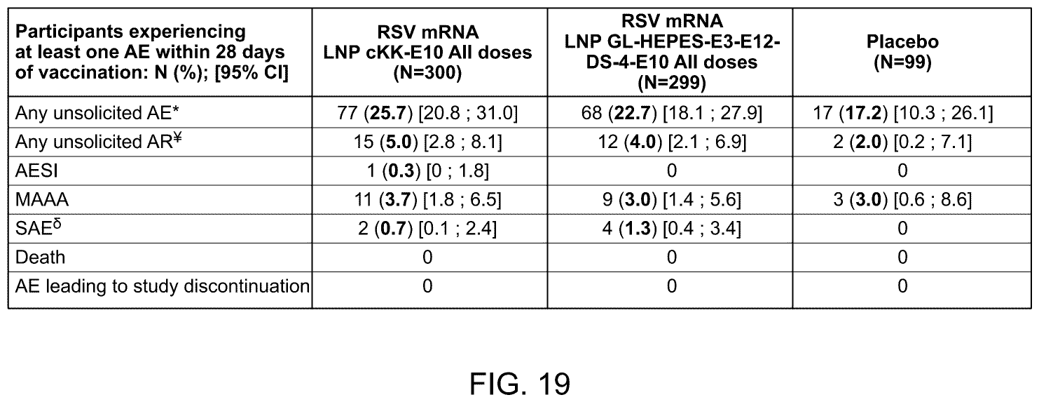

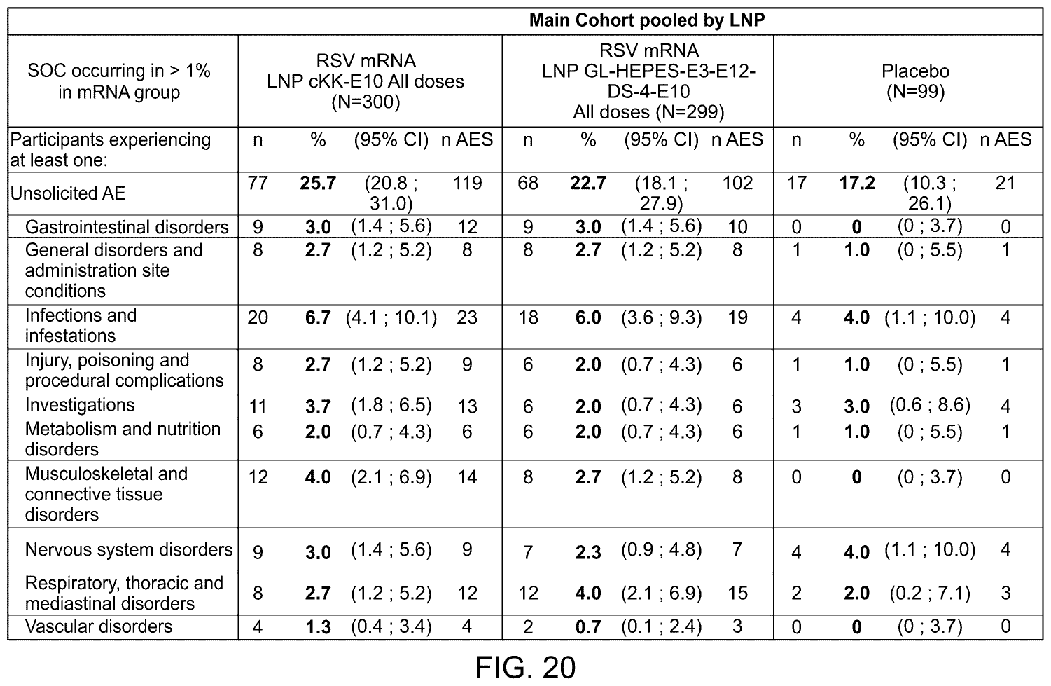

A summary of unsolicited AEs is set forth at FIG. 19. A summary of unsolicited AEs due to gastrointestinal disorders, musculoskeletal disorders and connective tissue disorders is set forth at FIG. 20.

### Overall Conclusions

The GL-HEPES-E3-E12-DS-4-E10+RSV mRNA 75 mcg group showed higher GMTs at D29 with a GMTR of 5.22, followed by the cKK-E10+RSV mRNA 75 mcg group with a GMTR of 4.56 and the GL-HEPES-E3-E12-DS-4-E10+RSV mRNA 30 mcg group with a GMTR of 4.4

Higher doses of mRNA (75 mcg) showed higher immunogenicity.

GL-HEPES-E3-E12-DS-4-E10 was associated with higher immunogenicity (i.e., GL-HEPES-E3-E12-DS-4-E10+RSV mRNA 75 mcg and GL-HEPES-E3-E12-DS-4-E10+RSV mRNA 30 mcg groups). GL-HEPES-E3-E12-DS-4-E10 groups showed a better reactogenicity trend compared to cKK-E10 (GL-HEPES-E3-E12-DS-4-E10 high dose≈cKK-E10 medium dose).

GL-HEPES-E3-E12-DS-4-E10+mRNA 30 mcg is the best for safety overall across Grade 3 and Grade 2 solicited events and special events.

### Example 3: Phase IIb/III—Stage II

### XII. Study Rationale

RSV is the leading viral agent causing severe respiratory tract disease worldwide in older adults. There is a medical need to improve patient quality of life by preventing lower respiratory tract disease (LRTD). The clinical trials described in Example 1, the Study A (i.e., Sentinel Cohort) and Study B (i.e., Main Cohort) will include a subsequent Stage 2, Phase IIb/III study that will enroll approximately 13,482 adults aged 60 years and older to assess the efficacy, immunogenicity, and safety of the LNP selected in Stage 1 (i.e., Phase/IIIa as described in Examples 1 and 2), with a 110 μg dose of RSV mRNA vaccine for the prevention of LRTD due to RSV.

During Stage 1, the 75 μg dose of RSV mRNA vaccine combined with the selected LNP showed the highest immune response with a favorable safety profile. Accordingly, Example 3 will test the 110 μg dose of RSV mRNA vaccine with the selected LNP. The Phase IIb/III of this study is designed to assess the safety of the RSV mRNA vaccine 110 μg dose with the selected LNP and primarily demonstrate the clinical efficacy of the selected RSV mRNA vaccine candidate for the prevention of RSV-LRTD.

### A. Study Overview and Study Design

### Number of Participants, Intervention Groups Overview, and Duration

Number of Participants. A total of 13,482 participants are planned to be randomized, with 50 participants (25 participants per arm) in the Phase IIb Sentinel Cohort and 5190 participants (2595 participants per arm) in the Phase IIb Main Cohort as shown in Table 12 below.

TABLE 12
Experimental Sample Size for Phase IIb/III
(participants aged 60 years or older)

| | | | Unit Dose | |
|---|---|---|---|---|
| Group | Cohort | Study Intervention | (μg) | N |
| 1 | Phase IIb | RSV mRNA + LNP | 110 μg | 25 |
| | Sentinel | GL-HEPES-E3-E12- | | |
| | Cohort | DS-4-E10 | | |
| 2 | | Placebo | N/A | 25 |
| 1 | Phase IIb | RSV mRNA + LNP | 110 μg | 2595 |
| | Main Cohort | GL-HEPES-E3-E12- | | |
| | | DS-4-E10 | | |
| 2 | | Placebo | N/A | 2595 |
| 1 | Phase III | RSV mRNA + LNP | 110 μg | 4121 |
| | | GL-HEPES-E3-E12- | | |
| | | DS-4-E10 | | |
| 2 | | Placebo | N/A | 4121 |

For the Phase III Cohort, an additional 8242 participants (4121 participants per arm) are planned to be randomized. Approximately 2000 participants from the Phase IIb (1000 per arm) and 4000 participants (2000 per arm) from the Phase III Cohorts, will be included in a Reactogenicity Subset for the collection of solicited injection site and systemic reactions occurring up to 7 days after vaccination. Also, approximately 500 participants (250 per arm) in each of the Phase IIb and the Phase III Cohorts will be included in an Immunogenicity Subset to assess the durability of neutralizing antibody responses. Approximately 100 participants (50 per arm) in the Phase III Cohort will also be selected for inclusion in a CMI subset. Participants in the CMI subset will be enrolled from a limited number of selected sites that are chosen based on previous experience in collecting and processing blood samples for CMI assays.

Intervention Group. Intervention groups for Stage 2 will be eligible participants enrolled in a 1:1 ratio to receive a single intramuscular (IM) administration of the RSV mRNA vaccine candidate or placebo.

Anticipated Duration. The anticipated total duration of the study is approximately 6 months for participants in the Phase IIb Sentinel Cohort and approximate 12 months for participants in the Phase IIb Main Cohort/Phase III Cohort.

Composition. Participants in Stage 2 will receive by IM injection either a one dose (0.5 mL) liquid solution in a vial containing 110 μg of RSV pre-F mRNA with the selected LNP in a PBS 2.2× diluent or a one dose (0.5 mL) liquid solution in a vial containing 0.9% normal saline.

### B. Objectives

Primary Objectives. The primary objectives are to assess the safety of the RSV mRNA vaccine 110 μg dose with the selected LNP and to demonstrate with the same clinical efficacy of the mRNA RSV vaccine candidate for the prevention of RSV-LRTD during the first season occurring ≥14 days after vaccination.

Secondary Objectives. The secondary objectives are to demonstrate the clinical efficacy of the mRNA RSV vaccine candidate for the prevention of RSV-ARD (RSV-acute respiratory disease) and RSV-MAARD (RSV-medically attended acute respiratory disease) during the first season occurring ≥14 days after vaccination.

Table 13 below summarizes the primary objectives and the corresponding endpoints. Table 14 below summarizes the secondary objectives and the corresponding endpoints. Table 15 below summarizes the immunogenicity objectives and the corresponding endpoints. Table 16 below summarizes the safety objectives and the corresponding endpoints. Table 17 below summarizes the exploratory objectives and the corresponding endpoints.

TABLE 13
Primary Objectives and Corresponding Endpoints

| | |
|---|---|
| Objectives | Endpoints |

Primary

| | |
|---|---|
| Efficacy Objective | Efficacy Endpoints |
| (1) To demonstrate the clinical efficacy of | Occurrence of the first |
| the mRNA RSV vaccine candidate for the | episode of RSV-LRTD |
| prevention of RSV-LRTD during the first | (RSV A and/or RSV B) |
| season occurring ≥ 14 days after | during the first season ≥ |
| vaccination | 14 days after vaccination |

TABLE 14
Secondary Objectives and Corresponding Endpoints

| | |
|---|---|
| Objectives | Endpoints |

Key Secondary

| Efficacy Objective | Efficacy Endpoints |
|---|---|
| (1) To demonstrate the clinical efficacy of | Occurrence of the first episode of RSV-ARD |
| the mRNA RSV vaccine candidate for the | (RSV A and/or RSV B) during the first season ≥ |
| prevention of RSV-ARD during the first | 14 days after vaccination |
| season occurring ≥ 14 days after | |
| vaccination | |
| (2) To demonstrate the clinical efficacy of | Occurrence of the first episode of RSV- |
| the mRNA RSV vaccine candidate for the | MAARD (RSV A and/or RSV B) during the first |
| prevention of RSV-MAARD during the first | season ≥ 14 days after vaccination |
| season occurring ≥ 14 days after | |
| vaccination | |

Secondary

| | |
|---|---|
| (3) To describe the clinical efficacy | Occurrence of the first episode of RSV-LRTD |
| of the mRNA RSV vaccine | (RSV A) during the first season occurring ≥ 14 |
| candidate for the prevention of RSV- | days after vaccination |
| LRTD by RSV subtype and season | Occurrence of the first episode of RSV-LRTD |
| | (RSV B) during the first season occurring ≥ 14 |
| | days after vaccination |
| | Occurrence of the first episode of RSV-LRTD |
| | (RSV A) during the second season occurring ≥ |
| | 14 days after vaccination |
| | Occurrence of the first episode of RSV-LRTD |
| | (RSV B) during the second season occurring ≥ |
| | 14 days after vaccination |
| | Occurrence of the first episode of RSV-LRTD |
| | (RSV A and/or RSV B) during the second |
| | season occurring ≥ 14 days after vaccination |
| | Occurrence of the first episode of RSV-LRTD |
| | (RSV A) across 2 seasons occurring ≥ 14 days |
| | after vaccination |
| | Occurrence of the first episode of RSV-LRTD |
| | (RSV B) across 2 seasons occurring ≥ 14 days |
| | after vaccination |
| | Occurrence of the first episode of RSV-LRTD |
| | (RSV A and/or RSV B) across 2 seasons |
| | occurring ≥ 14 days after vaccination |
| (4) To describe the clinical efficacy | Occurrence of the first episode of RSV-LRTD |
| of the mRNA RSV vaccine | (RSV A and/or RSV B and by each one) during |
| candidate for the prevention of RSV- | the first, the second, and across 2 seasons by |
| LRTD during the first and second | age group |
| seasons, and across 2 seasons | Occurrence of the first episode of RSV-LRTD |
| occurring ≥ 14 days after | (RSV A and/or RSV B and by each one) during |
| vaccination by baseline stratification | the first, the second, and across 2 seasons by |
| factors | baseline comorbidities |
| | Occurrence of the first episode of RSV-LRTD |
| | (RSV A and/or RSV B and by each one) during |
| | the first, the second, and across 2 seasons by |
| | baseline frailty status^{a} |
| (5) To describe the clinical efficacy | Occurrence of the first episode of RSV-ARD |
| of the mRNA RSV vaccine | (RSV A and/or RSV B) across 2 seasons |
| candidate for the prevention of RSV- | Occurrence of the first episode of RSV-ARD |
| ARD occurring ≥ 14 days after | (RSV A and /or RSV B, and by each one) in |
| vaccination by season | the first season |
| | Occurrence of the first episode of RSV-ARD |
| | (RSV A and /or RSV B, and by each one) in |
| | the second season |
| (6) To describe the clinical efficacy | Occurrence of the first episode of RSV- |
| of the mRNA RSV vaccine | MAARD (RSV A and /or RSV B, and by each |
| candidate for the prevention of RSV- | one) across 2 seasons |
| medically attended ARD (MAARD) | Occurrence of the first episode of RSV- |
| occurring ≥ 14 days after | MAARD (RSV A and /or RSV B and by each |
| vaccination by season | one) in the first season |
| | Occurrence of the first episode of RSV- |
| | MAARD (RSV A and /or RSV B, and by each |
| | one) in the second season |
| (7) To describe the clinical efficacy | Occurrence of hospitalization due to RSV-ARD |
| of the mRNA RSV vaccine | and/or RSV-LRTD across 2 seasons |
| candidate for the prevention of | Occurrence of hospitalization due to RSV ARD |
| hospitalization due to RSV-ARD or | and/or RSV-LRTD in the first season |
| RSV-LRTD ≥ 14 days after | Occurrence of hospitalization due to RSV ARD |
| vaccination in the first and second | and/or RSV-LRTD in the second season |
| seasons, and across 2 seasons | |
| (8) To describe the duration of RSV- | Duration (number of days) of RSV-LRTD and |
| LRTD and RSV-ARD episodes | ARD episodes (RSV A and/or RSV B) |
| (RSV A and/or RSV B) | |
| (9) To describe the clinical efficacy | Occurrence of the first episode of severe RSV |
| of the mRNA RSV vaccine | LRTD (RSV A and/or RSV B and by each one) |
| candidate for the prevention of | during the first, the second, and across 2 |
| severe RSV-LRTD | seasons occurring ≥ 14 days after vaccination |

^{a}Baseline frailty status is assessed with the use of a gait speed test. A walking speed of < 0.4 m/second or an inability to perform the test indicates frail status, a walking speed of 0.4 to 0.99 m/second indicates pre-frail status, and a walking speed of 1 m/second or faster indicates fit status.

TABLE 15
Immunogenicity Objectives and Corresponding Endpoints

| Immunogenicity Objectives | Immunogenicity Endpoints |
|---|---|
| (1) To demonstrate the clinical efficacy of | Occurrence of the first episode of RSV-LRTD |
| the mRNA RSV vaccine candidate for the | (RSV A and/or RSV B) during the first season ≥ |
| prevention of RSV-LRTD during the first | 14 days after vaccination |
| season occurring ≥ 14 days after | |
| vaccination | |
| (2) To assess the durability of the immune | RSV A and RSV B serum nAb titers at 6 and |
| response at 6 and 12 months after | 12 months post-vaccination in the |
| vaccination in the Immunogenicity Subset | Immunogenicity Subset |
| | Serum anti-F IgG Ab titers at 6 and 12 months |
| | post-vaccination in the Immunogenicity Subset |

TABLE 16
Safety Objectives and Corresponding Endpoints

| Safety Objectives | Safety Endpoints |
|---|---|
| (1) To assess the safety of the mRNA RSV | For participants in the Safety Sentinel Cohort: |
| vaccine candidate throughout the study | Presence of out-of-range biological test results |
| | up to 7 days after vaccination |
| | For participants in the Safety Sentinel Cohort and |
| | Reactogenicity Subset: |
| | Presence of solicited injection site and |
| | systemic reactions (ie, pre-listed in the |
| | participant's diary and in the CRF) occurring up |
| | to 7 days after vaccination |
| | For all participants: |
| | Presence of unsolicited systemic AEs reported |
| | in the 30 minutes after vaccination |
| | Presence of unsolicited Aes reported up to |
| | 28 days after vaccination |
| | Presence of MAAEs within 6 months after |
| | vaccination |
| | Presence of SAEs and AESIs within 6 months |
| | after vaccination |
| | Presence of related SAEs, related AESIs, and |
| | fatal SAEs (regardless of causality) throughout |
| | the study |

TABLE 17
Exploratory Objectives and Corresponding Endpoints

| | |
|---|---|
| Objectives | Endpoints |

Exploratory

| | |
|---|---|
| (1) To assess the impact of the | Occurrence of ER visits due to RSV LRTD |
| mRNA RSV vaccine candidate in | and/or RSV-ARD across 2 seasons |
| the reduction of emergency room | Occurrence of ER visits due to RSV LRTD |
| (ER) visits due to RSV LRTD or | and/or RSV-ARD in the first season |
| RSV-ARD occurring ≥ 14 days after | Occurrence of ER visits due to RSV LRTD |
| vaccination by season | and/or RSV-ARD in the second season |
| (2) To assess the impact of the | Occurrence of outpatient clinic visits due to |
| mRNA RSV vaccine candidate in | RSV LRTD and/or RSV-ARD across 2 seasons |
| the reduction of outpatient clinic | Occurrence of outpatient clinic visits due to |
| visits due to RSV LRTD or RSV- | RSV LRTD and/or RSV-ARD in the first |
| ARD occurring ≥ 14 days after | season |
| vaccination by season | Occurrence of outpatient clinic visits due to |
| | RSV LRTD and/or RSv-ARD in the second |
| | season |
| (3) To assess the clinical efficacy of | Occurrence of RSV LRTD and RSV ARD ≥ 14 |
| the mRNA RSV vaccine candidate | days after vaccination concurrently with any |
| for the prevention of RSV-LRTD and | other respiratory pathogens in the first, the |
| RSV-ARD occurring ≥ 14 days after | second, and across 2 seasons |
| vaccination concurrently with other | Occurrence of RSV LRTD and RSV ARD ≥ 14 |
| selected pathogens by season | days after vaccination concurrently with hMPV |
| | in the first, the second, and across 2 seasons |
| | Occurrence of RSV-LRTD and RSV-ARD ≥ 14 |
| | days after vaccination concurrently with PIV3 |
| | in the first, the second, and across 2 seasons |
| (4) To describe the relationship | RSV A and RSV B serum nAb titers at D 01 |
| between post-dose neutralizing Ab | and D 29, in each study group |
| level and the subsequent | |
| occurrence of RSV-LRTD cases (All | |
| Participants) | |
| (5) To describe the relationship | RSV A and RSV B serum nAb titers at D 01 |
| between post-dose neutralizing Ab | and D 29, in each study group |
| level and the subsequent | |
| occurrence of RSV-ARD cases (All | |
| Participants) | RSV A and RSV B serum nAb titers at D 01 |
| (6) To describe the relationship | and D 29, in each study group |
| between post-dose neutralizing Ab | |
| level and the subsequent | |
| occurrence of Severe RSV LRTD | |
| cases by clinical symptoms (All | |
| Participants) | |
| (7) To describe the association | RSV A and RSV B serum nAb titers at D 01 |
| between post-dose neutralizing Ab | and D 29, in each study group |
| level and the subsequent | |
| occurrence of severe RSV LRTD | |
| cases by supportive therapy (All | |
| Participants) | |

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

1. A method of eliciting an immune response against respiratory syncytial virus (RSV) in a subject, the method comprising administering to the subject a prophylactically effective amount of an RSV vaccine comprising a messenger RNA (mRNA) comprising an open reading frame (ORF) encoding an RSV F protein antigen, wherein:
the RSV F protein antigen comprises an amino acid sequence with at least 98% identity to SEQ ID NO: 3 or consists of an amino acid sequence of SEQ ID NO: 3; and/or
the mRNA comprises a nucleic acid sequence with at least 98% identity to SEQ ID NO: 14 or consists of a nucleic acid sequence of SEQ ID NO: 14, optionally wherein the RSV F protein antigen is a pre-fusion protein.

2-3. (canceled)

4. The method of claim 1, wherein the RSV vaccine is administered intramuscularly, intranasally, intravenously, subcutaneously, or intradermally, optionally wherein the RSV vaccine is administered in a deltoid muscle of an upper arm of the subject.

5-6. (canceled)

7. The method of claim 1, wherein the subject is at least 60 years of age.

8. The method of claim 1, wherein the RSV vaccine does not comprise an adjuvant.

9. The method of claim 1, wherein the mRNA is formulated in a lipid nanoparticle (LNP).

10. The method of claim 9, wherein the LNP comprises at least one cationic lipid.

11-14. (canceled)

15. The method of claim 10, wherein the at least one cationic lipid is selected from the group consisting of OF-02, cKK-E10, GL-HEPES-E3-E10-DS-3-E18-1, GL-HEPES-E3-E12-DS-4-E10, and GL-HEPES-E3-E12-DS-3-E14, or IM-001.

16-18. (canceled)

19. The method of claim 1, wherein the subject is administered an initial dose of the RSV vaccine and one or more booster doses of the RSV vaccine, optionally wherein:
each of the one or more booster doses is administered to the subject at least 11 months after a previous dose;
each of the one or more booster doses is administered to the subject at least 12 months after a previous dose;
each of the one or more booster doses is administered to the subject about 10 months to about 14 months after a previous dose; or
each of the one or more booster doses is administered to the subject about 12 months after a previous dose.

20-28. (canceled)

29. The method of claim 1, wherein the RSV vaccine is administered at a dose of about 5 micrograms to about 120 micrograms.

30-32. (canceled)

33. The method of claim 29, wherein the RSV vaccine is administered at a dose of about 30 micrograms.

34. (canceled)

35. The method of claim 29, wherein the RSV vaccine is administered at a dose of about 75 micrograms.

36. (canceled)

37. The method of claim 29, wherein the RSV vaccine is administered at a dose of about 110 micrograms.

38. A method of preventing a respiratory syncytial virus (RSV) infection or reducing one or more symptoms of an RSV infection in a subject, the method comprising administering to the subject a prophylactically effective amount of an RSV vaccine comprising a messenger RNA (mRNA) comprising an open reading frame (ORF) encoding an RSV F protein antigen, wherein:
the RSV F protein antigen comprises an amino acid sequence with at least 98% identity to SEQ ID NO: 3 or consists of an amino acid sequence of SEQ ID NO: 3; and/or
the mRNA comprises a nucleic acid sequence with at least 98% identity to SEQ ID NO: 14 or consists of a nucleic acid sequence of SEQ ID NO: 14.

39-40. (canceled)

41. The method of claim 38, wherein the vaccine is administered intramuscularly, intranasally, intravenously, subcutaneously, or intradermally.

42-43. (canceled)

44. The method of claim 38, wherein the subject is at least 60 years of age.

45. (canceled)

46. The method of claim 38, wherein the mRNA is formulated in a lipid nanoparticle (LNP).

47. The method of claim 46, wherein the LNP comprises at least one cationic lipid.

48-51. (canceled)

52. The method of claim 47, wherein the at least one cationic lipid is selected from the group consisting of OF-02, cKK-E10, GL-HEPES-E3-E10-DS-3-E18-1, GL-HEPES-E3-E12-DS-4-E10, GL-HEPES-E3-E12-DS-3-E14, and IM-001.

53-65. (canceled)

66. The method of claim 38, wherein the RSV vaccine is administered at a dose of about 5 micrograms to about 120 micrograms.

67-69. (canceled)

70. The method of claim 66, wherein the RSV vaccine is administered at a dose of about 30 micrograms.

71. (canceled)

72. The method of claim 66, wherein the RSV vaccine is administered at a dose of about 75 micrograms.

73. (canceled)

74. The method of claim 66, wherein the RSV vaccine is administered at a dose of about 110 micrograms.

75. The method of claim 38, wherein the RSV vaccine is administered in a device suitable for skin injection.

76. The method of claim 38, wherein the one or more symptoms of an RSV infection are selected from the group consisting of acute respiratory disease (ARD), medically attended acute respiratory disease (MAARD), severe ARD, non-medically attended lower respiratory tract disease (LRTD), medically attended LRTD, congestion, runny nose, cough, fever, sore throat, headache, pneumonia, bronchiolitis, bronchopneumonia, and tracheobronchitis.

77. A method of eliciting an immune response against respiratory syncytial virus (RSV) in a subject and/or a method of preventing a respiratory syncytial virus (RSV) infection or reducing one or more symptoms of an RSV infection in a subject, the method comprising:
selecting a subject that is at least 60 years of age; and
administering to the subject a prophylactically effective amount of an RSV vaccine comprising a messenger RNA (mRNA) comprising an open reading frame (ORF) encoding an RSV F protein antigen, wherein:
the RSV F protein antigen comprises an amino acid sequence with at least 98% identity to SEQ ID NO: 3 or consists of an amino acid sequence of SEQ ID NO: 3; and/or
the mRNA comprises a nucleic acid sequence with at least 98% identity to SEQ ID NO: 14 or consists of a nucleic acid sequence of SEQ ID NO: 14.

78-94. (canceled)


SEQUENCE LISTING (21 sequences)

SEQ ID NO: 1 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 2 - protein, 544 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 3 - protein, 534 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVSKVLHLEGEVNKIKSALLSTNKAVVSLSNGVSVLTFKVLDLKNYIDKQLLPILNKQSCSISNPETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNELINQSLAFINQSDELLHNVNAGKSTTNIMITTIIIVIIVILLSLIAVGLLLYCKARSTPVTLSKDQLSGINNIAFSN

SEQ ID NO: 4 - RNA, 1725 nt, synthetic construct
  source 1..1725: mol_type = other RNA
  mRNA 1..1725: note = mRNA
atggaattgctgatcctcaaagcgaacgcaatcaccactatcctcactgcggtcaccttctgctttgcgagcggacagaacatcaccgaagaattctaccaatctacttgctccgccgtgtccaagggttacctgtccgccctgaggaccggatggtacacttccgtgattaccattgagttgtcgaatatcaagaagaacaagtgcaacggaaccgatgctaaggtcaagctgatcaagcaggagctggacaagtacaagaatgctgtgaccgagctgcagctgctgatgcagtccactcaagccaccaacaatcgcgcccggcgggaactcccaaggttcatgaactacaccttgaacaacgccaagaaaacgaacgtgaccctgtccaagaagcgcaagcgcagattccttggcttccttctgggcgtcggtagcgccatcgcctccggcgtggccgtcagcaaggtcctgcacctcgagggagaagtcaacaagattaagagcgccctgctgtccaccaacaaggccgtggtgtcgctatcaaacggcgtcagcgtactgaccagcaaagtgctggatctcaagaactacattgataagcaactcctccctatcgtgaataagcagagctgttcgatttccaacatcgagactgtgattgaattccagcagaagaacaaccggctgctggaaattaccagagaattcagcgtgaatgccggagtcactacccccgtgtccacctacatgctgacaaactccgagctgctgagcctgatcaacgatatgccgattaccaacgaccagaagaagctgatgtcgaacaacgtgcagatcgtgcgccagcagtcctactcaatcatgtcgatcatcaaggaagaggtcctggcctacgtggtgcagcttcctctgtacggcgtgattgacactccgtgttggaaactgcacactagtcccctgtgcactactaacaccaaggagggcagcaatatctgcctgactcggaccgatagaggctggtactgtgataacgccgggtccgtgtccttcttcccgcaagccgagacttgcaaagtgcagagcaaccgggtgttctgtgacactatgaactcactgaccttgccgagcgaagtcaacctttgcaacgtggacatctttaaccctaaatacgactgcaagatcatgacctccaagaccgacgtgtcgagctcagtgattacttcgctgggagccattgtgtcctgctacgggaaaaccaagtgcacggcctcaaacaagaaccggggtatcattaagaccttctccaacggctgcgactatgtgtccaacaagggggtggacactgtgtccgtgggaaacaccttgtattacgtgaacaagcaggagggaaagtccctctacgtgaagggcgaacccatcatcaatttctacgacccgctcgtgttcccctccgatgaattcgacgcatccatctcacaagtcaacgaaaagattaaccagtccctggctttcattcgcaagtccgacgaactgctccataacgtcaacgctggaaagtccaccaccaacatcatgatcaccacgatcattattgtgatcatcgtcatcctgctgtcactgatagcagtgggactgctcctctactgcaaagcgcggtcgaccccagtgacactctcgaaggaccagctgtccgggatcaacaacatcgcgttttcgaactga

SEQ ID NO: 5 - RNA, 1635 nt, synthetic construct
  source 1..1635: mol_type = other RNA
  mRNA 1..1635: note = mRNA
atggaactcctgatcctgaaggccaatgctatcactaccatcctgactgccgtcaccttctgcttcgcctccggacaaaatatcactgaagaattttaccaaagcacctgtagcgcggtgtccaagggatacctgagcgctctgaggaccggatggtacaccagcgtgattaccatcgagctgagtaacatcaagaagaacaagtgcaacgggaccgatgctaaggtcaagttgatcaaacaagagctcgacaagtacaagaacgccgtgactgagctgcagctgctgatgcagtcaactcaggccaccaacaaccgggccagacgggaactgccgagattcatgaactacaccctgaacaacgccaaaaagaccaacgtgaccctgtccaagaagagaaagcgccggttcctgggtttcctgcttggcgtgggatcagcaatcgcgtccggagtggcagtgtccaaggtcttgcacctcgagggcgaagtgaacaagatcaagtccgcgcttctgtcgaccaacaaggccgtcgtttccctgtcgaacggagtgtccgtgctcacgagcaaagtgctcgacctgaagaactacatcgacaaacagctgctgcccatcgtcaacaagcagagctgcagcatctcaaacattgaaaccgtgatcgagttccagcagaagaacaaccgcctgctcgagattaccagagagttttccgtgaacgccggcgtgaccaccccggtgtcgacctacatgctcacaaattcggaacttctctccctgattaatgacatgcccattactaacgatcagaaaaagctgatgtcgaacaatgtgcagattgtgcgccagcagtcctactccatcatgtccatcattaaggaagaggtcctggcctacgtggtgcagttgccgctgtacggtgtcatcgataccccctgctggaagctccatacttcgcccctgtgtactaccaacaccaaggaaggctccaacatctgcctgacccggacggatcgcggctggtactgtgacaatgccggatccgtgtcgttcttcccgcaagcggagacttgcaaagtgcagtccaaccgggtgttctgtgacactatgaactccctgactctgccgtccgaagtcaacctctgcaacgtggacattttcaatccaaaatacgactgcaagataatgacctccaagactgacgtgtcatcgtccgtgatcacatctctgggagccattgtctcctgctacggaaagactaagtgcaccgcgtcgaacaagaacaggggcattatcaagaccttcagcaacggttgcgactatgtgtccaacaagggcgtggataccgtgtccgtgggcaacaccttgtactacgtgaacaagcaggaggggaagtccctttatgtgaagggggagccaatcattaacttttacgaccccctggtgttccctagcgacgagttcgacgcctcaatctctcaagtcaacgaaaagatcaaccagagcctcgccttcatccgcaagtccgatgaactgctgtcagccattgggggttacatccctgaggcccctcgggacggacaggcatacgtccgcaaggacggcgaatgggtgctgcttagcaccttcctctaa

SEQ ID NO: 6 - RNA, 1605 nt, synthetic construct
  source 1..1605: mol_type = other RNA
  mRNA 1..1605: note = mRNA
atggaactgctgatcctcaaagccaacgcaatcaccaccattctcaccgctgtgaccttctgcttcgcatcggggcagaacatcactgaagagttttaccagagcacttgcagcgcggtgtcaaagggttacctttccgcactgcggaccggatggtacacttccgtgatcaccattgagctcagcaacatcaaggaaaacaagtgcaatggcaccgacgccaaggtcaagctgatcaaacaagaactggacaagtacaagaacgccgtgacagaattgcagctcctgatgggatccggaaacgtcggtctgggcggagccatcgcgagtggagtggctgtgtccaaggtcttgcacctcgagggagaagtgaacaagatcaagtccgcgctgctgtcaacgaacaaggccgtggtgtccctgtctaacggcgtcagcgtgctgacgttcaaggtcctggacctgaagaattacattgacaagcagctgctgcccatcctcaacaagcaatcctgctccatctccaaccccgaaaccgtgatcgagttccagcagaagaacaaccgcctgctggaaattactcgcgagttctctgtgaatgccggcgtgaccacccctgtgtccacctacatgctgaccaactccgagcttctctcccttatcaatgacatgcctatcacgaacgaccagaagaagctgatgtcgaacaacgtgcagattgtgcggcagcagtcatacagcatcatgtcgatcatcaaggaagaagtgctggcgtacgtggtgcaactcccgctgtacggcgtcatcgataccccgtgctggaagctgcacacctcgcctttgtgtaccaccaacaccaagaacggatccaacatctgcttaacccggactgatcggggttggtactgcgacaacgccgggaatgtttcgttcttcccacaagccgagacttgtaaagtgcagtcaaacagagtgttctgtgacaccatgaactcgagaaccctgcccagcgaagtgaacctgtgtaacgtcgacatctttaacccaaaatacgattgcaagattatgaccagcaaaaccgacgtgtcctcctccgtgataacaagcctgggggcgattgtgtcatgctacggaaagactaagtgcaccgcctcgaacaagaaccgcggcatcattaagactttctcgaatggttgcgactatgtgtccaacaagggcgtggatactgtgtcagtcgggaatactctttactacgtgaacaagcaggaggggaaaagcctctacgtgaagggagagcctattatcaacttttacgatccgctggtgttcccgtccgacgaattcgacgccagcatcagccaagtcaacgagctgattaaccagtccctcgccttcatcaaccaatccgacgagctcctgcataacgtgaacgccggaaagtccaccaccaacatcatgatcactactattatcatcgtgatcatcgtcatcctgctgagcctgattgctgtgggcctgttgctgtattgcaaagccaggtccaccccggtcaccctgtcgaaggatcagctgtccggaatcaacaacattgccttctccaactaa

SEQ ID NO: 7 - DNA, 1725 bp, synthetic construct
  source 1..1725: mol_type = other DNA
atggaattgctgatcctcaaagcgaacgcaatcaccactatcctcactgcggtcaccttctgctttgcgagcggacagaacatcaccgaagaattctaccaatctacttgctccgccgtgtccaagggttacctgtccgccctgaggaccggatggtacacttccgtgattaccattgagttgtcgaatatcaagaagaacaagtgcaacggaaccgatgctaaggtcaagctgatcaagcaggagctggacaagtacaagaatgctgtgaccgagctgcagctgctgatgcagtccactcaagccaccaacaatcgcgcccggcgggaactcccaaggttcatgaactacaccttgaacaacgccaagaaaacgaacgtgaccctgtccaagaagcgcaagcgcagattccttggcttccttctgggcgtcggtagcgccatcgcctccggcgtggccgtcagcaaggtcctgcacctcgagggagaagtcaacaagattaagagcgccctgctgtccaccaacaaggccgtggtgtcgctatcaaacggcgtcagcgtactgaccagcaaagtgctggatctcaagaactacattgataagcaactcctccctatcgtgaataagcagagctgttcgatttccaacatcgagactgtgattgaattccagcagaagaacaaccggctgctggaaattaccagagaattcagcgtgaatgccggagtcactacccccgtgtccacctacatgctgacaaactccgagctgctgagcctgatcaacgatatgccgattaccaacgaccagaagaagctgatgtcgaacaacgtgcagatcgtgcgccagcagtcctactcaatcatgtcgatcatcaaggaagaggtcctggcctacgtggtgcagcttcctctgtacggcgtgattgacactccgtgttggaaactgcacactagtcccctgtgcactactaacaccaaggagggcagcaatatctgcctgactcggaccgatagaggctggtactgtgataacgccgggtccgtgtccttcttcccgcaagccgagacttgcaaagtgcagagcaaccgggtgttctgtgacactatgaactcactgaccttgccgagcgaagtcaacctttgcaacgtggacatctttaaccctaaatacgactgcaagatcatgacctccaagaccgacgtgtcgagctcagtgattacttcgctgggagccattgtgtcctgctacgggaaaaccaagtgcacggcctcaaacaagaaccggggtatcattaagaccttctccaacggctgcgactatgtgtccaacaagggggtggacactgtgtccgtgggaaacaccttgtattacgtgaacaagcaggagggaaagtccctctacgtgaagggcgaacccatcatcaatttctacgacccgctcgtgttcccctccgatgaattcgacgcatccatctcacaagtcaacgaaaagattaaccagtccctggctttcattcgcaagtccgacgaactgctccataacgtcaacgctggaaagtccaccaccaacatcatgatcaccacgatcattattgtgatcatcgtcatcctgctgtcactgatagcagtgggactgctcctctactgcaaagcgcggtcgaccccagtgacactctcgaaggaccagctgtccgggatcaacaacatcgcgttttcgaactga

SEQ ID NO: 8 - DNA, 1635 bp, synthetic construct
  source 1..1635: mol_type = other DNA
atggaactcctgatcctgaaggccaatgctatcactaccatcctgactgccgtcaccttctgcttcgcctccggacaaaatatcactgaagaattttaccaaagcacctgtagcgcggtgtccaagggatacctgagcgctctgaggaccggatggtacaccagcgtgattaccatcgagctgagtaacatcaagaagaacaagtgcaacgggaccgatgctaaggtcaagttgatcaaacaagagctcgacaagtacaagaacgccgtgactgagctgcagctgctgatgcagtcaactcaggccaccaacaaccgggccagacgggaactgccgagattcatgaactacaccctgaacaacgccaaaaagaccaacgtgaccctgtccaagaagagaaagcgccggttcctgggtttcctgcttggcgtgggatcagcaatcgcgtccggagtggcagtgtccaaggtcttgcacctcgagggcgaagtgaacaagatcaagtccgcgcttctgtcgaccaacaaggccgtcgtttccctgtcgaacggagtgtccgtgctcacgagcaaagtgctcgacctgaagaactacatcgacaaacagctgctgcccatcgtcaacaagcagagctgcagcatctcaaacattgaaaccgtgatcgagttccagcagaagaacaaccgcctgctcgagattaccagagagttttccgtgaacgccggcgtgaccaccccggtgtcgacctacatgctcacaaattcggaacttctctccctgattaatgacatgcccattactaacgatcagaaaaagctgatgtcgaacaatgtgcagattgtgcgccagcagtcctactccatcatgtccatcattaaggaagaggtcctggcctacgtggtgcagttgccgctgtacggtgtcatcgataccccctgctggaagctccatacttcgcccctgtgtactaccaacaccaaggaaggctccaacatctgcctgacccggacggatcgcggctggtactgtgacaatgccggatccgtgtcgttcttcccgcaagcggagacttgcaaagtgcagtccaaccgggtgttctgtgacactatgaactccctgactctgccgtccgaagtcaacctctgcaacgtggacattttcaatccaaaatacgactgcaagataatgacctccaagactgacgtgtcatcgtccgtgatcacatctctgggagccattgtctcctgctacggaaagactaagtgcaccgcgtcgaacaagaacaggggcattatcaagaccttcagcaacggttgcgactatgtgtccaacaagggcgtggataccgtgtccgtgggcaacaccttgtactacgtgaacaagcaggaggggaagtccctttatgtgaagggggagccaatcattaacttttacgaccccctggtgttccctagcgacgagttcgacgcctcaatctctcaagtcaacgaaaagatcaaccagagcctcgccttcatccgcaagtccgatgaactgctgtcagccattgggggttacatccctgaggcccctcgggacggacaggcatacgtccgcaaggacggcgaatgggtgctgcttagcaccttcctctaa

SEQ ID NO: 9 - DNA, 1605 bp, synthetic construct
  source 1..1605: mol_type = other DNA
atggaactgctgatcctcaaagccaacgcaatcaccaccattctcaccgctgtgaccttctgcttcgcatcggggcagaacatcactgaagagttttaccagagcacttgcagcgcggtgtcaaagggttacctttccgcactgcggaccggatggtacacttccgtgatcaccattgagctcagcaacatcaaggaaaacaagtgcaatggcaccgacgccaaggtcaagctgatcaaacaagaactggacaagtacaagaacgccgtgacagaattgcagctcctgatgggatccggaaacgtcggtctgggcggagccatcgcgagtggagtggctgtgtccaaggtcttgcacctcgagggagaagtgaacaagatcaagtccgcgctgctgtcaacgaacaaggccgtggtgtccctgtctaacggcgtcagcgtgctgacgttcaaggtcctggacctgaagaattacattgacaagcagctgctgcccatcctcaacaagcaatcctgctccatctccaaccccgaaaccgtgatcgagttccagcagaagaacaaccgcctgctggaaattactcgcgagttctctgtgaatgccggcgtgaccacccctgtgtccacctacatgctgaccaactccgagcttctctcccttatcaatgacatgcctatcacgaacgaccagaagaagctgatgtcgaacaacgtgcagattgtgcggcagcagtcatacagcatcatgtcgatcatcaaggaagaagtgctggcgtacgtggtgcaactcccgctgtacggcgtcatcgataccccgtgctggaagctgcacacctcgcctttgtgtaccaccaacaccaagaacggatccaacatctgcttaacccggactgatcggggttggtactgcgacaacgccgggaatgtttcgttcttcccacaagccgagacttgtaaagtgcagtcaaacagagtgttctgtgacaccatgaactcgagaaccctgcccagcgaagtgaacctgtgtaacgtcgacatctttaacccaaaatacgattgcaagattatgaccagcaaaaccgacgtgtcctcctccgtgataacaagcctgggggcgattgtgtcatgctacggaaagactaagtgcaccgcctcgaacaagaaccgcggcatcattaagactttctcgaatggttgcgactatgtgtccaacaagggcgtggatactgtgtcagtcgggaatactctttactacgtgaacaagcaggaggggaaaagcctctacgtgaagggagagcctattatcaacttttacgatccgctggtgttcccgtccgacgaattcgacgccagcatcagccaagtcaacgagctgattaaccagtccctcgccttcatcaaccaatccgacgagctcctgcataacgtgaacgccggaaagtccaccaccaacatcatgatcactactattatcatcgtgatcatcgtcatcctgctgagcctgattgctgtgggcctgttgctgtattgcaaagccaggtccaccccggtcaccctgtcgaaggatcagctgtccggaatcaacaacattgccttctccaactaa

SEQ ID NO: 10 - RNA, 140 nt, synthetic construct
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 11 - RNA, 100 nt, synthetic construct
  source 1..100: mol_type = other RNA
cgggtggcatccctgtgacccctccccagtgcctctcctggccctggaagttgccactccagtgcccaccagccttgtcctaataaaattaagttgcatc

SEQ ID NO: 12 - RNA, 1965 nt, synthetic construct
  source 1..1965: mol_type = other RNA
  mRNA 1..1965: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggaattgctgatcctcaaagcgaacgcaatcaccactatcctcactgcggtcaccttctgctttgcgagcggacagaacatcaccgaagaattctaccaatctacttgctccgccgtgtccaagggttacctgtccgccctgaggaccggatggtacacttccgtgattaccattgagttgtcgaatatcaagaagaacaagtgcaacggaaccgatgctaaggtcaagctgatcaagcaggagctggacaagtacaagaatgctgtgaccgagctgcagctgctgatgcagtccactcaagccaccaacaatcgcgcccggcgggaactcccaaggttcatgaactacaccttgaacaacgccaagaaaacgaacgtgaccctgtccaagaagcgcaagcgcagattccttggcttccttctgggcgtcggtagcgccatcgcctccggcgtggccgtcagcaaggtcctgcacctcgagggagaagtcaacaagattaagagcgccctgctgtccaccaacaaggccgtggtgtcgctatcaaacggcgtcagcgtactgaccagcaaagtgctggatctcaagaactacattgataagcaactcctccctatcgtgaataagcagagctgttcgatttccaacatcgagactgtgattgaattccagcagaagaacaaccggctgctggaaattaccagagaattcagcgtgaatgccggagtcactacccccgtgtccacctacatgctgacaaactccgagctgctgagcctgatcaacgatatgccgattaccaacgaccagaagaagctgatgtcgaacaacgtgcagatcgtgcgccagcagtcctactcaatcatgtcgatcatcaaggaagaggtcctggcctacgtggtgcagcttcctctgtacggcgtgattgacactccgtgttggaaactgcacactagtcccctgtgcactactaacaccaaggagggcagcaatatctgcctgactcggaccgatagaggctggtactgtgataacgccgggtccgtgtccttcttcccgcaagccgagacttgcaaagtgcagagcaaccgggtgttctgtgacactatgaactcactgaccttgccgagcgaagtcaacctttgcaacgtggacatctttaaccctaaatacgactgcaagatcatgacctccaagaccgacgtgtcgagctcagtgattacttcgctgggagccattgtgtcctgctacgggaaaaccaagtgcacggcctcaaacaagaaccggggtatcattaagaccttctccaacggctgcgactatgtgtccaacaagggggtggacactgtgtccgtgggaaacaccttgtattacgtgaacaagcaggagggaaagtccctctacgtgaagggcgaacccatcatcaatttctacgacccgctcgtgttcccctccgatgaattcgacgcatccatctcacaagtcaacgaaaagattaaccagtccctggctttcattcgcaagtccgacgaactgctccataacgtcaacgctggaaagtccaccaccaacatcatgatcaccacgatcattattgtgatcatcgtcatcctgctgtcactgatagcagtgggactgctcctctactgcaaagcgcggtcgaccccagtgacactctcgaaggaccagctgtccgggatcaacaacatcgcgttttcgaactgacgggtggcatccctgtgacccctccccagtgcctctcctggccctggaagttgccactccagtgcccaccagccttgtcctaataaaattaagttgcatc

SEQ ID NO: 13 - RNA, 1875 nt, synthetic construct
  source 1..1875: mol_type = other RNA
  mRNA 1..1875: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggaactcctgatcctgaaggccaatgctatcactaccatcctgactgccgtcaccttctgcttcgcctccggacaaaatatcactgaagaattttaccaaagcacctgtagcgcggtgtccaagggatacctgagcgctctgaggaccggatggtacaccagcgtgattaccatcgagctgagtaacatcaagaagaacaagtgcaacgggaccgatgctaaggtcaagttgatcaaacaagagctcgacaagtacaagaacgccgtgactgagctgcagctgctgatgcagtcaactcaggccaccaacaaccgggccagacgggaactgccgagattcatgaactacaccctgaacaacgccaaaaagaccaacgtgaccctgtccaagaagagaaagcgccggttcctgggtttcctgcttggcgtgggatcagcaatcgcgtccggagtggcagtgtccaaggtcttgcacctcgagggcgaagtgaacaagatcaagtccgcgcttctgtcgaccaacaaggccgtcgtttccctgtcgaacggagtgtccgtgctcacgagcaaagtgctcgacctgaagaactacatcgacaaacagctgctgcccatcgtcaacaagcagagctgcagcatctcaaacattgaaaccgtgatcgagttccagcagaagaacaaccgcctgctcgagattaccagagagttttccgtgaacgccggcgtgaccaccccggtgtcgacctacatgctcacaaattcggaacttctctccctgattaatgacatgcccattactaacgatcagaaaaagctgatgtcgaacaatgtgcagattgtgcgccagcagtcctactccatcatgtccatcattaaggaagaggtcctggcctacgtggtgcagttgccgctgtacggtgtcatcgataccccctgctggaagctccatacttcgcccctgtgtactaccaacaccaaggaaggctccaacatctgcctgacccggacggatcgcggctggtactgtgacaatgccggatccgtgtcgttcttcccgcaagcggagacttgcaaagtgcagtccaaccgggtgttctgtgacactatgaactccctgactctgccgtccgaagtcaacctctgcaacgtggacattttcaatccaaaatacgactgcaagataatgacctccaagactgacgtgtcatcgtccgtgatcacatctctgggagccattgtctcctgctacggaaagactaagtgcaccgcgtcgaacaagaacaggggcattatcaagaccttcagcaacggttgcgactatgtgtccaacaagggcgtggataccgtgtccgtgggcaacaccttgtactacgtgaacaagcaggaggggaagtccctttatgtgaagggggagccaatcattaacttttacgaccccctggtgttccctagcgacgagttcgacgcctcaatctctcaagtcaacgaaaagatcaaccagagcctcgccttcatccgcaagtccgatgaactgctgtcagccattgggggttacatccctgaggcccctcgggacggacaggcatacgtccgcaaggacggcgaatgggtgctgcttagcaccttcctctaacgggtggcatccctgtgacccctccccagtgcctctcctggccctggaagttgccactccagtgcccaccagccttgtcctaataaaattaagttgcatc

SEQ ID NO: 14 - RNA, 1845 nt, synthetic construct
  source 1..1845: mol_type = other RNA
  mRNA 1..1845: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggaactgctgatcctcaaagccaacgcaatcaccaccattctcaccgctgtgaccttctgcttcgcatcggggcagaacatcactgaagagttttaccagagcacttgcagcgcggtgtcaaagggttacctttccgcactgcggaccggatggtacacttccgtgatcaccattgagctcagcaacatcaaggaaaacaagtgcaatggcaccgacgccaaggtcaagctgatcaaacaagaactggacaagtacaagaacgccgtgacagaattgcagctcctgatgggatccggaaacgtcggtctgggcggagccatcgcgagtggagtggctgtgtccaaggtcttgcacctcgagggagaagtgaacaagatcaagtccgcgctgctgtcaacgaacaaggccgtggtgtccctgtctaacggcgtcagcgtgctgacgttcaaggtcctggacctgaagaattacattgacaagcagctgctgcccatcctcaacaagcaatcctgctccatctccaaccccgaaaccgtgatcgagttccagcagaagaacaaccgcctgctggaaattactcgcgagttctctgtgaatgccggcgtgaccacccctgtgtccacctacatgctgaccaactccgagcttctctcccttatcaatgacatgcctatcacgaacgaccagaagaagctgatgtcgaacaacgtgcagattgtgcggcagcagtcatacagcatcatgtcgatcatcaaggaagaagtgctggcgtacgtggtgcaactcccgctgtacggcgtcatcgataccccgtgctggaagctgcacacctcgcctttgtgtaccaccaacaccaagaacggatccaacatctgcttaacccggactgatcggggttggtactgcgacaacgccgggaatgtttcgttcttcccacaagccgagacttgtaaagtgcagtcaaacagagtgttctgtgacaccatgaactcgagaaccctgcccagcgaagtgaacctgtgtaacgtcgacatctttaacccaaaatacgattgcaagattatgaccagcaaaaccgacgtgtcctcctccgtgataacaagcctgggggcgattgtgtcatgctacggaaagactaagtgcaccgcctcgaacaagaaccgcggcatcattaagactttctcgaatggttgcgactatgtgtccaacaagggcgtggatactgtgtcagtcgggaatactctttactacgtgaacaagcaggaggggaaaagcctctacgtgaagggagagcctattatcaacttttacgatccgctggtgttcccgtccgacgaattcgacgccagcatcagccaagtcaacgagctgattaaccagtccctcgccttcatcaaccaatccgacgagctcctgcataacgtgaacgccggaaagtccaccaccaacatcatgatcactactattatcatcgtgatcatcgtcatcctgctgagcctgattgctgtgggcctgttgctgtattgcaaagccaggtccaccccggtcaccctgtcgaaggatcagctgtccggaatcaacaacattgccttctccaactaacgggtggcatccctgtgacccctccccagtgcctctcctggccctggaagttgccactccagtgcccaccagccttgtcctaataaaattaagttgcatc

SEQ ID NO: 15 - protein, 7 aa, synthetic construct
GSGNVGL

SEQ ID NO: 16 - protein, 9 aa, synthetic construct
GSGNVGLGG

SEQ ID NO: 17 - protein, 50 aa, synthetic construct
IMITTIIIVIIVILLSLIAVGLLLYCKARSTPVTLSKDQLSGINNIAFSN

SEQ ID NO: 18 - RNA, 11 nt, synthetic construct
  source 1..11: mol_type = other RNA
gggatcctacc

SEQ ID NO: 19 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 20 - RNA, 10 nt, synthetic construct
  source 1..10: mol_type = other RNA
  repeat_region 1: note = The A Residue can be repeated upto five hundred times.
aaaaaaaaaa

SEQ ID NO: 21 - RNA, 10 nt, synthetic construct
  source 1..10: mol_type = other RNA
  repeat_region 1: note = The C Residue can be repeated upto two hundred times.
cccccccccc